SEQUENCE LISTING (95 sequences)

SEQ ID NO: 1 - protein, 12 aa, Synthetic construct
PDRKAAVSHWQQ

SEQ ID NO: 2 - protein, 12 aa, synthetic construct
PDRKAAVAHWQQ

SEQ ID NO: 3 - protein, 12 aa, synthetic construct
PDRKAAVTHWQQ

SEQ ID NO: 4 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSS

SEQ ID NO: 5 - protein, 14 aa, synthetic construct
ARGCKRGRYEYDFW

SEQ ID NO: 6 - protein, 14 aa, synthetic construct
ARGCKRGAYEYDFW

SEQ ID NO: 7 - protein, 14 aa, synthetic construct
ARGCKRGEYEYDFW

SEQ ID NO: 8 - protein, 14 aa, synthetic construct
ARGCKRGSYEYDFW

SEQ ID NO: 9 - protein, 7 aa, synthetic construct
QGQSGQG

SEQ ID NO: 10 - protein, 7 aa, synthetic construct
QVQLVES

SEQ ID NO: 11 - protein, 14 aa, synthetic construct
GGGGSPLGLAGGGS

SEQ ID NO: 12 - protein, 14 aa, synthetic construct
GGGGSLALGPGGGS

SEQ ID NO: 13 - protein, 6 aa, synthetic construct
PLGLAG

SEQ ID NO: 14 - protein, 8 aa, synthetic construct
LSGRSDNH

SEQ ID NO: 15 - protein, 6 aa, synthetic construct
ISSGLL

SEQ ID NO: 16 - protein, 13 aa, synthetic construct
ISSGLLSGRSDNH

SEQ ID NO: 17 - protein, 22 aa, synthetic construct
ISSGLLSSGGSGGSLSGRSDNH

SEQ ID NO: 18 - protein, 22 aa, synthetic construct
LSGRSDNHGGSGGSISSGLLSS

SEQ ID NO: 19 - protein, 12 aa, synthetic construct
GGSPLGLAGGGS

SEQ ID NO: 20 - protein, 5 aa, synthetic construct
GGGGS

SEQ ID NO: 21 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 449 aa, synthetic construct
EVQLVESGGGLVQPGGSLRLSCAASGFNIKDTYIHWVRQACGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 446 aa, synthetic construct
QIQLVQSGPEVKKPGESVKISCKASGYTFTKYGMNWVKQAPGQGLKWMGWINTYTEEPTYGDDFKGRFTFTLDTSTSTAYLEISSLRSEDTATYFCARFGSAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 446 aa, synthetic construct
QIQLVQSGPEVKKPGESVKISCKASGYTFTKYGMNWVKQACGQGLKWMGWINTYTEEPTYGDDFKGRFTFTLDTSTSTAYLEISSLRSEDTATYFCARFGSAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 219 aa, synthetic construct
DIVMTQSALSNPVTLGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 457 aa, synthetic construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 457 aa, synthetic construct
EVQLLESGGGVVQPGRSLRLSCAASGFTFSSYGMHWVRQACGKGLEWVAVISYDGSNKYYADSVKGRFTISRDNSKNTLYLQMNSLRAEDTAVYYCAKDMGWGSGWRPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 214 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 451 aa, synthetic construct
QVQLQQSGSELKKPGASVKVSCKASGYTFTNYGMNWVKQACGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 447 aa, synthetic construct
QVQLVESGGGVVQPGRSLRLSCKASGFNIKDYYMHWVRQAPGKGLEWIGLIDPENGNTIYDPKFQGRFTISADNSKNTLFLQMDSLRPEDTAVYYCARDNSYYFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 447 aa, synthetic construct
QVQLVESGGGVVQPGRSLRLSCKASGFNIKDYYMHWVRQACGKGLEWIGLIDPENGNTIYDPKFQGRFTISADNSKNTLFLQMDSLRPEDTAVYYCARDNSYYFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 214 aa, synthetic construct
DIQMTQSPSSLSASVGDRVTITCKASQDIRKYLNWYQQKPGKAPKLLIYYATSLADGVPSRFSGSGSGTDYTFTISSLQPEDIATYYCLQHGESPYTFGQGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 448 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 448 aa, synthetic construct
EVQLLESGGGLVQPGGSLRLSCAASGFTFSNYAMSWVRQACGKGLEWVSSISGSGDYTYYTDSVKGRFTISRDNSKNTLYLQMNSLRAEDTAVYYCARSPWGYYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 448 aa, synthetic construct
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 216 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 451 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 42 - protein, 214 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 21 aa, synthetic construct
MACPGFLWALVISTCLEFSMA

SEQ ID NO: 44 - protein, 12 aa, synthetic construct
PDRVRAVSHWSS

SEQ ID NO: 45 - protein, 4 aa, synthetic construct
EPEA

SEQ ID NO: 46 - protein, 13 aa, synthetic construct
SRLEEELRRRLTE

SEQ ID NO: 47 - protein, 122 aa, synthetic construct
EVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 48 - protein, 12 aa, synthetic construct
PVRSAALSQWSS

SEQ ID NO: 49 - protein, 12 aa, synthetic construct
PDRVRAVSHWSS

SEQ ID NO: 50 - protein, 12 aa, synthetic construct
ADRVRAVSHWSS

SEQ ID NO: 51 - protein, 12 aa, synthetic construct
  VARIANT 1: note = X1 can be P or A
  VARIANT 2: note = D2 can be replaced by a conservative substitution of D
  VARIANT 4: note = X4 can be K, or a conservative substitution of K, or S
  VARIANT 5: note = X5 can be A or R, or a conservative substitution of A or R
  VARIANT 7: note = V7 can be replaced by a conservative substitution of V
  VARIANT 8: note = X8 can be S, A, V or T
  VARIANT 9: note = H9 can be replaced by a conservative substitution of H
  VARIANT 11: note = Q11 can be replaced by a conservative substitution of Q
  VARIANT 12: note = Q12 can be replaced by a conservative substitution of Q
XDRXXAVXHWQQ

SEQ ID NO: 52 - protein, 6 aa, synthetic construct
IPVSLR

SEQ ID NO: 53 - protein, 8 aa, synthetic construct
RQARVVNG

SEQ ID NO: 54 - protein, 5 aa, synthetic construct
PMAKK

SEQ ID NO: 55 - protein, 10 aa, synthetic construct
GGLSGRSDNH

SEQ ID NO: 56 - protein, 11 aa, synthetic construct
SGGLSGRSDNH

SEQ ID NO: 57 - protein, 12 aa, synthetic construct
GSGGLSGRSDNH

SEQ ID NO: 58 - protein, 13 aa, synthetic construct
GGLSGRSDNHGGS

SEQ ID NO: 59 - protein, 14 aa, synthetic construct
SGGLSGRSDNHGGS

SEQ ID NO: 60 - protein, 15 aa, synthetic construct
GSGGLSGRSDNHGGS

SEQ ID NO: 61 - protein, 16 aa, synthetic construct
GGSGGLSGRSDNHGGS

SEQ ID NO: 62 - protein, 17 aa, synthetic construct
GGGSGGLSGRSDNHGGS

SEQ ID NO: 63 - protein, 18 aa, synthetic construct
GGGGSGGLSGRSDNHGGS

SEQ ID NO: 64 - protein, 21 aa, synthetic construct
GGGGSGGSGGLSGRSDNHGGS

SEQ ID NO: 65 - protein, 24 aa, synthetic construct
GGGSSGGSGGSGGLSGRSDNHGGS

SEQ ID NO: 66 - protein, 16 aa, synthetic construct
GGGSGGSPLGLAGGGS

SEQ ID NO: 67 - protein, 15 aa, synthetic construct
GGSGGSPLGLAGGGS

SEQ ID NO: 68 - protein, 13 aa, synthetic construct
GGGSPLGLAGGGS

SEQ ID NO: 69 - protein, 11 aa, synthetic construct
GGPLGLAGGGS

SEQ ID NO: 70 - protein, 5 aa, synthetic construct
PLGVR

SEQ ID NO: 71 - protein, 330 aa, synthetic construct [sequence omitted]

SEQ ID NO: 72 - protein, 107 aa, synthetic construct
DIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 73 - protein, 451 aa, synthetic construct
QVQLQQPGAELVKPGASVKMSCKASGYTFTSYNMHWVKQTCGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 8 aa, synthetic construct
IPVSLRSG

SEQ ID NO: 76 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGAYEYDFWGQGTQVTVSS

SEQ ID NO: 77 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGEYEYDFWGQGTQVTVSS

SEQ ID NO: 78 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGSYEYDFWGQGTQVTVSS

SEQ ID NO: 79 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLRAEDTAIYYCAEARGCKRGRYEYDFWGQGTQVTVSS

SEQ ID NO: 80 - protein, 122 aa, synthetic construct
QVQLVESGGGLVQPGGSLRLSCTASGFTLDHYDIGWFRQAPGKEREGVSCINNSDDDTYYADSVKGRFTIFMNNAKDTVYLQMNSLRAEDTAIYYCAEARGCKRGRYEYDFWGQGTQVTVSS

SEQ ID NO: 81 - protein, 122 aa, synthetic construct
QVQLVESGGGSVQPGGSLRLSCTASGFTLDHYDIGWFRQAPGKEREGVSCINNSDDDTYYADSVKGRFTIFMNNAKDTVYLQMNSLRAEDTAIYYCAEARGCKRGRYEYDFWGQGTQVTVSS

SEQ ID NO: 82 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLRAEDTAIYYCAEARGCKRGAYEYDFWGQGTQVTVSS

SEQ ID NO: 83 - protein, 122 aa, synthetic construct
QVQLVESGGGLVQPGGSLRLSCTASGFTLDHYDIGWFRQAPGKEREGVSCINNSDDDTYYADSVKGRFTIFMNNAKDTVYLQMNSLRAEDTAIYYCAEARGCKRGAYEYDFWGQGTQVTVSS

SEQ ID NO: 84 - protein, 122 aa, synthetic construct
QVQLVESGGGSVQPGGSLRLSCTASGFTLDHYDIGWFRQAPGKEREGVSCINNSDDDTYYADSVKGRFTIFMNNAKDTVYLQMNSLRAEDTAIYYCAEARGCKRGAYEYDFWGQGTQVTVSS

SEQ ID NO: 85 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLRAEDTAIYYCAEARGCKRGEYEYDFWGQGTQVTVSS

SEQ ID NO: 86 - protein, 122 aa, synthetic construct
QVQLVESGGGLVQPGGSLRLSCTASGFTLDHYDIGWFRQAPGKEREGVSCINNSDDDTYYADSVKGRFTIFMNNAKDTVYLQMNSLRAEDTAIYYCAEARGCKRGEYEYDFWGQGTQVTVSS

SEQ ID NO: 87 - protein, 122 aa, synthetic construct
QVQLVESGGGSVQPGGSLRLSCTASGFTLDHYDIGWFRQAPGKEREGVSCINNSDDDTYYADSVKGRFTIFMNNAKDTVYLQMNSLRAEDTAIYYCAEARGCKRGEYEYDFWGQGTQVTVSS

SEQ ID NO: 88 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLRAEDTAIYYCAEARGCKRGSYEYDFWGQGTQVTVSS

SEQ ID NO: 89 - protein, 122 aa, synthetic construct
QVQLVESGGGLVQPGGSLRLSCTASGFTLDHYDIGWFRQAPGKEREGVSCINNSDDDTYYADSVKGRFTIFMNNAKDTVYLQMNSLRAEDTAIYYCAEARGCKRGSYEYDFWGQGTQVTVSS

SEQ ID NO: 90 - protein, 122 aa, synthetic construct
QVQLVESGGGSVQPGGSLRLSCTASGFTLDHYDIGWFRQAPGKEREGVSCINNSDDDTYYADSVKGRFTIFMNNAKDTVYLQMNSLRAEDTAIYYCAEARGCKRGSYEYDFWGQGTQVTVSS

SEQ ID NO: 91 - protein, 122 aa, synthetic construct
EVQLQESGGGLVQPGGSLRLSCTASGVTISALNAMAMGWYRQAPGEERVMVAAVSSRGNAMYRESVQGRFTVTRDFTNKMVSLQMDNLKPEDTAVYYCHVLEDRVDSFHDYWGQGTQVTVSS

SEQ ID NO: 92 - protein, 15 aa, synthetic construct
PSRLEEELRRRLTEP

SEQ ID NO: 93 - protein, 19 aa, synthetic construct
GGGGSGGGGSPLGLAGGGS

SEQ ID NO: 94 - protein, 9 aa, synthetic construct
PLGLAGGGS

SEQ ID NO: 95 - protein, 12 aa, synthetic construct
  VARIANT 3: note = X3 can be R or K
  VARIANT 1: note = X1 can be P or A
  VARIANT 12: note = Q12 can be replaced by a conservative substitution of Q
  VARIANT 9: note = H9 can be replaced by a conservative substitution of H
  VARIANT 11: note = Q11 can be replaced by a conservative substitution of Q
  VARIANT 2: note = D2 can be replaced by a conservative substitution of D
  VARIANT 5: note = X5 can be A or R, or a conservative substitution of A or R
  VARIANT 7: note = V7 can be replaced by a conservative substitution of V
  VARIANT 4: note = X4 can be K, or a conservative substitution of K, or S
  VARIANT 8: note = X8 can be S, A, V or T
XDXXXAVXHWQQ

(19) United States
(12) Patent Application Publication
(10) Pub. No.: US 2025/0136717 A1
(43) Pub. Date: May 1, 2025

(54) NOVEL MASKED ANTIBODIES

(71) Applicant: BYONDIS B.V., Nijmegen (NL)

(72) Inventors: Rudy Gerardus Elisabeth COUMANS, Nijmegen (NL); Gerard ROUWENDAL, Nijmegen (NL)

(21) Appl. No.: 18/865,949

(22) PCT Filed: May 15, 2023
(86) PCT No.: PCT/EP2023/062922

§ 371 Date: Nov. 14, 2024

(30) Foreign Application Priority Data
May 16, 2022 (EP) 22173568.1

(51) Int. Cl. C07K 16/36; A61K 47/68; A61P 35/00

(52) U.S. Cl. CPC C07K 16/36; A61K 47/68031; A61K 47/6851; A61P 35/00; C07K 2317/732; C07K 2317/92; C07K 2319/50

(57) ABSTRACT

The present invention provides a masked antibody or antigen binding fragment thereof, comprising at least one light chain (LC) variable domain and heavy chain (HC) variable domain pair provided with a mask, said mask comprising two peptidic masking moieties specifically binding to each other, linked to the N-termini of the light and heavy chain variable domains, respectively, wherein at least one masking moiety is linked via a cleavable linker, characterized in that one of the two masking moieties comprises an antigenic peptide, while the other comprises a sdAb specifically binding to the antigenic peptide. The invention further provides antibody-drug conjugates (ADCs) comprising the masked antibodies as well as pharmaceutical compositions comprising the masked antibodies or ADCs.

### FIELD OF THE INVENTION

The present invention relates to novel masked antibodies or antigen binding fragments thereof, comprising at least one light (LC) and heavy chain (HC) variable domain pair provided with a mask, said mask comprising two peptidic masking moieties specifically binding to each other, linked to the N-termini of the light and heavy chain, respectively, wherein at least one masking moiety is linked via a cleavable linker. The invention further provides antibody-drug conjugates (ADCs) comprising the masked antibodies as well as pharmaceutical compositions comprising the masked antibodies or ADCs.

### BACKGROUND OF THE PRESENT INVENTION

Antibodies are widely used in therapeutics to treat diseases like cancer, autoimmune diseases or inflammatory diseases. Antibodies specifically recognize and bind to a target antigen, which may trigger a therapeutic effect. For example, antibodies may specifically bind to receptors that are crucial in biological processes and may have an agonistic or antagonistic effect on the antigen-receptor. Receptor binding may interfere with, for example, cell growth (e.g., tumor growth) or may have an immunomodulating effect, e.g., triggering the immune system. Antibodies may have a therapeutic effect of their own and/or may be used as a carrier for drug delivery to a target site (e.g., a cancer cell), for example as part of an ADC.

In all instances the therapeutic effect hinges on the ability of an antibody to bind to its target antigen. Therapeutic antibodies should bind to their antigen with sufficient affinity and specificity to ensure the desired therapeutic effect can be achieved with minimal side-effects and/or toxicity. Not only the selectivity of the antibody for its target is important, but also the specificity of the target for the disease (site) to be treated. Some target antigens may also be present on non-target cells, for example in healthy tissue, and binding of the antibody at these sites should be minimized to avoid unwanted side-effects of treatment.

To avoid binding of therapeutic antibodies at sites other than the disease site, antibodies can be modified in such a way that their binding sites are “masked” and binding of the antibody to its target antigen is inhibited by the mask until the antibody reaches the desired target site. Only at the target site the ability of the antibody to bind to its target antigen is activated, by removal of the “mask”. To ensure that the mask is only removed at the desired target site (e.g., a diseased cell of a tumor), the component(s) of the mask (masking moieties) may be bound to the antibody by a cleavable linker that will only be cleaved when the antibody reaches its target destination. Cleavable linkers may comprise cleavage sites for enzymes specific for certain tissues (e.g., a cleavage site recognized by tumor-specific proteolytic enzymes such as tumor-specific matrix metalloproteinases found in the extracellular matrix (e.g., MMP2, MMP9, or MMP14), matriptase and urokinase plasminogen activator).

By cleavage of the cleavable linker(s) at the target site (e.g., a tumor site) the mask is removed from the antibody and the antigen binding site of the antibody becomes available for binding to its target antigen.

Several antibody masking technologies are known in the art and were reviewed in, for example, Lucchi et al., 2021, ACS Cent. Sci., 7, 724-738 and Lin et al., 2020, Journal of Biomedical Science, 27:76.

“Affinity-based” masking strategies known in the art rely on the use of a masking moiety which binds to the antigen binding site of an antibody, thereby preventing the masked antibody from binding to its target antigen until the mask is removed by cleavage of a cleavable linker used to link the masking moiety to the antibody. Such masking moieties are designed to bind to the antigen binding site of the masked antibody with lower affinity than the actual target antigen of the antibody. Once the linker holding the masking moiety and the antibody together is cleaved, the masking moiety is replaced by the target antigen, because the antigen binds to the targeting antibody with a higher affinity than the masking moiety.

An affinity-based masking method is described in WO2004/009638 (ISIS Innovation Ltd.). In the described method therapeutic antibodies are provided with a masking moiety which may be a peptide mimotope, reversibly bound to the antigen binding site of the antibody (referred to as the antibody combining site (ACS)), in the initial period after administration. The masking moiety is linked to the antibody heavy or light chain by an enzymatically cleavable linker (a linker which carries an enzyme-degradable motif, susceptible to cleavage by host enzymes in vivo). In WO2009/025846 (Cytomx, Univ. California), masked anti-VEGF antibodies are described, wherein the masking moiety is a peptide carrying a cysteine residue, bound to the antibody by a MMP2 protease cleavable peptide linker. The cysteine in the masking moiety forms a disulfide bridge with a cysteine near (or within) the target binding site of the antibody, thereby masking the antigen binding site of the antibody. In the cleaved state the antigen binding site of the antibody becomes available for binding to the target protein. In U.S. Pat. No. 8,895,702 B2 (Williams et al.), a pair of cross-masked antibodies is described, wherein a cross masking epitope binding to an antigen binding site of the other antibody is linked to each antibody by a cleavable linker, thus forming a cross-masked complex until the cleavable linkers are cleaved by MMP9 at the target site. Affinity-based masking strategies, where a masking moiety specifically binds to the antigen binding site of an antibody, have the disadvantage that each antibody requires a different specific mask for its own unique antigen binding site and requires careful optimization of the affinity of the mask for the antigen binding site.

Antibody masking technologies that provide more generally applicable masks that can be applied to different antibodies, irrespective of their target antigen, have also been developed. In these methods an antibody is provided with one or more masking moieties that do not act by directly binding to the antigen binding site of the antibody. Instead an antibody is provided with one or more masking moieties that prevent the antibody from binding to its target antigen by sterically interfering with the binding of the antibody rather than by occupation of the antigen binding site of the antibody. In these methods, N-termini of both the antibody light chain (LC) and antibody heavy chain (HC) of an LC-HC pair may be elongated with peptidic masking moieties that have an affinity towards each other and form a mask by binding to each other rather than to the antigen binding site of the antibody. These masking moieties may be bound to the N-termini of the antibody LC and HC by cleavable linkers in such a way that in the uncleaved state the two masking moieties bind to each other to form a mask shielding the antigen binding site of the antibody, while in the cleaved state the mask is removed and the antibody binding sites become available for binding to its target antigen again.

In WO2014/193973 (DCB USA LLC, Univ. Kaohsiung Medical), an antibody masking method is described, wherein the hinge region of a human antibody is used as a masking moiety. The blocking hinge region consists of two peptide arms interconnected by disulfide bonds. Each arm of the hinge region is connected to the heavy and light chain of an antibody binding domain, respectively, with cleavable linkers (four arms and four cleavable linkers per antibody). In WO2020/229553 (Ultrahuman Six Ltd.), a masking method is described wherein two antigen binding moieties, which both may be (parts of) antibodies binding to disease-specific antigens, are linked to each other by a cleavable peptide linker which comprises an amino acid sequence from a human hinge region. In the uncleaved state the binding of the moieties to their respective target antigens is blocked. In WO2018/107125 (Seattle Genetics Inc.), the use of coiled-coil forming peptides as masking moieties is described. The coiled-coil forming peptides are linked to the N-termini of at least one of the light and heavy chain pairs of an antibody via linkers comprising a protease cleavage site. In the uncleaved state the peptides associate to form a coiled coil structure and thereby reduce the binding affinity of the light-heavy chain pair of the masked antibody to a target antigen. On exposure to tumor-associated proteases, such as MMP2 and MMP9, the coiled-coil peptides are cleaved off and the binding of the antibody to its target antigen is restored.

A problem of existing masking technologies may be that the inclusion of the mask in the antibody product may lead to the formation of high molecular weight (HMW) aggregates during production. HMW aggregates can be formed, for example, when masking moieties on different antibodies bind to each other rather than to their counterpart masking moiety on the same antibody. As a result, the yield of useful masked antibodies in production may be low and/or additional steps have to be taken to remove the HMW aggregates from the product. Excess aggregation might also lead to induction of anti-drug antibodies (ADAs).

For several of the masking methods known in the art, separate steps were suggested to resolve the issue of HMW aggregate formation. In WO2020/247572 and WO2020/247574 (Seattle Genetics Inc.), the problem with HMW aggregate formation during purification, processing and storage in relation to a masking method using (hydrophobic) coiled coil masks is described. For example, it is suggested to store the masked antibodies in an aqueous buffer with a pH between 3.5 and 4.5. In WO2020/028401 (Amgen Inc.), the solution for the formation of HMW aggregates in the context of the production of masked antibodies is also sought in altering the pH of the pharmaceutical antibody composition.

Despite the fact that several universal masking technologies already exist, there still is a need for generally applicable antibody masking technologies that are easy to produce (e.g., with no or limited HMW aggregate formation). This is especially true for antibodies used in antibody-drug conjugates (ADC), where the antibody mask should also not interfere with the linking of linker-drug molecules to a masked antibody. Linker-drug molecules are often linked through (engineered) cysteines within the antibody protein molecule. In particular, masking technologies relying on peptide masking moieties binding to or through the formation of —S—S— bridges between cysteines may be less suitable for use in ADCs based on masked antibodies, because of the risk of interference with linker-drug conjugation.

### SUMMARY OF THE PRESENT INVENTION

The present invention relates to a novel way of masking antibodies. The present invention provides a masked antibody or antigen binding fragment thereof, comprising at least one light and heavy chain pair provided with a mask, said mask comprising two peptidic masking moieties specifically binding to each other, linked to the N-termini of the light and heavy chain of the pair, respectively, wherein at least one masking moiety is linked via a cleavable linker, one of the two masking moieties comprises an antigenic peptide and the other comprises a single domain antibody (sdAb) specifically binding to the antigenic peptide.

The present invention is based on the novel use of an antigenic peptide and sdAb specifically binding to said antigenic peptide in masking moieties in a masked antibody.

The present invention further provides antibody-drug conjugates (ADCs) based on the masked antibodies according to the invention, the use of said masked antibodies or ADCs as a medicament as well as pharmaceutical compositions comprising said masked antibodies or ADCs.

More specifically, the invention is set out as follows.

An activatable antibody or antigen binding fragment thereof, comprising an antigen binding site, comprising a heavy chain variable domain and a light chain variable domain capable of binding to a target protein, wherein said antigen binding site is provided with a mask suitable for inhibiting binding of the antigen binding site to the target protein, the mask comprising two masking moieties:
i. a masking moiety linked to the N-terminus of the variable heavy chain domain, and
ii. a masking moiety linked to the N-terminus of the variable light chain domain, wherein at least one of the masking moieties is linked through a cleavable linker, and wherein one masking moiety comprises an antigenic peptide and the other masking moiety comprises a single domain antibody (sdAb) specifically binding to the antigenic peptide.

Activatable antibodies according to the invention may be used in therapy, by administering it to a subject in need of treatment (e.g. a cancer patient receiving immunotherapy with a therapeutic anti-cancer antibody or antibody drug conjugate (ADC).

An activatable antibody or antigen binding fragment thereof, according to the invention comprises an antigen binding site, capable of binding to a target protein in a subject. After administering the antibody to a subject, the mask is removed in vivo, preferably at the target (disease, e.g. tumor) site, by cleavage of at least one cleavable linker.

In a preferred embodiment, the activatable antibody or antigen binding fragment thereof comprises two antigen binding sites, wherein both antigen binding sites are provided with a mask.

Alternatively or in combination with an earlier preferred embodiment, in a preferred embodiment, the antigenic peptide is 4-20 amino acid residues in length, preferably wherein the antigenic peptide is 12-20 amino acid residues in length and comprises or consists of an amino acid sequence as defined in SEQ ID NO: 51. More preferably, the antigenic peptide comprises or consists of an amino acid sequence as defined in SEQ ID NO: 1, SEQ ID NO: 2 (SA mutant) or SEQ ID NO: 3 (ST mutant). Even more preferably, the antigenic peptide consists of an amino acid sequence as defined in SEQ ID NO: 1, SEQ ID NO: 2 (SA mutant) or SEQ ID NO: 3 (ST mutant).

Alternatively or in combination with an earlier preferred embodiment, in a preferred embodiment, the nanobody comprises or consists of the amino acid sequence as defined in SEQ ID NO: 4, a humanized version thereof, or a variant of SEQ ID NO: 4, such as the variant depicted in SEQ ID NO: 76 (RA mutant), SEQ ID NO: 77 (RE mutant), or SEQ ID NO: 78 (RS mutant), or a humanized version of said variant. In the RA, RE and RS mutant of the B2C nanobody, the original CDR3 (SEQ ID NO: 5), is replaced with the CDR3 depicted in SEQ ID NO: 6, SEQ ID NO: 7, or SEQ ID NO: 8 respectively.

A humanized version of the BC2 nanobody may be a nanobody with an amino acid sequence depicted in SEQ ID NO: 79 (BC2_{Nb humanized 1}), SEQ ID NO: 80 (BC2_{Nb humanized 2}) or SEQ ID NO: 81 (BC2_{Nb humanized 3}).

A humanized version of the RA mutant nanobody may be a nanobody with an amino acid sequence depicted in SEQ ID NO: 82 (RAₕ₁), SEQ ID NO: 83 (RAₕ₂) or SEQ ID NO: 84 (RAₕ₃). A humanized version of the RE mutant nanobody may be a nanobody with an amino acid sequence depicted in SEQ ID NO: 85 (REₕ₁), SEQ ID NO: 86 (REₕ₂) or SEQ ID NO: 87 (REₕ₃). A humanized version of the RS mutant nanobody may be a nanobody with an amino acid sequence depicted in SEQ ID NO: 88 (RSₕ₁), SEQ ID NO: 89 (RSₕ₂) or SEQ ID NO: 90 (RSₕ₃).

In a preferred embodiment, the antigenic peptide comprises or consists of a peptide with the amino acid sequence defined in SEQ ID NO: 2 (SA mutant) or a humanized version thereof, and the nanobody comprises the amino acid sequence as defined in SEQ ID NO: 78 (RS mutant).

Preferably a humanized nanobody is RSₕ₁.

Alternatively or in combination with an earlier preferred embodiment, in a preferred embodiment, the masking moiety comprising the sdAb is linked to the N-terminus of the light chain variable domain and the masking moiety comprising the antigenic peptide is linked to the N-terminus of the heavy chain variable domain.

In a preferred embodiment, the antigenic peptide is preceded by a spacer amino acid sequence as defined in SEQ ID NO: 9, connected to the N-terminus of the antigenic peptide.

Alternatively or in combination with an earlier preferred embodiment, in a preferred embodiment, the cleavable linker comprises one or more cleavage sites recognized by one or more tumor specific proteases. This means that each cleavable linker may contain one or more cleavage sites. Each cleavage site may be recognized by only one, or by multiple tumor specific proteases. Preferably at least one cleavable linker comprises a cleavage site recognized by matriptase or a cleavage site recognized by a metalloproteinase.

In a second aspect, the present invention relates to an antibody-drug conjugate (ADC), comprising the activatable antibody or antigen binding fragment thereof according to the invention and a linker-drug.

In a third aspect, the present invention relates to a pharmaceutical composition comprising an activatable antibody or antigen binding fragment thereof according to the invention, or an ADC according to the invention, and a pharmaceutically acceptable excipient.

In a fourth aspect, the present invention relates to an activatable antibody or antigen binding fragment thereof according to the invention, an ADC according to the invention or a pharmaceutical composition according to the invention for use as a medicament.

In a fifth aspect, the present invention relates to an activatable antibody or antigen binding fragment thereof according to the invention, an ADC according to the invention or a pharmaceutical composition according to the invention for use in the treatment of cancer, an autoimmune disease or an infectious disease, preferably for use in the treatment of cancer.

In a sixth aspect, the present invention relates to a nucleic acid construct comprising: (a) a nucleotide sequence encoding a heavy chain variable domain and a masked moiety; and a nucleotide sequence encoding a light chain variable domain and a masked moiety; wherein the nucleotide sequences are operably linked to an expression control sequence for expression in a host cell, preferably a mammalian host cell. Preferably, the heavy chain variable domain, light chain variable domain and masked moieties are as defined in relation to the first aspect of the invention. In a preferred embodiment, the nucleic acid construct is a vector compatible with the host cell.

In a seventh aspect, the present invention relates to a host cell comprising a nucleic acid construct of the invention.

In an eighth aspect, the present invention relates to a method for producing an activatable antibody or antigen binding fragment thereof according to the invention, the method comprising the step of culturing a host cell according to the invention under conditions conducive to expression of the activatable antibody or antigen binding fragment thereof.

In a ninth aspect, the present invention relates to a use of an antigenic peptide and a sdAb specifically binding to the antigenic peptide as part of a mask in an activatable antibody or an antigen binding fragment thereof.

### BRIEF DESCRIPTION OF THE DRAWINGS

FIG. 1A: Schematic representation of antibodies with a masking moiety comprising a nanobody on the HC and a masking moiety comprising an antigenic peptide on the LC and vice versa.

FIG. 1B: Schematic representation of the formation of High Molecular Weight (HMW) complexes between masked antibodies during production.

FIG. 2A-L: Dose-dependent binding of antibody S103, and antibodies S201-211 when masked and demasked after MMP2 treatment on HER2-positive SK-BR-3 cells, compared to unmasked (naked) trastuzumab binding, as measured on a flow cytometer. Data show the MFI (median fluorescence intensity) from one experiment.

FIG. 3: Cytotoxicity on HER2-positive SK-BR-3 cells induced by ADC-S105, when it is masked and after MMP2 treatment (demasked) compared to trastuzumab ADC (ADC-TRA41C) and isotype control rituximab ADC (ADC-RIT41C).

FIG. 4: Cytotoxicity induced by ADCs ADC-S423 (masking moieties linked via L9-linker which is cleavable by MMP2 proteases), ADC-S426 (masking moieties linked via L13 scrambled linker which is not cleavable by MMP2 proteases), ADC-TRA41C, and isotype control ADC-RIT41C on HER2-positive SK-BR-3 cells. As a control for the effect of MMP2 on the ADC, ADC-TRA41C was also treated with MMP2 proteases.

FIG. 5: Dose-dependent binding on HER2-positive SK-BR-3 cells measured on a flow cytometer for ADC-S423 and ADC-S426, when it is masked and after MMP2 treatment compared to ADC-TRA41C binding (+/− MMP2-treated). Data show the MFI (median fluorescence intensity) from one experiment.

FIG. 6: Dose-dependent binding of half-masked antibodies S109 and S110 compared to trastuzumab on HER2-positive SK-BR-3 cellsHER2-, as measured on a flow cytometer. Data show the MFI (median fluorescence intensity) from two experiments.

FIG. 7A: Dose-dependent binding of ADC-S424 and ADC-S425, when masked and half-demasked after MMP2 treatment compared to ADC-TRA41C on HER2-positive SK-BR-3 cells. Data show the MFI (median fluorescence intensity) from one experiment.

FIG. 7B: Cytotoxicity induced by half-masked ADCs ADC-S109, ADC-S 110 and ADC-S111 compared to cytotoxicity induced by ADC-TRA41C and isotype control ADC-RIT41C on HER2-positive SK-BR-3 cells.

FIG. 7C: Cytotoxicity induced by half-masked ADCs ADC-S424, ADC-S425 and compared to cytotoxicity induced by ADC-TRA41C and isotype control ADC-RIT41C on HER2-positive SK-BR-3 cells.

FIG. 8A-F: Results of target binding of naked (unmasked), masked and masked-41C versions of mAbs targeting EpCAM (ING-1 and adecatumumab), CD137 (4-1BB) (urelumab), and TROP2 (sacituzumab), Tissue Factor (MORAb-066 and tisotumab) and naked (unmasked), masked CD137 (4-1BB) (urelumab), as measured using biolayer interferometry and corrected for differences in concentration (target binding/protein A binding*1000).

FIG. 9: Percentage high molecular weight (HMW (%)-Y-axis) versus average Drug-to-Antibody Ratio DAR-X-axis) for ADCs with varying DAR produced from masked antibodies S103 and S211.

FIG. 10: Results of testing a linker matrix of (18×18=324) linker pairs to attach a mask to the tisotumab antibody. The masking moieties used in each case were a pair of masking moieties comprising a masking moiety with the BC2T SA mutant peptide linked to each LC, and a masking moiety with the BC2-Nb RS mutant nanobody linked to each HC of the anti-TF antibody tisotumab. For all antibodies produced the amount of antibody produced (μg/mL) and the HMW (%) (percentage of total antibody lost due to formation of high molecular weight complexes) were determined. The obtained amount of antigen for each antibody is reflected in FIG. 10A. The HMW (%) for each antibody is reflected in FIG. 10B. The results of a Bio-layer interferometry (BLI) binding experiment are reflected as binding to tissue factor/binding to protein A*1000 in FIG. 10C. “ND” means no value could be determined.

FIG. 11: Results of in vivo testing of ADC 1-13 in the non-small cell lung cancer model LXFA629. ADCs dosed with 2.4 mg/kg. The unmasked (naked) Tisotumab P41C and ADC (ADC13) were dosed at 2 mg/kg. Arrow indicates moment of randomization and dosing, N=6 mice/group, data are presented as the mean±SEM. every data point represents the average value of the absolute tumor volumes (mm³) for all mice in each group.

FIG. 12: Absolute tumor volume (mm³) of TF-positive LXFA629 tumor bearing mice in each group on the last day (day 42) including mean.

### DETAILED DESCRIPTION OF THE PRESENT INVENTION

With the present invention a new antibody masking method is provided, wherein an antigenic peptide and a single domain antibody (sdAb) specifically binding to the antigenic peptide are used as part of masking moieties.

The present invention provides a masked antibody or antigen binding fragment thereof, comprising at least one light and heavy chain pair provided with a mask, said mask comprising two peptidic masking moieties specifically binding to each other, linked to the N-termini of the light and heavy chain of the pair, respectively, wherein at least one masking moiety is linked via a cleavable linker, one of the two masking moieties comprises an antigenic peptide and the other comprises a sdAb specifically binding to the antigenic peptide.

The invention thus provides a new use of an antigenic peptide and sdAb specifically binding to the antigenic peptide as part of masking moieties in a masked antibody or antigen binding fragment.

The use of a sdAb in/as an antibody masking moiety has several advantages. The masking effect is based on the specific affinity between the sdAb and its antigenic peptide, binding partner. By binding to each other the two masking moieties shield the binding site of the antibody and block, or at least significantly reduce, its ability to bind to its target antigen.

The bond between the masking moieties in a mask provided with the present invention does not rely on the formation of any —S—S— bridges formed between cysteine residues in both masking moieties. Especially when the masked antibody is part of an ADC, this is an advantage, since linker-drugs are often bound to (engineered) cysteine residues in the antibody protein. Masked antibodies or masked antigen binding fragments thereof according to the invention are thus particularly useful in ADCs. Masked antibodies according to the invention further have the advantage that they can be produced at relatively high titer without losing a substantial part of the produced masked antibody due to the formation of high molecular weight (HMW) complexes.

Thus, in a first aspect, the present invention relates to an activatable antibody or antigen binding fragment thereof, comprising an antigen binding site, comprising a heavy chain variable domain and a light chain variable domain capable of binding to a target protein wherein said antigen binding site is provided with a mask suitable for inhibiting binding of the antigen binding site to the target protein, the mask comprising two masking moieties:
i. a masking moiety linked to the N-terminus of the variable heavy chain domain, and
ii. a masking moiety linked to the N-terminus of the variable light chain domain, wherein at least one of the masking moieties is linked through a cleavable linker, and wherein one masking moiety comprises an antigenic peptide and the other masking moiety comprises a single domain antibody (sdAb) specifically binding to the antigenic peptide.

Activatable antibodies according to the invention may be used in therapy, by administering it to a subject in need of treatment (e.g. a cancer patient receiving immunotherapy with a therapeutic anti-cancer antibody or antibody drug conjugate (ADC).

An activatable antibody or antigen binding fragment thereof, according to the invention comprises an antigen binding site, capable of binding to a target protein in a subject. In the activatable antibody the antigen binding site is provided with a mask that inhibits binding to the target protein. After administering the antibody to a subject, the mask is removed in vivo, preferably at the target (disease, e.g. tumor) site, where at least one cleavable linker is cleaved, after which the binding site of the antibody can bind to the target protein in the subject.

### Antibody

The term “antibody” as used herein preferably refers to an antibody comprising two heavy chains and two light chains. The antibodies to be used in accordance with the invention may be of any isotype such as IgA, IgE, IgG, or IgM antibodies. Preferably, the antibody is an IgG antibody, more preferably an IgG₁ or IgG₂ antibody. The antibodies may be chimeric, humanized or human. Preferably, the antibodies are humanized or human. Even more preferably, the antibody is a humanized or human IgG antibody, more preferably a humanized or human IgG₁ monoclonal antibody. The antibody may have κ (kappa) or λ (lambda) light chains, preferably κ (kappa) light chains, i.e., a humanized or human IgG_{1-κ} antibody.

The term “antigen binding fragment” as used herein includes a Fab, Fab′, F(ab′)₂, Fv, scFv or reduced IgG (rIgG) fragment, or any fragment of an antibody comprising at least the variable binding region of a light (LC) and heavy chain (HC) pair.

“Humanized” forms of non-human (e.g., rodent) antibodies are antibodies (e.g., non-human-human chimeric antibodies) that contain minimal sequences derived from the non-human antibody. Various methods for humanizing non-human antibodies are known in the art. For example, the antigen-binding complementarity determining regions (CDRs) in the variable domains (also termed variable regions or VRs) of the heavy chain (HC) and light chain (LC) are derived from antibodies from a non-human species, commonly mouse, rat or rabbit. These non-human CDRs may be combined with human framework regions (FRs, i.e., FR1, FR2, FR3 and FR4) of the variable regions of the HC and LC, in such a way that the functional properties of the antibodies, such as binding affinity and specificity, are at least partially retained. Selected amino acids in the human FRs may be exchanged for the corresponding original non-human species amino acids to further refine antibody performance, such as to improve binding affinity, while retaining low immunogenicity. The thus humanized variable regions are typically combined with human constant regions. An exemplary method for humanization of non-human antibodies is the method of Winter and co-workers (Jones et al, 1986, Nature, 321, 522-525; Riechmann et al, 1988, Nature, 332, 323-327; Verhoeyen et al, 1988, Science, 239, 1534-1536). Alternatively, non-human antibodies can be humanized by modifying their amino acid sequence to increase similarity to antibody variants produced naturally in humans. For example, selected amino acids of the original non-human species FRs are exchanged for their corresponding human amino acids to reduce immunogenicity, while retaining the antibody's binding affinity. For further details, see Jones et al, 1986, Nature, 321, 522-525; Riechmann et al, 1988, Nature, 332, 323-327; and Presta, 1992, Curr. Op. Struct. Biol., 2, 593-596. See also the following review articles and references cited therein: Vaswani and Hamilton, 1998, Ann. Allergy, Asthma and Immunol., 1, 105-115; Harris, 1995, Biochem. Soc. Transactions, 23, 1035-1038; and Hurle and Gross, 1994, Curr. Op. Biotech., 5, 428-433.

The CDRs may be determined using the approach of Kabat (in Kabat, E. A. et al, 1991, Sequences of Proteins of Immunological Interest, 5^{th} Ed. Public Health Service, National Institutes of Health, Bethesda, MD, NIH publication no. 91-3242, pp. 662, 680, 689), Chothia (Chothia et al, 1989, Nature 342, 877-883) or IMGT (Lefranc, 1999, The Immunologist, 7, 132-136).

Typically, the antibody is a monospecific (i.e., specific for one antigen; such antigen may be common between species or have similar amino acid sequences between species) or bispecific (i.e., specific for two different antigens of a species) antibody comprising at least one HC and LC variable region binding to an antigen target, preferably a membrane bound antigen target which may be internalizing or not internalizing. Preferably, in the case of an antibody drug conjugate, the antibody is internalized by the target cell after binding to the (antigen) target, after which an active effector molecule is released intracellularly.

Examples of therapeutic antibodies known in the art include blinatumomab (CD19), rituximab (CD20), epratuzumab (CD22), iratumumab and brentuximab (CD30), vadastuximab (CD33), tetulumab (CD37), isatuximab (CD38), bivatuzumab (CD44), lorvotuzumab (CD56), vorsetuzumab (CD70), milatuzumab (CD74), polatuzumab (CD79), rovalpituzumab (DLL3), futuximab (EGFR), oportuzumab (EPCAM), ING-1 (EpCAM), adecatumumab (EpCAM), farletuzumab (FOLR1), glembatumumab (GPNMB), trastuzumab, pertuzumab and margetuximab (HER2), etaracizumab (integrin), anetumab (mesothelin), pankomab (MUC1), enfortumab (Nectin-4), H8, A1, A3 (5T4), MORAb-66, tisotumab (TF) sacituzumab (TROP2) and urelumab (TNFRSF9, CD137), etc.

### Masked Antibody or Antigen Binding Fragment

A masked antibody or antigen binding fragment thereof is an antibody or antigen binding fragment as defined herein above, provided with a (removable) mask. The antibody or antigen binding fragment mask consists of one or more masking moieties and blocks, or at least significantly reduces, the ability of the antibody or antigen binding fragment to bind to its target antigen, until the blocking effect of the mask is removed (“demasking”) and the capacity of the antibody or antigen binding fragment to bind to its target antigen is sufficiently restored to have the desired therapeutic effect. Masked antibodies or antigen binding fragments are also referred to as activatable antibodies or antigen binding fragments, or antibody or antigen binding fragment-prodrugs, to indicate that they have to be activated, by removal of the masking effect of the antibody mask, to display their therapeutic effect. The terms “masked antibody” and “activatable antibody” are used interchangeable herein. Masking moieties used to create at least one “mask” in masked antibodies or antigen binding fragments according to the invention come as a pair; one masking moiety comprises an antigenic peptide, while the other comprises a sdAb specifically binding to the antigenic peptide. One of the masking moieties of the pair is linked to the HCVR while the other is linked to the LCVR of at least one HC/LC binding pair of a masked antibody or antigen binding fragment thereof, according to the invention. By binding to each other the masking moieties form a mask that shields the (at least one) binding site of the antibody or antigen binding fragment thereof. An antibody, or antigen binding fragment thereof, with two antigen binding sites can thus carry two masks; one on each HCVR/LCVR pair.

Antibodies or antigen binding fragments thereof can be masked to prevent or reduce off-target effects, because most target antigens are not completely specific to the target (disease) site. The mask reduces the capabilities of the antibody or antigen binding fragment thereof to bind to its target antigen, by shielding the antigen binding site(s) in the masked antibody or antigen binding fragment until it reaches its target (disease) site where the masking effect is removed. With reduced binding is meant that the binding capacity of the masked antibody or antigen binding fragment for its target is significantly reduced by the mask, when compared to the naked (unmasked) or demasked (after the mask has been removed from the antigen binding site) antibody or antigen binding fragment.

### Antibody or Antigen Binding Fragment Binding

Therapeutic antibodies or antigen binding fragments are selected for their selectivity as well their affinity towards a target antigen. Affinity is the strength of the binding interaction between a binding molecule and its ligand (e.g., between an antibody or antigen binding fragment and its target antigen). A target antigen can be an antigenic (part of a) protein, referred to as an “antigenic determinant” or “epitope”, that may be presented on the surface of a target cell to which the antibody or antigen binding fragment thus binds. The target cell may be a tumor cell in case of therapeutic antibodies or antigen binding fragments used in the treatment of cancer.

Antibody or antigen binding fragment selectivity is a measure of how well an antibody or antigen binding fragment binds to its target antigen in a mixture of different proteins. In other words: if the epitope to which the antibody or antigen binding fragment binds is unique to one particular target antigen, selectivity is high because the antibody or antigen binding fragment exclusively binds to the target antigen and does not cross-react with any off-target protein present in the mixture.

Antibody or antigen binding fragment specificity represents the ability of the antibody or antigen binding fragment to recognize a single epitope. A monoclonal antibody or antigen binding fragment, binding to a single epitope, is thus specific. But when the single epitope to which the antibody or antigen binding fragment binds appears in multiple proteins, the antibody or antigen binding fragment may be specific (for the epitope to which it binds with high affinity) but not selective (since the epitope occurs in non-target proteins as well). Therapeutic antibodies or antigen binding fragments preferably bind to their target antigen with high affinity and selectivity.

Antibody or antigen binding fragment binding to a target antigen can be measured and compared by a variety of techniques, wherein a signal measured, represented by a particular parameter, is a reflection of the degree of binding of the antibody or antigen binding fragment to its target antigen. To investigate a reduction in binding to a target antigen due to masking of the antigen binding site of an antibody or antigen binding fragment, the binding signal for a “naked” antibody or antigen binding fragment (an unmasked antibody or antigen binding fragment) can be compared to the binding signal of the same antibody or antigen binding fragment provided with a mask (masked antibody or antigen binding fragment) under the same circumstances (e.g., at the same concentration). A difference in an obtained signal between the naked and masked antibody or antigen binding fragment, a reduced signal for the masked antibody or antigen binding fragment, is an indication that the masked antibody or antigen binding fragment shows reduced binding to the target antigen, when compared to the naked antibody or antigen binding fragment. In the same way recovery of binding can be determined by comparing a signal measured for a masked antibody or antigen binding fragment with a binding signal obtained for antibodies or antigen binding fragments where the masking effect has been removed (“demasked” antibody or antigen binding fragment). To investigate binding recovery after demasking, a signal for a demasked antibody or antigen binding fragment can be also compared with a signal obtained for a naked antibody or antigen binding fragment.

One parameter to express antibody or antigen binding fragment binding or affinity is the dissociation constant (K_{D}). For antibodies or antigen binding fragments, K_{D} is the ratio of the antibody or antigen binding fragment dissociation rate (K_{off}) to the antibody or antigen binding fragment association rate (Kₒₙ), or as the multiplied concentrations of unbound over bound; [A]*[B]/[AB] at equilibrium, where the association and dissociation rate of the binding partners are the same. The higher the concentration of the binding partners, the more likely it is that they bind to each other. Binding partners with a lower K_{D}, will still be able to bind at lower concentrations, whereas binding partners with a high K_{D} will need a high concentration to still be able to bind to each other. The smaller the K_{D} value, the greater the binding affinity between the two binding partners. K_{D} is expressed as a concentration in Molar units. Most antibodies or antigen binding fragments have K_{D} values in the low micromolar (10⁻⁶) to nanomolar (10⁻⁷ to 10⁻⁹) range. High affinity antibodies or antigen binding fragments are generally considered to be in the low nanomolar range (10⁻⁹) with very high affinity antibodies or antigen binding fragments being in the picomolar (10⁻¹) to low nanomolar range. K_{D} is influenced by various factors, for example by buffer composition, pH and temperature. Affinity (K_{D}) can be measured, for example, with Surface Plasmon Resonance (SPR).

A technique suitable for an indicative screening of antibody or antigen binding fragment binding to (immobilized) target antigen is Biolayer interferometry (BLI). BLI is an optical technique whereby interference patterns of white light, reflected from the tips of sensors coated with a biolayer, as well as from an internal reference layer are measured; a sensor tip with immobilized isolated target antigen may be contacted with a solution with a pre-determined concentration of an antibody or antigen binding fragment. When the antibody or antigen binding fragment in solution binds to the immobilized target antigen on the sensor tips, the optical thickness (number of bound molecules) of the biolayer on the sensor tip changes, causing a wavelength shift in the interference patterns of the reflected light, measured in real time. The measured wavelength shift is a direct measure of the optical thickness of the biolayer on the sensor tip (expressed in nm) and is a reflection of the degree of binding of the antibody or antigen binding fragment to the (immobilized) antigen. BLI can be used, for example, to confirm that antibodies are adequately masked; binding of masked antibodies or antigen binding fragments to their immobilized target antigen can be compared to binding of unmasked or demasked antibodies or antigen binding fragments, and a first indication of the effectivity of the mask can be obtained. In such screenings a significant signal shift was seen for all masked antibodies according to the invention that were tested, against a variety of target antigens, when the signal of naked (unmasked) and masked antibodies were compared.

Binding of antibodies or antigen binding fragments to target cells (over)expressing the target antigen recognized by the antibody or antigen binding fragment can be measured in vitro in a flow cytometer. Target cells expressing target antigen may be contacted with different concentrations of (masked) antibodies, antigen binding fragments, or ADCs according to the invention, and stained with secondary, fluorescently labelled antibodies binding to the first antibodies or antigen binding fragments. The read out will provide the Median Fluorescent Intensity (MFI), which again is a measure for antibody or antigen binding fragment binding to its target antigen expressed on target cells. Plotting the MFI against different antibody or antigen binding fragment concentrations up to, and passed concentrations where maximal binding (saturation) occurs, yields a dose-response curve (DRC). From a dose-response curve an EC₅₀ value (the concentration of an antibody or antigen binding fragment that gives half-maximal response) can be deduced. The EC₅₀ shift (which may be expressed as the ratio between EC₅₀ of masked and naked (unmasked) antibodies or antigen binding fragments), is a measure for the reduction in target binding due to masking, provided complete dose-response curves can be obtained for masked antibodies or antigen binding fragments. By comparing the EC₅₀ of demasked antibodies or antigen binding fragments (antibodies or antigen binding fragments from which the mask was removed) with the EC₅₀ of naked (unmasked) antibodies or antigen binding fragments, the degree of recovery of binding after demasking can be determined. Usually EC₅₀ values are determined by curve fitting with software such as GraphPad Prism (www.graphpad.com). Using the same software tool, the ratio between two EC₅₀ values can also be determined (the EC₅₀ shift or potency shift).

Masked antibodies or antigen binding fragments, hardly bind to their target (which already proves that they are efficiently masked). As a consequence, saturated binding may not always be reached in binding experiments, even at relatively high concentrations, which may complicate EC₅₀ determinations.

An alternative for EC₅₀ determinations is comparing the concentrations where minimal binding starts to occur. For masked antibodies according to the invention, the concentration where minimal binding could be observed was significantly higher than for unmasked or demasked antibodies.

For example, for masked antibodies (e.g., based on trastuzumab, as shown in the experiments reflected in the Examples) according to the invention, when tested for binding on antigen-positive cells by flow cytometry, minimal binding was only observed at very high concentrations above 3 μg/mL, and often in the range from 10-30 μg/mL or higher, while minimal binding for naked or demasked antibodies was already observed at 0.04 μg/mL.

Binding is preferably reduced by at least 10 fold, preferably 50-100 fold, or even 100-500 fold or above 500 fold. As a consequence of the reduced binding of masked antibodies to their target antigen expressed on a target cell, cytotoxicity will be preferably be reduced by a similar factor.

The reduction in cytotoxicity on target cells, for a masked antibody, antigen binding fragment, or ADC according to the invention, in comparison to a naked or demasked antibody, antigen binding fragment, or ADC can be determined as well. When various masked ADCs according to the invention were tested for their cytotoxicity in comparison with ADCs based on naked antibodies very significant IC₅₀ shifts were observed. The IC₅₀ shift of the masked ADCs was over 30, and even a 500-fold shift was observed, when compared to the IC₅₀ of the same ADC based on the naked antibody. These findings represent very efficient masking of the binding regions of masked antibodies according to the invention.

What is also important is that, after demasking (i.e., removal of the mask) binding to the target, and consequently cytotoxicity, is restored to a sufficient level for the demasked antibody or antigen binding fragment to have the desired therapeutic effect. Preferably, the demasked antibody or antigen binding fragment binds to the target antigen as efficiently as the unmasked antibody or antigen binding fragment would. So if the unmasked antibody or antigen binding fragment binds to its target with an affinity in the high nanomolar range, preferably the demasked antibody or antigen binding fragment does so as well. Preferably demasked antibodies bind to their target antigen exactly like the corresponding naked antibody does. Some reduction in binding in a demasked antibody, when compared to the naked antibody can be tolerated. Preferably the order of binding of a demasked antibody, for example as expressed as an EC₅₀ shift, is in the order of 1-2, more preferred between 1 and 1.5, and of course most preferred close to 1 (meaning that the dose response curves for demasked and naked antibody virtually overlap). With antibodies according to the invention an almost full recovery of binding after demasking was found when compared to binding of the naked antibody. EC₅₀ shifts between unmasked and demasked antibodies were often in the range between 1-2, indicating that binding was restored after demasking. The same goes for the cytotoxicity of demasked antibodies; After demasking almost full recovery of induction of cytotoxicity was observed, when compared to naked antibodies.

In a masked antibody or antigen binding fragment according to the invention, the mask comprises a pair of peptidic masking moieties having a specific binding affinity towards each other, wherein one masking moiety of the pair comprises an antigenic peptide, while the other masking moiety comprises a single domain antibody (sdAb) specifically binding to the antigenic peptide. The masking moieties are linked to the N-termini of at least one HC and LC pair of the masked antibody or antigen binding fragment. The masked antibodies or antigen binding fragments according to the invention are effectively masked and they are effectively demasked once they reach their target site.

### Masking Moieties

Masking moieties are the actual (peptide) moieties (reversibly) bound to the antibody or antigen binding fragment thereof that, when conjugated to an antibody or antigen binding fragment can form a mask. Masked antibodies, or antigen binding fragments thereof, according to the invention are provided with a mask comprising a set of two peptide masking moieties linked to the N-termini of the heavy chain variable region (HCVR) and the light chain variable region (LCVR) that together form the antigen binding site. One of the two masking moieties of the pair of masking moieties comprises an antigenic peptide, while the other comprises a single domain antibody (sdAb) specifically binding to the antigenic peptide. At least one of the masking moieties is linked via a cleavable linker to the N-terminus of the HCVR and/or LCVR. By binding to each other, the masking moieties together form a mask, shielding the antigen binding site of the masked antibody or antigen binding fragment. When the antigen binding site of the antibody or antigen binding fragment is shielded by the mask, the ability of the masked antibody or antigen binding fragment to bind to its target antigen is significantly reduced. The antibody or antigen binding fragment can be demasked by cleavage of the at least one cleavable linker. The masking of the antibody or antigen binding fragment is thus reversible; the masking effect can be removed by cleavage of at least one cleavable linker. Demasking thus does not require breaking the bond between the sdAb and antigenic peptide, together making up the mask (although the masking moieties may not remain bound to each other once at least one linker has been cleaved).

An antigenic peptide for use in an antibody masking moiety in the present invention can be any suitable antigenic peptide as part of a sdAb/peptide pair. The peptide is selected to contain an epitope sequence (preferably a single epitope sequence) specifically recognized by the sdAb in the other masking moiety of a pair of masking moieties together forming a mask.

Preferably, selected antigenic peptide sequences for use in a masking moiety are relatively short (such as the peptide tags used in sdAb-based tagging technologies known in the art) so as not to hamper antibody production and allow for efficient masking and demasking. Typically, the length of such antigenic peptide sequences are in the order of 4-20, preferably 8-15 amino acids. More preferably, the antigenic peptide sequence has a length of 12-15 amino acid residues. Preferably, such peptides are derived from a human protein so as to minimize chances of immunogenicity of the antigenic peptide.

A single domain antibody (sdAb) is a (recombinant) antigen binding fragment having a unique structure composed of a single heavy chain with one variable domain only. The first developed sdAbs were V_{H}H fragment (variable domain) fragments derived from the heavy chain antibodies of a camelid (e.g., llamas, camels, dromedaries, Bactrian camels, alpacas, vicunas and guanacos). (S. Muyldermans et al., 2009, Veterinary Immunology and Immunopathology 128, 178-183). Heavy-chain antibodies circulating in camelids comprise heavy chains only and are naturally devoid of light chains. SdAbs derived from these camelid heavy-chain only antibodies are commonly referred to as “nanobodies”, or “V_{H}H” (Muyldermans S., 2013, annual 82:775-97. doi: 10.1146/annurev-biochem-063011-092449). The term “nanobody” was originally adopted by the Belgian company Ablynx, because these sdAbs have nanomolecular size (“NANOBODY” is a registered trademark of Ablynx N. V.) and the term nanobody is often used as the equivalent of sdAb, also encompassing sdAbs from other species. In addition to camelids, sdAbs have also been found in cartilaginous fishes (e.g., sharks). Sharks and other cartilaginous fish also produce heavy chain antibodies. These antibodies are called Ig new antigen receptors or IgNARs. IgNARs are composed of two identical heavy chains. The variable domain in these heavy chains is called a V-NAR domain. IgNARs also have a single variable domain. The V-NAR domain contains 2 CDRs, in contrast to 3 CDRs in the V_{H}H domain of camelid sdAbs (Cheong et al., 2020, International journal of biological macromolecules, 147, 369-375). sdAbs can also be obtained from common (human) IgG antibodies (Chen et al., 2009, Methods Mol Biol., 525: 81-99).

The sdAbs used in a masking moiety according to the invention may be humanized by methods known in the art (Vincke et al., 2009, The Journal of Biological Chemistry Vol. 284, No. 5, 3273-3284). SdAbs used in masking moieties in masked antibodies or antigen binding fragments according to the invention preferably are nanobodies or are a humanized version and/or variant thereof.

SdAbs can be selected and produced in a variety of ways known in the art. Methods to select nanobodies are reviewed in S. Muyldermans, 2021, The FEBS Journal, 288, 2084-2102; Hassanzadeh-Ghassabeh et al., 2013, Nanomedicine (Lond), 8(6):1013-26.

SdAbs are small (12-15 kD) compared to common antibodies (150-160 kD), which common antibodies are composed of two HC/LC pairs. SdAbs are also smaller than antigen binding fragments of common antibodies, such as Fab fragments, consisting of one (half) HC and an LC, with a molecular weight of about 50 kD. SdAbs are the smallest antibody fragments that still bind selectively to a specific target antigen.

SdAbs have been used for a variety of purposes, for example, in therapeutics (e.g., as a targeting moiety for a cytotoxic drug), but also in diagnostic and imaging applications where nanobodies were linked to tracers (de Meyer et al., 2014, Trends in Biotechnology, Vol. 32, No. 5, 263-270; Bao et al., 2021, EJNMMI Res 11:6, https://doi.org/10.1186/s13550-021-00750-5). For example, sdAbs against certain peptide tags were developed for use in immunoprecipitation of tagged proteins. Such tags include the tags developed by, for example, ChromoTek, Nanotag Biotechnologies, and others. Peptide-tag specific antibodies are also described by Ren et al., 2020, Journal of Chromatography A, 1624, 461227.

In a preferred embodiment of the invention, the single polypeptide chain of the variable domain that comprises the full antigen-binding capacity of the nanobody preferably has an amino acid sequence and structure that can be considered to be comprised of four framework regions or “FRs”, which are referred to in the art and herein as “Framework region 1” or “FR1”; a “Framework Region 2” or “FR2”; as “Framework Region 3” or “FR3”; and as “Framework Region 4” or “FR4”, respectively; which framework regions are interrupted by three complementarity determining regions or “CDRs”, which are referred to in the art as “Complementarity Determining Region 1” or “CDR1”; as “Complementarity Determining Region 2” or “CDR2”; and as “Complementarity Determining Region 3” or “CDR3”, respectively. These framework regions and complementarity determining regions are preferably operably linked in the order FR1-CDR1-FR2-CDR2-FR3-CDR3-FR4 (from amino terminus to carboxy terminus).

An example of peptide tags to which sdAbs were developed are the human beta-catenin derived peptides, such as the BC2T peptide PDRKAAVSHWQQ (SEQ ID NO: 1), representing a conserved epitope in human beta-catenin, and nanobodies thereto described in Traenkle et al., 2015, Molecular & Cellular Proteomics, 14(3), 707-723 and EP3377632. This peptide sequence formed the basis for the Spot-Tag@peptide tag, an inert 12 amino acid peptide-tag with the amino acid sequence PDRVRAVSHWSS (SEQ ID NO: 44), and Spot-Nanobodies that specifically bind to Spot-tagged proteins as offered by ChromoTek.

SdAbs recognizing a peptide tag of only 4 or 5 amino acids (AA), used as an affinity tag, are commercially available (e.g., the CaptureSelect® C-Tag matrix based on a 4AA peptide tag, E-P-E-A, and corresponding CaptureSelect® Biotin Anti-C-tag Conjugate consisting of a 13 kDa camelid antibody fragment (affinity ligand) with high affinity and selectivity for the 4-amino acid “C-tag” peptide tag E-P-E-A (SEQ ID NO: 45), offered by ThermoFisher). Another tag sequence and corresponding sdAb is the “ALFA-tag” as described by Gotzke et al., 2019, Nature Communications, 10:4403, https://doi.org/i0.1038/s41467-019-12301-7. The antigenic peptide amino acid sequence of the minimal ALFA-tag is SRLEEELRRRLTE (SEQ ID NO: 46). This sequence is capable of forming a stable α-helix. A sdAb specific for the ALFA-tag, NbALFA, was developed and binds to the peptide with low picomolar affinity. The ALFA-tag and variants thereof, as well as sdAbs are also mentioned in WO2020/053239 (Nanotag Biotechnologies).

Nanobodies generally have a low immunogenicity due to their small size and high sequence identity with human IGHV3 family gene products, meaning that they do hardly give rise to anti-drug antibodies (ADAs) (Ackaert C et al., 2021, Front. Immunol. 12:632687, doi: 10.3389/fimmu.2021.632687; Ren et al., supra; Joveevska et al., 2020, BioDrugs, 34, 11-26; Rossotti M et al., 2021, FEBS J., doi:10.1111/febs.15809).

SdAbs are known to have a relatively high affinity for their target antigenic peptide (low-nanomolar to picomolar range), which is preferred in many known applications where the binding partners should bind to each other even in very low concentrations (low K_{D}). In the present invention nanobodies, with a sub-nM K_{D} (picomolar) affinity can be used. The K_{D} for such antibodies is typically in the low picomolar (10-30 pM) range. The K_{D} value for the binding of the NbALFA for its ALFAtag, for example is approximately 26 pM. The affinity of the BC2 Nb for its BC2T peptide tag is 1.4 nM (1400 pM).

The use of high affinity sdAbs/antigenic peptide pairs results in efficient masking and demasking of masked antibodies or antigen binding fragments according to the invention. In a masked antibody or antigen binding fragment according to the invention, the masking moieties are brought in close proximity to each other because they are linked to the N-termini of a light and heavy chain pair, respectively, within a (masked) antibody. Due to their close proximity, the masking moieties of a mask may bind to each other even if they have a relatively low affinity towards each other. The high affinity between nanobodies and peptide tags, preferred for other applications, may thus not be a prerequisite for their use in masking moieties in masked antibodies, antigen binding fragments, or ADCs according to the invention.

In the context of the present invention, it may be an advantage to use sdAb with a relatively low affinity towards the antigenic peptide sequence specifically recognized. Using a sdAb with relatively low affinity for the antigenic peptide has the advantage that the ability of the masking moieties to form intermolecular bonds (i.e., bonds between masking moieties on the HC/LC of different antibodies or antigen binding fragments that may give rise to HMW complex formation) may be reduced. The percentage of HMW forming antibodies of the total amount of antibodies (HMW (%) in production is preferably below 10%.

At the same time their ability to form intramolecular bonds (i.e., a bond between masking moieties on a heavy and light chain pair within an antibody or antigen binding fragment to form a mask) is retained at lower affinity, due to the closer proximity of the two masking moieties, when conjugated to an HC and LC pair.

With a relatively low affinity is meant an affinity that is significantly (e.g., 2-100 and at least 2-10 times) lower than the high (single) nanomolar to picomolar range common for sdAb-epitope binding. The K_{D} of the binding between a Nb/peptide pair used in masking moieties in the present invention thus can be in the range from 10 pM (high affinity) to relatively low affinities where the K_{D} may be as high as 500 nM (0.01 nM to 500 nM).

Preferably, a sdAb and antigenic peptide are selected wherein the K_{D} value for the affinity between the sdAb and the antigenic peptide is in the 0.1-500 nM range, more preferably in the 1-100 nM range.

Within a panel of sdAbs raised against a certain epitope sequence, sdAbs can be selected for a lower affinity towards the antigenic peptide. In the alternative, certain sdAbs originally selected for their high affinity towards an antigenic peptide may be modified (e.g., by introducing certain point mutations in the binding region of the sdAb, to create variant sdAbs with a slightly altered amino acid sequence, reducing their affinity towards the peptide. Also, an antigenic peptide sequence, which is recognized with high affinity by a sdAb, can be altered to reduce the binding to the same sdAb.

Good results were obtained with masking moieties based on a beta-catenin based peptide (BC2T) and nanobody (BC2) thereto, as described in Traenkle et al., 2015, Molecular & Cellular Proteomics, 14(3), 707-723. The BC2T peptide described by Traenkle et al. has the amino acid sequence PDRKAAVSHWQQ (SEQ ID NO: 1), representing a conserved linear epitope corresponding to amino acid residues 16-27 of beta-catenin, while further variants, preserving the epitope, are described in EP3377632, where a peptide is disclosed with the following sequence: X₁X₂RX₄X₅AX₇SX₉WX₁₁X₁₂, wherein X₁ can be P or A, wherein X₂ can be D or a conservative substitution of D, wherein X₄ can be K, or a conservative substitution of K, or S, wherein X₅ can be A or R, or a conservative substitution of A or R, wherein X₇ can be V or a conservative substitution of V, wherein X₉ can be H or a conservative substitution of H, wherein X₁₁ and X₁₂ can independently of one another be Q or a conservative substitution of Q.

The term “conservative substitution” as used herein, means the substitution of one amino acid by another, wherein the replacement results in a silent alteration. This means that an amino acid residue can be substituted by another amino acid of a similar polarity which acts as a functional equivalent. Substitutes for an amino acid may be selected from other members of the class to which the amino acid belongs (i.e. a conservative substitution). For example, one polar amino acid can be substituted by another polar amino acid, one positively or negatively charged amino acid, respectively, can be substituted by another positively or negatively charged amino acid, respectively, et cetera. Classes of amino acids are for example, nonpolar (hydrophobic) amino acids including alanine, leucine, isoleucine, valine, proline, phenylalanine, tryptophan and methionine; polar neutral amino acids including glycine, serine, threonine, cysteine, tyrosine, asparagine, and glutamine; positively charged (basic) amino acids including arginine, lysine and histidine; negatively charged (acidic) amino acids including aspartic acid and glutamic acid.

Specific variant peptides displaying the fixed amino acids on positions 3(R), 6(A), 8(S) and 10(W), deemed essential for the epitope binding, are disclosed in EP3377632 as well, such as PVRSAALSQWSS (SEQ ID NO: 48), PDRVRAVSHWSS (SEQ ID NO: 49), and ADRVRAVSHWSS (SEQ ID NO: 50).

Traenkle et al. also describe nanobodies specifically binding to the BC2T peptide, defined by their CDR3 sequences. Out of their best performing nanobodies (BC1, BC2, BC6 BC9, and BC13), BC1 and BC2 bound to the recombinant beta-catenin protein with a K_{D} in the low nanomolar (˜1.9 nM and ˜3.1 nM) range, as measured with Surface Plamon Resonance Spectroscopy. BC13 showed a slightly lower affinity of ˜44 nM. For BC6 and BC9 detected affinities were in the low micromolar range. The CDR3 sequence for the BC2 nanobody is: ARGCKRGRYEYDFW (SEQ ID NO: 5).

The BC2T peptide and variants thereof, as well as the BC2-Nb (and variants thereof) specifically binding to said BC2T or variant peptide sequences, can be used in masking moieties to create masked antibodies, antigen binding fragments and/or ADCs according to the invention.

Whereas, for prior art uses of the peptide tag and sdAb sequences, pairs were selected for their high affinity binding, the very high affinity may not be required for the use in masking moieties in masked antibodies, antigen binding fragments or ADCs according to the invention. It was found that, by using an antigenic peptide/sdAb pair with relatively low affinity towards each other (in the 1-100 nM range) good results were obtained. Variants of the BC2 nanobody are described in Braun et al., 2016, Sci Rep 6, 19211, https://doi.org/10.1038/srep19211. Braun et al. mutated the CDR3 region of the BC2 nanobody by replacing Arg106 with either a serine (BC2-Nb_{R106S} or “RS” mutant (SEQ ID NO: 8) or a glutamate (BC2-Nb_{R106E} or “RE” mutant, (SEQ ID NO: 7)). In doing so the nanobody-peptide conformation of the BC2 nanobody (“headlock”) was disturbed, resulting in a 10-fold lower binding affinity (K_{D} values of ·11 nM; the RE mutant showed a K_{D} of 12 nM and the RS mutant showed a K_{D} of 9.7 nM) compared to the high-affinity BC2-Nb (K_{D}˜1.4 nM) as measured using surface plasmon resonance spectroscopy (SPR)-based affinity measurements. The K_{D} for the RA mutant is described in Ren et al., Journal of Chromatography A 1676 (2022) 463274. Ren et al. provide the same K_{D} for the wt BC2-Nb, 1.4 nM, as Braun et al., and compare it to the affinity for an R106Q mutant (K_{D}=282 nM), R106A mutant (K_{D}=360.5 nM) and an R106H mutant (K_{D}=335.5 nM).

Both the original BC2-Nb as well as the RS and RE mutants, and a mutant wherein the arginine was replaced by an alanine (BC2-Nb_{R106A} or “RA” mutant (SEQ ID NO: 6)) were tested in a masking moiety in a masked antibody according to the invention. The use of the mutant Nbs resulted in a significant lower (5-10 times lower) percentage of HMW complexes, for example, when used as masking moieties for anti-HER2 antibody trastuzumab, while the ability of the masking moieties to shield the binding site of the masked antibody until at least one of the linkers was cleaved was retained.

In the alternative, or in addition, to modifying the sdAb sequence to tweak affinity of high affinity peptide/sdAb pairs, the sequence of an antigenic peptide can be modified with the aim to reduce the affinity of the peptide-sdAb interaction. For example, in case of the BC2T peptide, the serine at position 8, which is fixed in the formula for the peptide sequence in EP3377632 may be replaced, for example by an alanine or threonine residue in peptides used in masking moieties of the invention. When the S(8) is replaced by X₈, this alters the general formula for the peptide to X₁X₂RX₄X₅AX₇X₈X₉WX₁₁X₁₂ (SEQ ID NO: 51), wherein X₁ can be P or A, wherein X₂ can be D or a conservative substitution of D, wherein X₄ can be K, or a conservative substitution of K, or S, wherein X₅ can be A or R, or a conservative substitution of A or R, wherein X₇ can be V or a conservative substitution of V, wherein X₈ can be S, A, V or T, wherein X₉ can be H or a conservative substitution of H, wherein X₁₁ and X₁₂ can be Q or a conservative substitution of Q. In the variant formula disclosed in EP3377632, the fixed “R” on position 3 can further ne replaced by a “K”, resulting in the following formula: X₁X₂X₃X₄X₅AX₇X₈X₉WX₁₁X₁₂ (SEQ ID NO: 95), wherein X₁, X₂, X₄X₅, X₇, X₈, X₉, X₁₁ and X₁₂ have the aforementioned meaning and X₃ can be R or K.

In a preferred peptide according to the invention X₈ is A or T, most preferably A, resulting, for example, in a peptide with the sequence PDRKAAVAHWQQ ((BC2T SA mutant, SEQ ID NO: 2) or PDRKAAVTHWQQ (BC2T ST mutant, SEQ ID NO: 3), preferably PDRKAAVAHWQQ.

Variant peptides with a substitution of S at the 8 position by A or T at the 8 position, such as BC2T ST mutant or BC2T SA mutant, can be used in a pair of masking moieties together with a masking moiety comprising the BC2-Nb or, preferably, with a masking moiety comprising a variant of said BC2-Nb moiety with a mutation in the CDR3 region, such as the BC2 RE, RS, or RA mutant. Good results were obtained with a pair of masking moieties comprising a masking moiety with the BC2T SA mutant peptide and a masking moiety with the BC2-Nb RS mutant nanobody.

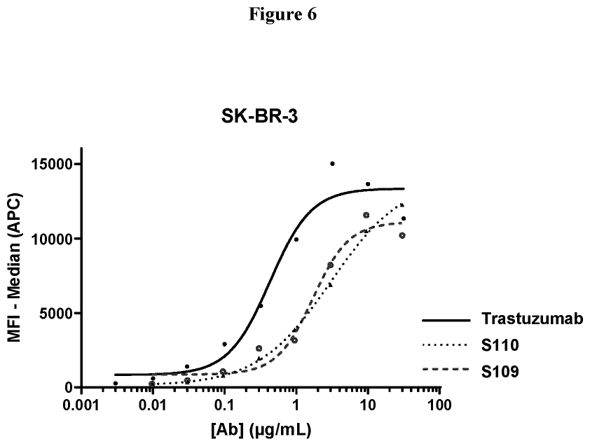

Also for another nanobody, the ALFA-tag, sequence variants were described in WO2020/053239 that differed in their affinity with the described sdAb. Peptides, based on different variant core structures had either a K_{D} for binding to the sdAb of about 1-5 nM, or, when the core structure was different, a K_{D} of about 10⁻⁵⁰ nM for binding to the same sdAb. An example of a modified ALFA tag Nb is the Nb^{PE} mutant, disclosed as SEQ ID NO:134 in WO2020/053239. In Gotzke et al., 2019, Nature Communications, 10:4403, https://doi.org/10.1038/s41467-019-12301-7, it is described that this mutant was designed for protein elution. Where the original ALFA tag Nb binds to the tag peptide with high picomolar affinity (K_{d} is 26 pM) the Nb^{PE} mutant (“PE” for Peptide-Elution) binds with lower affinity (K^{d} is 11 nM), as reflected in supplementary FIG. 6 of Gotzke et al. Using the Nb^{PE} mutant and the ALFA tag peptide in masking moieties, attached to an antibody with protease cleavable linkers, antibodies could efficiently be masked and demasked.

In the alternative to, or in addition to, using low-affinity sdAbs/antigenic peptide pairs as masks to prevent HMW complex formation, prior art methods to remove or reduce the formation of HMW complexes in protein production, purification and storage can be used as well.

### Linkers

At least one of the masking moieties (either the antigenic peptide or the sdAb or both) may be linked to the antibody by a cleavable linker, preferably a conditionally cleavable peptidic linker which will be cleaved only at the target site (the site where the antibody should bind to its target antigen and the mask should be removed, e.g., in or at a tumor site). Preferably, the cleavable peptidic linker is a protease cleavable peptidic linker. Preferably, both masking moieties are linked to the antibody by a cleavable linker.

The binding capacity of the antibody or antigen binding fragment may already be sufficiently restored for the antibody to have its desired therapeutic effect, when only one linker is cleaved. Preferably, all linkers attaching the masking moieties to an antibody or antigen binding fragment are cleaved and the mask is completely detached from the antibody after cleavage.

Peptide linkers, used to join the different parts of a fusion proteins are known in the art. Such linkers may be flexible linkers, rigid linkers, cleavable linkers or a combination (Chen et al., 2013, Adv Drug Deliv Rev., 65(10), 1357-1369). A cleavable linker for use in masked antibodies or antigen binding fragments according to the invention, preferably, is a peptide linker comprising one or more cleavage site(s) recognized by one or more proteases.

When the masked antibodies are used in cancer therapy, the cleavable peptidic linker should preferably comprise at least one cleavage site recognized by at least one tumor specific protease. Examples of such proteases known in the art are matriptase (MT-SP1, a tumor-associated type II transmembrane serine protease), Cathepsin B, urokinase-type plasminogen activator (uPA) and/or metalloproteinases (MMPs) such as MMP2, MMP9, and MMP14, which are overexpressed in almost all types of cancer (Li Yanan et al., 2021, Acta Pharmaceutica Sinica B, 11(8), 2220-2242). Linkers comprising tumor-specific protease cleavage sites are known in the art, also in the context of antibody masking technologies for example WO2018/107125 (Seattle Genetics), WO2009/025846, WO2010/081173 and WO2016/118629 (Cytomx Therapeutics LLC) and Choi et al., 2012, Theranostics, 2(2), 156-178.

A cleavage site is the (minimal) amino acid sequence recognized by a protease, and the actual cleavage will be within this site. An example of cleavage sites known in the art is the MMP cleavage site PLGLAG, SEQ ID NO: 13, which is cleavable by MMP2 as well as MMP9 (Jiang et al., 2004, PNAS, 101(51), 17867-17872; Trang et al., 2019, Nature biotechnology, 37(7), 761-765.). Other known cleavage sites are for example the cleavage site LSGRSDNH (SEQ ID NO: 14) (EP2385955, Cytomx Therapeutics LLC), recognized by urokinase-type plasminogen activator (uPA) as well as a cleavage site for MMP14 with the sequence ISSGLL (SEQ ID NO: 15) (WO2016/118629, Cytomx Therapeutics LLC). Other cleavage sites, such as RQARVVNG (SEQ ID NO: 53), and PMAKK (SEQ ID NO: 54), recognized by matriptase, are described in WO2017/162587 (Hoffmann-La Roche AG). Yet other cleavage sites, PLGVR (SEQ ID NO: 70) and IPVSLR (SEQ ID NO: 52), or IPVSLRSG (SEQ ID 75) are MMP2 cleavable sites mentioned in, for example, WO2018107125 (Seattle Genetics Inc.) and in Lin et al. supra. Preferred cleavage sites for use in masked antibodies or antigen binding fragments according to the invention are PLGLAG, IPVSLR(SG), LSGRSDNH, and/or PMAKK.

Preferably, both masking moieties are linked with linkers containing at least one protease cleavage site. Linkers that are cleaved by multiple proteases may be used to increase activation at the target site, e.g., a tumor, where at least one masking moiety should be cleaved off to restore binding of the demasked antibody. Such linkers may comprise cleavage sites recognised by multiple proteases and/or different cleavage sites each recognised by a different protease. An example of cleavable linker sequences comprising multiple cleavage sites recognized by different proteases are, for example, described as “2001” in WO2016/118629 (Cytomx Therapeutics LLC). The “2001” sequence has the AA sequence ISSGLLSGRSDNH (SEQ ID NO: 16) and combines the ISSGLL cleavage site for MMP14 with the LSGRSDNH cleavage site recognized by matriptase as well as uPA. Cleavage sites can be combined in different orders (WO2016/118629).

Both linkers of a pair of masking moieties forming a mask may contain the same (identical) or (a) different (non-identical) cleavage site(s). Preferred combinations of linkers used to conjugate a pair of masking moieties forming a mask to a HC/LC pair of an antibody or antigen binding fragment are combinations of linkers where both linkers comprise cleavage sites selected from the group PLGLAG, LSGRSDNH, IPVSLR(SG) and PMAKK.

Preferred combinations of linkers include a combination of a linker comprising a cleavage site recognized by matriptase for conjugating the masking moiety with the SdAb to the antibody, preferably to the LC VR, combined with a linker comprising a cleavage site recognized by a metalloprotease, for example, MMP2, conjugating the masking moiety with the antigenic peptide to the antibody or antigen binding fragment, preferably to the HC VR of the same HC/LC binding pair. The masking moiety conjugated to the LCVR of at least one HC/LC chain pair preferably comprises the BC2-Nb RS mutant nanobody, and is conjugated to the LCVR by a linker comprising a cleavage site recognized by a metalloprotease, preferably PLGLAG, while the masking moiety conjugated to the HCVR of the HC/LC chain pair comprises the BC2T SA mutant peptide, and is conjugated to the HC by a linker comprising a cleavage site recognized by matriptase, preferably LSGRSDNH. In the alternative, the cleavage site recognized by a metalloprotease may be ISSGLL or IPVSLR(SG) while the alternative for the matriptase cleavage site may be PMAKK.

In addition to the actual protease cleavage site(s), linkers used to link masking moieties to the HC and/or LC of a masked antibody or antigen binding fragment according to the invention may include additional spacer sequences such as serine and/or glycine rich stretches, mainly to increase flexibility of the linker, for example to provide for sufficient movement for the masking moieties to adopt the appropriate orientation to bind to each other and to optimize the degree of shielding of the actual mask. Linkers may be optimized for use with a particular antibody by varying the spacer sequences (e.g., in length) and the location of the cleavage site within the linker.

Commonly used flexible linkers are linkers containing glycine rich regions, such as for example -GGS- or -(GGGGS)n-, (Chen et al., supra). In a linker for use in masked antibodies according to the invention, such (a) glycine (/serine) rich spacer sequence(s) may be joined to the actual protease cleavage site, either at one or at both ends of the cleavage site sequence(s) within a cleavable linker. Separate cleavage recognition sequences can be directly joined, or they can be separated (and/or flanked) by G/S rich sequences, and the separate cleavage site can be placed in different order (for example ISSGLLSSGGSGGSLSGRSDNH (SEQ ID NO: 17), or LSGRSDNHGGSGGSISSGLLSS (SEQ ID NO: 18), mentioned in WO2016/118629).

Preferred linkers therefore contain one or more proteolytic cleavage sites flanked by one or more spacer sequences. Most preferred are linkers where there is no, or only a short flanking sequence between the N-terminus of the HCVR or LCVR of the antibody or antigen binding fragment sequence and the (first) cleavage site. Depending on where the linker is cleaved, of course a part of the linker(s) sequence(s) may remain attached to the N-terminus of the HC and/or LC of a demasked antibody after cleavage of the linker. When a cleavage site is close to the N-terminus of the HC and/or LC sequence, no or only a few amino acids will remain attached to the HC and/or LC after the linker has been cleaved. If a spacer sequence is present between the N-terminus of the HC and/or LC and a cleavage site, such a sequence is preferably short, preferably 0-10, or 1-5 amino acids in length, for example 3 amino acids in length, such as amino acid sequence -GGS-. An additional spacer sequence may be included at the N′ terminus of the cleavage site sequence(s), for example a -GGS- or -GGGGS-spacer sequence. A resulting linker may for example have the sequence -GGGGSPLGLAGGGS-(SEQ ID NO: 11), wherein the MMP cleavage site is underlined. Resulting linkers may be of any suitable length, depending on the length and number of the cleavage recognition sites used and the length of the flexible linkers put in front and/or behind said cleavage site sequence(s). Suitable linkers may generally comprise from 2-30, preferably from 5-25 amino acids.

Although antibodies or antigen binding fragments have the same overall conformation, each respective antibody or antigen binding fragment has a different antigen binding region. Antigen binding regions may differ in sequence, and therefore in charge distribution and 3D conformation. Although the present invention provides an elegant and uniform method for masking antibodies or antigen binding fragments, whereby the same masking moieties can in principle be used on every antibody or antigen binding fragment with good results, tweaking the linker length may result in, for example, a higher yield during production. To that effect, a set of linkers differing in length can be made and tested in different combinations as pairs to link masking moieties to the LC and HC. When different combinations are tested, the best performing linker set may be chosen. “Best performing” can be defined on the basis of a variety of measured parameters. For example, a linker pair can be chosen for which expression levels are high and HMW (%) in production is very low when compared to other linker pairs tested, while masking and demasking efficiency is still retained.

For example, linkers can be chosen from the set reflected in Table 18 (as tested in Example 10 for one particular antibody, tisotumab). In the set reflected in Table 18, linkers L14-L25 all contain the protease cleavage site LSGRSDNH, but differ in length, while linkers L26, L27, L9, L29, L7, and L30 all contain the PLGLAG protease cleavage site and likewise differ in length. This set of linkers encompasses 324 possible combinations and allows for testing of combinations of linkers with the same or different cleavage sites for both masking moieties, while the length of the linkers may also be the same or different. In Example 10, where as an example all 324 linker combinations were tested, it is exemplified that the majority could be produced at expression levels above 100 μg/mL. HMW (%) was below 10% in the majority of the cases, while for 198 linker combinations HMW (%) was below 5%, and for 39 even below 3%.

A preferred subset of linker combinations to create these 324 antibodies is the subset of linker combinations used for creating the ADCs 1-12 reflected in table 22 in Example 11.

This exercise may be repeated with all or only a subset of possible combinations chosen from Table 18 for any antibody or antigen binding fragment to be masked. The present invention encompasses masked antibodies or antigen binding fragments thereof, where masking moieties are conjugated to at least one HC/LC pair of the antibody or antigen binding fragment, using any suitable linker combination chosen from the linkers reflected in Table 18.

Spacer sequences, such as QGQSGQG (Qg) (SEQ ID NO: 9) or QVQLVES (Qv) (SEQ ID NO: 10) can also be included in the expressed protein sequences, for example, at the N-terminus of the antigenic peptide of a masking moiety (whereas the linker is attached to the C-terminus of the peptide).

In N—C order, when a peptide is attached to the HC, the construct may thus have the following conformation: Qg-antigenic peptide-linker-HC.

The Qg spacer sequence is known in the art (e.g., Cytomx WO2016/179285).

The Qv sequence is based on a conserved sequence found in a framework 1 region of the heavy chain variable domain (VH) sequence in many human antibodies. By introducing a spacer sequence like the Qg or Qv sequence in front of (at the N-terminus of) the amino acid sequence of the antigenic peptide, production titers of masked antibodies according to the invention could be raised up to 10-fold, irrespective of whether the masking moiety with the antigenic peptide was linked to the N-terminus of the HC or LC. Good results were obtained with masked antibodies where the Qg or Qv sequence was placed in front of the BC2T peptide sequence (variant).

In masked antibodies according to the invention, at least one of the two masking moieties may be linked to the antibody or antigen binding fragment by a cleavable linker. Linking only one masking moiety with a cleavable linker may suffice when the binding capacity of the masked antibody or antigen binding fragment can already sufficiently be restored by cleaving the bond between one masking moiety and the antibody or antigen binding fragment. When only one of the masking moieties is linked by a cleavable linker to either the HC or the LC of the antibody or antigen binding fragment, and this linker is cleaved, the mask will be removed from the antigen binding site of the antibody or antigen binding fragment (its binding site will be “demasked”), despite the fact that a masking moiety may still be attached to the antibody or antigen binding fragment. When only one masking moiety is attached by a cleavable linker, this masking moiety preferably is the masking moiety comprising the sdAb.

Because of the small size of a mask as used with the present invention, the actual binding site of the antibody or antigen binding fragment is available for binding to its target antigen and the masking moieties no longer prevent the antibody or antigen binding fragment from binding to its target, even when only one linker is cleaved. When one of the masking moieties, so either the sdAb or the antigenic peptide is bound by a cleavable (peptide) linker, the other masking moiety may also be linked by a peptidic linker, or, in the alternative, may be conjugated to the antibody or antigen binding fragment. For example, a fusion protein comprising the antibody or antigen binding fragment fused to a sdAb can be produced to which the antigenic peptide, serving as the second masking moiety, can be conjugated. In the alternative, both the sdAb as well as the antigenic peptide are included in the recombinantly produced (fusion) protein sequences, and either one or both are linked via a (cleavable) peptide linker which is part of the fusion protein(s) expressed.

In a masked antibody or antigen binding fragment according to the invention, the antigenic peptide and the sdAb may be linked to the N-termini of at least one of the light and heavy chain pairs of the antibody or antigen binding fragment. In a bivalent antibody or antibody fragment with two HC/LC pairs (e.g. a F(ab′)₂ fragment), preferably all (both) HC/LC pairs are provided with a mask.

In a masked antibody or antigen binding fragment according to the invention, the masking moiety comprising an antigenic peptide may be connected to the N-terminus of the LC, while the sdAb may be connected to the N-terminus of the HC, or vice versa.

From a production perspective, it may have an advantage to use a masked antibody wherein the masking moiety with the sdAb is connected to the N-terminus of the LC while the antigenic peptide is connected to the N-terminus of the HC, is preferred. In experiments performed, these antibodies were produced with higher titers (and lower HMW (%)).

The masked antibodies or antigen binding fragments according to the invention are especially suitable for use in antibody-drug conjugates, because the mask does not interfere with conjugation of the masked antibody or antigen binding fragment to a linker-drug. Targeting antibodies or antigen binding fragments, that may be used in antibody-drug conjugates for use in cancer therapy, may be a tumor targeting antibody or antigen binding fragment, selectively binding to a tumor-specific or tumor-associated antigen. Tumor-specific antigens only occur on tumor cells, while tumor associated antigens are antigens that are expressed at higher levels (e.g., overexpressed) in cancer cells, when compared to normal (healthy) cells.

The antigen target (also termed “target protein” herein) to which a masked antibody or antigen binding fragment according to the invention binds may be selected from the group consisting of: annexin A1, B7H3, B7H4, BCMA, CA6, CA9, CA15-3, CA19-9, CA27-29, CA125, CA242 (cancer antigen 242), CAIX, CCR2, CCR5, CD2, CD3, CD19, CD20, CD22, CD24, CD28, CD30 (tumor necrosis factor 8), CD33, CD37, CD38 (cyclic ADP ribose hydrolase), CD40, CD44, CD47 (integrin associated protein), CD56 (neural cell adhesion molecule), CD70, CD71, CD73, CD74, CD79, CD115 (colony stimulating factor 1 receptor), CD123 (interleukin-3 receptor), CD138 (Syndecan 1), CD203c (ENPP3), CD303, CD333, CDCP1, CEA, CEACAM, Claudin 4, Claudin 7, CLCA-1 (C-type lectin-like molecule-1), CLL 1, c-MET (hepatocyte growth factor receptor), Cripto, CTLA4, DLL3, EGFL, EGFR, EPCAM, EphA2, EphB3, ETBR (endothelin type B receptor), FAP, FcRL5 (Fc receptor-like protein 5, CD307), FGFR3, FOLR1 (folate receptor alpha), Frbeta, GCC (guanylyl cyclase C), GD2, GITR, GLOBO H, GPA33, GPC3, GPNMB, HER2, p95HER2, HER3, HMW-MAA (high molecular weight melanoma-associated antigen), integrin α (e.g., αvβ3 and αvβ5), IGF1R, TM4SF1 (L6), Lewis A like carbohydrate, Lewis X, Lewis Y (CD174), LGR5, LIV1, mesothelin (MSLN), MN (CA9), MUC1, MUC16, NaPi2b, Nectin-4, Notch3, PD-1, PD-L1, PSMA, PTK7, SLC44A4, STEAP-1, SIRP-alpha, 5T4 (or TPBG, trophoblast glycoprotein), TF (tissue factor, thromboplastin, CD142), TF-Ag, Tag72, TNFalpha, TNFR, TROP2 (tumor-associated calcium signal transducer 2), uPAR, VEGFR and VLA.

The masked antibody or antigen binding fragment thereof, if applicable, may comprise (1) a constant region that is engineered, i.e., one or more mutations may have been introduced to e.g., increase half-life, provide a site of attachment for the linker-drug and/or increase or decrease effector function; and/or (2) a variable region that is engineered, i.e., one or more mutations may have been introduced to e.g., provide a site of attachment for the linker-drug. Antibodies or antigen binding fragments thereof may be produced recombinantly, synthetically, or by other known suitable methods. Mutations that may decrease Fc mediated effector function of antibodies or antigen binding fragments are, for example, mutations such as those described in Leabman et al., Mabs, 2013, 5(6):896-903.

### ADCs

The present invention further provides antibody-drug conjugates based on masked antibodies or antigen binding fragments according to the invention. An ADC according to the invention comprises a masked antibody or antigen binding fragment according to the invention to which one or more linker-drug compounds have been conjugated. Methods to produce ADCs are known in the art. Masked antibodies or antigen binding fragments according to the invention can be used in the production of ADCs according to conventional methods.

To synthesize a conjugate according to the invention, one or more linker-drug compound(s) may be conjugated to a masked antibody or antigen binding fragment according to the invention. The “drug” in an ADC is a pharmacologically active moiety; for example a cytotoxic moiety or an immunomodulatory moiety or any moiety that exerts a direct or indirect desired pharmacological effect on, or via, a cell to which the antibody or antigen binding fragment of the ADC can bind.

Suitable linker-drugs for the use in ADCs are known in the art. For example, ADCs based on duocarmycin based linker-drugs (such as trastuzumab based SYD985) are disclosed in WO2011/133039 and Elgersma et al., 2015, Molecular Pharmaceutics, 12, 1813-1835. Elgersma et al. disclose the synthesis and use of a seco-DUBA based linker-drug (linker-drug 2 or LD2 (SYD980).

The linker-drug compound may be conjugated via a reactive native amino acid residue present in the suitable polypeptide, e.g., a lysine or a cysteine, or via an N-terminus or C-terminus. Alternatively, a reactive amino acid residue, natural or non-natural, may be genetically engineered into the suitable polypeptide, or a reactive group may be introduced via post-translational modification.

ADCs according to the invention may be produced by conjugating a linker-drug compound to a masked antibody or antigen binding fragment thereof through e.g., the lysine ε-amino groups of the antibody or antigen binding fragment, preferably using an intermediate comprising an amine-reactive group such as an activated ester. Such methods are known for producing conventional ADCs. Alternatively, ADCs can be produced by conjugating the linker through the free thiols of the side chains of cysteines generated through reduction of interchain disulfide bonds, using methods and conditions known in the art, see e.g., Doronina et al, 2006, Bioconjugate Chem. 17, 114-124. The manufacturing process involves partial reduction of the solvent-exposed interchain disulfides followed by modification of the resulting thiols with Michael acceptor-containing linkers such as maleimide-containing linkers, alfa-haloacetic amides or esters. The cysteine attachment strategy results in maximally two linker containing linker-drugs per reduced disulfide.

Preferred antibodies according to the invention are of the human IgG type. Most human IgG molecules have four solvent-exposed disulfide bonds, which equates to a range of integers of from zero to eight linked linking moieties per antibody. The exact number of linker-drug molecules per antibody (or antigen binding fragment) is determined by the extent of disulfide reduction and the number of molar equivalents of linker containing linker-drugs in the ensuing conjugation reaction. Full reduction of all four disulfide bonds gives a homogeneous construct with eight linker moieties per antibody, while a partial reduction typically results in a heterogeneous mixture with zero, two, four, six, or eight linking moieties per antibody.

In a preferred embodiment, the present invention relates to an ADC, wherein the linker-drug compound is conjugated to a masked antibody or antigen binding fragment thereof through a cysteine residue of the antibody or the antigen binding fragment.

Because antibodies contain many lysine residues and cysteine disulfide bonds, conventional conjugation typically produces heterogeneous mixtures that present challenges with respect to analytical characterization and manufacturing. Furthermore, the individual constituents of these mixtures exhibit different physicochemical properties and pharmacology with respect to their pharmacokinetic, efficacy, and safety profiles, hindering a rational approach to optimizing this modality.

To improve conjugate homogeneity, masked antibodies or antigen binding fragments used to create masked ADCs according to the invention may be modified to allow for site-specific conjugation of the linker-drug molecules. Methods for site-specific drug conjugation to antibodies (or antigen binding fragments) are comprehensively reviewed by C. R. Behrens and B. Liu, mAbs, 2014, 6 (1), 1-8, WO2005/084390, and WO2006/034488. Site-specific ADCs are preferably produced by conjugating the linker-drug compound to the antibody or antigen binding fragment thereof through the side chains of engineered cysteine residues in suitable positions of the mutated antibody or antigen binding fragment. Engineered cysteines are usually capped by other thiols, such as cysteine or glutathione, to form disulfides. These capped residues need to be uncapped before linker-drug attachment can occur. Linker-drug attachment to the engineered residues is either achieved (1) by reducing both the native interchain and mutant disulfides, then re-oxidizing the native interchain cysteines using a mild oxidant such as CuSO₄ or dehydroascorbic acid, followed by standard conjugation of the uncapped engineered cysteine with a linker-drug, or (2) by using mild reducing agents which reduce mutant disulfides at a higher rate than the interchain disulfide bonds, followed by standard conjugation of the uncapped engineered cysteine with a linker-drug. Suitable methods for site-specifically conjugating linker-drugs can for example be found in WO2015/177360 which describes the process of reduction and re-oxidation, WO2017/137628 which describes a method using mild reducing agents and WO2018/215427 which describes a method for conjugating both the reduced interchain cysteines and the uncapped engineered cysteines, as well as in Coumans et al., 2020, Bioconjugate Chem 31(9), 2136-2146, https://doi.org/10.1021/acs.bioconjchem.0c00337.

### Pharmaceutical Compositions

In a further aspect, the invention provides a composition comprising a masked antibody, a masked antigen binding fragment, or a masked ADC according to the invention, preferably wherein the composition is a pharmaceutical composition, more preferably further comprising one or more pharmaceutically acceptable excipient(s). Such composition is referred to hereinafter as a composition according to the invention. The composition may for example be a liquid formulation, a lyophilized formulation, or in the form of e.g., capsules or tablets.

Typically, pharmaceutical compositions according to the invention take the form of lyophilized cakes (lyophilized powders), which require (aqueous) dissolution (i.e., reconstitution) before intravenous infusion, or frozen (aqueous) solutions, which require thawing before use. Accordingly, in preferred embodiments, the invention provides a lyophilized composition comprising a masked antibody, a masked antigen binding fragment, or a masked ADC according to the invention, preferably wherein the composition is a pharmaceutical composition, more preferably further comprising one or more pharmaceutically acceptable excipient(s). In further preferred embodiments, the invention provides a frozen composition comprising water and a masked antibody, a masked antigen binding fragment, or a masked ADC according to the invention, preferably wherein the composition is a pharmaceutical composition, more preferably further comprising one or more pharmaceutically acceptable excipient(s). In this context, the frozen solution is preferably at atmospheric pressure, and the frozen solution was preferably obtained by freezing a liquid composition according to the invention at temperatures below 0° C. Suitable pharmaceutically acceptable excipients for inclusion into the pharmaceutical composition (before freeze-drying) in accordance with the present invention include buffer solutions (e.g., citrate, amino acids such as histidine, or succinate containing salts in water), lyoprotectants (e.g., sucrose, trehalose), tonicity modifiers (e.g., chloride salts, such as sodium chloride), surfactants (e.g., polysorbate), and bulking agents (e.g., mannitol, glycine). Excipients used for freeze-dried protein formulations are selected for their ability to prevent protein denaturation during the freeze-drying process as well as during storage.

### Medical Uses

In a further aspect, the invention provides a masked antibody or antigen binding fragment thereof or masked ADC according to the invention, or a composition according to the invention, for use as a medicament, preferably for the treatment of cancer, autoimmune or infectious diseases.

A masked antibody, a masked antigen binding fragment, a masked ADC or a composition according to the invention are collectively referred to hereinafter as products for use according to the invention. In one embodiment, the products for use according to the invention are for use in the treatment of a solid tumor or hematological malignancy. In a second embodiment, the products for use according to the invention are for use in the treatment of an autoimmune disease. In a third embodiment, the products for use according to the invention are for use in the treatment of an infectious disease, such as a bacterial, viral, parasitic or other infection.

A cancer in the context of the present invention, preferably is a tumor expressing the antigen to which the products for use according to the invention are directed. Such tumor may be a solid tumor or hematological malignancy. Examples of tumors or hematological malignancies that may be treated with products for use according to the invention as defined above may include, but are not limited to, breast cancer; brain cancer (e.g., glioblastoma); head and neck cancer; thyroid cancer; parotic gland cancer; adrenal cancer (e.g., neuroblastoma, paraganglioma, or pheochromocytoma); bone cancer (e.g., osteosarcoma); soft tissue sarcoma (STS); ocular cancer (e.g., uveal melanoma); esophageal cancer; gastric cancer; small intestine cancer; colorectal cancer; urothelial cell cancer (e.g., bladder, penile, ureter, or renal cancer); ovarian cancer; uterine cancer; vaginal, vulvar and cervical cancer; lung cancer (especially non-small cell lung cancer (NSCLC) and small-cell lung cancer (SCLC)); melanoma; mesothelioma (especially malignant pleural and abdominal mesothelioma); liver cancer (e.g., hepatocellular carcinoma); pancreatic cancer; skin cancer (e.g., basalioma, squamous cell carcinoma, or dermatofibrosarcoma protuberans); testicular cancer; prostate cancer; acute myeloid leukemia (AML); chronic myeloid leukemia (CML); chronic lymphatic leukemia (CLL); acute lymphoblastic leukemia (ALL); myelodysplastic syndrome (MDS); blastic plasmacytoid dendritic cell neoplasia (BPDCN); Hodgkin's lymphoma; non-Hodgkin's lymphoma (NHL) (including follicular lymphoma (FL), CNS lymphoma, and diffuse large B-cell lymphoma (DLBCL)); light chain amyloidosis; plasma cell leukemia; and multiple myeloma (MM).

An autoimmune disease in the context of the present invention, preferably is an autoimmune disease associated with the antigen to which the products for use according to the invention are directed. An autoimmune disease represents a condition arising from an abnormal immune response to normal body cells and tissues. There is a wide variety of at least 80 types of autoimmune diseases. Some diseases are organ specific and are restricted to affecting certain tissues, while others resemble systemic inflammatory diseases that impact many tissues throughout the body. The appearance and severity of these signs and symptoms depend on the location and type of inflammatory response that occurs and may fluctuate over time. Examples of autoimmune diseases that may be treated with products for use according to the invention as defined above may include, but are not limited to, rheumatoid arthritis; juvenile dermatomyositis; psoriasis; psoriatic arthritis; lupus; sarcoidosis; Crohn's disease; eczema; nephritis; uveitis; polymyositis; neuritis including Guillain-Barre syndrome; encephalitis; arachnoiditis; systemic sclerosis; autoimmune mediated musculoskeletal and connective tissue diseases; neuromuscular degenerative diseases including Alzheimer's disease, multiple sclerosis (MS), amyotrophic lateral sclerosis (ALS), neuromyelitis optica, and large, middle size, small vessel Kawasaki and Henoch Schonlein vasculitis; cold and warm agglutinin disease; autoimmune hemolytic anemia; type 1 diabetes mellitus; Hashimoto's thyroiditis; Graves' disease; Graves' ophthalmopathy; adrenalitis; hypophysitis; pemphigus vulgaris; Addison's disease; ankyloses spondylitis; Behcet's syndrome; celiac disease; Goodpasture's syndrome; myasthenia gravis; sarcoidosis; scleroderma; primary sclerosing cholangitis, epidermolysis bullosa acquisita, and bullous pemphigoid.

An infectious disease in the context of the present invention, preferably is an infectious disease associated with the antigen to which the products for use according to the invention are directed. Such infectious disease may be a bacterial, viral, parasitic or other infection. Examples of infectious diseases that may be treated with products for use according to the invention as defined above may include, but are not limited to, malaria; toxoplasmosis; pneumocystis jirovecii melioidosis; shigellosis; listeria; cyclospora; mycobacterium leprae; tuberculosis; and infectious prophylaxis in immune compromised individuals, such as in HIV-positive individuals, individuals on immunosuppressive treatment, or individuals with inborn errors such as cystic fibrosis or benign proliferative diseases (e.g., Mola hydatidosa or endometriosis).

Products for use according to the invention as described herein can be for the use in the manufacture of a medicament as described herein. Products for use according to the invention as described herein are preferably for methods of treatment, wherein the products for use are administered to a subject, preferably to a subject in need thereof, in a therapeutically effective amount. Thus, alternatively, or in combination with any of the other embodiments, in an embodiment, the present invention relates to a use of products for use according to the invention for the manufacture of a medicament for the treatment of cancer, autoimmune or infectious diseases, in particular for the treatment of cancer. For illustrative, non-limitative, cancers or other diseases to be treated according to the invention: see hereinabove.

Alternatively, or in combination with any of the other embodiments, in an embodiment, the present invention relates to a method for treating cancer, autoimmune or infectious diseases, in particular cancer, which method comprises administering to a subject in need of said treatment a therapeutically effective amount of a product for use according to the invention. For illustrative, non-limitative, cancers or other diseases to be treated according to the invention: see hereinabove.

Products for use according to the invention are for administration to a subject. Products for use according to the invention can be used in the methods of treatment described hereinabove by administration of an effective amount of the composition to a subject in need thereof. The term “subject” as used herein refers to all animals classified as mammals and includes, but is not restricted to, primates and humans. The subject is preferably a human. The expression “therapeutically effective amount” means an amount sufficient to effect a desired response, or to ameliorate a symptom or sign. A therapeutically effective amount for a particular subject may vary depending on factors such as the condition being treated, the overall health of the subject, the method, route, and dose of administration and the severity of side effects.

### Combined Use

In further embodiments, the invention provides the product for use according to the invention, wherein the use is combined with one or more other therapeutic agents. Products for use according to the invention may be used concomitantly or sequentially with the one or more other therapeutic agents. Suitable chemotherapeutic agents include alkylating agents, such as nitrogen mustards, hydroxyurea, nitrosoureas, tetrazines (e.g., temozolomide) and aziridines (e.g., mitomycin); drugs interfering with the DNA damage response, such as PARP inhibitors, ATR and ATM inhibitors, CHK1 and CHK2 inhibitors, DNA-PK inhibitors, and WEE1 inhibitors; anti-metabolites, such as antifolates (e.g., pemetrexed), fluoropyrimidines (e.g, gemcitabine), deoxynucleoside analogues and thiopurines; anti-microtubule agents, such as vinca alkaloids and taxanes; topoisomerase I and II inhibitors; cytotoxic antibiotics, such as anthracyclines and bleomycins; hypomethylating agents such as decitabine and azacitidine; histone deacetylase inhibitors; all-trans retinoic acid; and arsenic trioxide. Suitable radiation therapeutics include radio-isotopes, such as ¹³¹I-metaiodobenzylguanidine (MIBG), ³²P as sodium phosphate, ²²³Ra chloride, ⁸⁹Sr chloride and ¹⁵³Sm diamine tetramethylene phosphonate (EDTMP). Suitable agents to be used as hormonal therapeutics include inhibitors of hormone synthesis, such as aromatase inhibitors and GnRH analogues; hormone receptor antagonists, such as selective estrogen receptor modulators (e.g., tamoxifen and fulvestrant) and antiandrogens, such as bicalutamide, enzalutamide and flutamide; CYP17A1 inhibitors, such as abiraterone; and somatostatin analogs.

Targeted therapeutics are therapeutics that interfere with specific proteins involved in tumorigenesis and proliferation and may be small-molecule drugs; proteins, such as therapeutic antibodies; peptides and peptide derivatives; or protein-small molecule hybrids, such as ADCs. Examples of targeted small molecule drugs include TLR ligands, mTor inhibitors, such as everolimus, temsirolimus and rapamycin; kinase inhibitors, such as imatinib, dasatinib and nilotinib; VEGF inhibitors, such as sorafenib and regorafenib; EGFR/HER2 inhibitors, such as gefitinib, lapatinib, and erlotinib; and CDK4/6 inhibitors, such as palbociclib, ribociclib and abemaciclib. Examples of peptide or peptide derivative targeted therapeutics include proteasome inhibitors, such as bortezomib and carfilzomib.

Suitable anti-inflammatory drugs include D-penicillamine, azathioprine and 6-mercaptopurine, cyclosporine, anti-TNF biologicals (e.g., infliximab, etanercept, adalimumab, golimumab, certolizumab, or certolizumab pegol), lenflunomide, abatacept, tocilizumab, anakinra, ustekinumab, rituximab, daratumumab, ofatumumab, obinutuzumab, secukinumab, apremilast, acitretin, and JAK inhibitors (e.g., tofacitinib, baricitinib, or upadacitinib).

Immunotherapeutic agents include agents that induce, enhance or suppress an immune response, such as cytokines (IL-2 and IFN-α); immuno modulatory imide drugs, e.g., thalidomide, lenalidomide, pomalidomide, or imiquimod; therapeutic cancer vaccines, e.g., talimogene laherparepvec; cell based immunotherapeutic agents, e.g., dendritic cell vaccines, adoptive T-cells, or chimeric antigen receptor-modified T-cells; and therapeutic antibodies that can trigger antibody-dependent cell-mediated cytotoxicity (ADCC), antibody-dependent cellular phagocytosis (ADCP) or complement-dependent cytotoxicity (CDC) via their Fc region when binding to membrane bound ligands on a cell.

In the context of the invention, treatment is preferably preventing, reverting, curing, ameliorating, and/or delaying the cancer, autoimmune or infectious disease. This may mean that the severity of at least one symptom of the cancer, autoimmune or infectious disease has been reduced, and/or at least a parameter associated with the cancer, autoimmune or infectious disease has been improved.

In the context of the invention, a subject may survive and/or may be considered as being disease free. Alternatively, the disease or condition may have been stopped or delayed. In the context of the invention, an improvement of quality of life and observed pain relief may mean that a subject may need less pain relief drugs than at the onset of the treatment. “Less” in this context may mean 5% less, 10% less, 20% less, 30% less, 40% less, 50% less, 60% less, 70% less, 80% less, 90% less. A subject may no longer need any pain relief drug. This improvement of quality of life and observed pain relief may be seen, detected or assessed after at least one week, two weeks, three weeks, four weeks, one month, two months, three months, four months, five months, six months or more of treatment in a subject and compared to the quality of life and observed pain relief at the onset of the treatment of said subject.

### Nucleic Acid Construct

In a further aspect the invention relates to a nucleic acid construct comprising: a nucleotide sequence encoding a heavy chain variable domain, a protease cleavable peptidic linker and a masking moiety; and/or a nucleotide sequence encoding a light chain variable domain, a cleavable peptidic linker and a masking moiety;

wherein the nucleotide sequences are operably linked to an expression control sequence for expression in a host cell, preferably a mammalian host cell. Both the heavy chain and the light chain may be expressed from (different expression cassettes in) the same expression vector, or from different expression vectors.

Preferably, the expression control sequence includes a promoter and optionally other regulatory elements such as e.g. terminators, enhancers, polyadenylation signals, signal sequences for secretion and the like. Such nucleic acid constructs are particularly useful for the production of the activatable antibodies or antigen binding fragments thereof of the invention using recombinant techniques in which a nucleotide sequence encoding the activatable antibody is expressed in suitable host cells such as described in Ausubel et al., “Current Protocols in Molecular Biology”, Greene Publishing and Wiley-Interscience, New York (1987) and in Sambrook and Russell (2001) “Molecular Cloning: A Laboratory Manual (3rd edition), Cold Spring Harbor Laboratory, Cold Spring Harbor Laboratory Press, New York). As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

In a preferred embodiment, the nucleotide sequences defined above are codon optimized for the host cell wherein the activatable antibody or antigen binding fragment thereof according to the invention is produced.

In a preferred embodiment, the nucleic acid construct is a vector that is compatible with the host cell.

### Host Cell

In a further aspect, the invention pertains to a host cell comprising a nucleic acid construct as defined above. Preferably the host cell is a host cell for production of activatable antibody or antigen binding fragment thereof of the invention. The host cell may be any host cell capable of producing an antigen-binding protein of the invention, including e.g. a prokaryotic host cell, such as e.g., E. coli, or a (cultured) mammalian, plant, insect, fungal or yeast host cell, including e.g. CHO-cells, BHK-cells, human cell lines (including HeLa, COS, HEK293 and PER.C6), Sf9 cells and Sf+ cells. A preferred host cell for production of an activatable antibody or antigen binding fragment thereof of the invention is however a mammalian cell, more preferably, a HEK293 or a CHO cell.

### Manufacture

Masked antibodies or antigen binding fragments according to the invention can be made by recombinant DNA techniques known in the art for the production of recombinant antibodies or antigen binding fragments. In essence such methods involve the creation of a DNA construct (e.g., an expression vector) comprising the DNA coding sequences for the antibody or antigen binding fragment, as well as the coding sequences for the masking moieties and optionally for (a) peptidic linker(s) including optional spacer sequences, all in the appropriate order and orientation and with the usual expression control elements known in the art. Such constructs can be introduced in a suitable host (e.g., a bacterial, yeast or mammalian cell) for production of the masked antibodies or antigen binding fragments in cell culture in a suitable culture medium. The majority of monoclonal antibodies or antigen binding fragments produced for use as biotherapeutics are produced in mammalian cell cultures such as Chinese hamster ovary (CHO) cells, stably expressing the antibody or antigen binding fragment. The antibodies or antigen binding fragments can be harvested and purified from the cell culture. Thus, a further aspect of the invention relates to a method for producing an activatable antibody or antigen binding fragment thereof according to the invention, the method comprising the step of culturing a host cell as defined above under conditions conducive to expression of the activatable antibody or antigen binding fragment thereof. The method preferably comprise the steps of: (a) culturing a host cell as defined above under conditions conducive to expression of the activatable antibody or antigen binding fragment thereof; and optionally (b) purifying the activatable antibody or antigen binding fragment thereof from at least one of the host cell and the culture medium. Suitable conditions may include the use of a suitable medium, the presence of a suitable source of food and/or suitable nutrients, a suitable temperature, and optionally the presence of a suitable inducing factor or compound (e.g. when the nucleotide sequences of the invention are under the control of an inducible promoter); all of which may be selected by the skilled person. Under such conditions, the amino acid sequences of the invention may be expressed in a constitutive manner, in a transient manner, or only when suitably induced. The activatable antibody or antigen binding fragment thereof of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence of the invention) and/or preparative immunological techniques (i.e. using antibodies against the antigen-binding protein to be isolated).

### Use

In a further aspect, the present invention relates to the use of an antigenic peptide and a sdAb specifically binding to the antigenic peptide as part of a mask in an activatable antibody or an antigen binding fragment thereof according to the invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The word “about” or “approximately” when used in association with a numerical value (e.g., about 10) preferably means that the value may be the given value more or less 1% of the value.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature (RT) to about 37° C. (from about 20° C. to about 40° C.), and a suitable concentration of buffer salts or other components.

All patent and literature references cited in the present specification are hereby incorporated by reference in their entirety.

The following Examples are offered for illustrative purposes only, and are not intended to limit the scope of the present invention in any way.

### EXAMPLES

### Example 1: Transient Expression, Purification and HMW (%) Determination of (Masked) Antibodies (Materials and Methods)

### Design of Masked Antibody Chains

The masked antibody chains were assembled by joining the HAVT20 leader sequence (Boel et al., 2000, J. of Immunol. Meth., 239, 153-166, (SEQ ID NO: 43) to the N-terminus of the masking moiety comprising the BC2-nanobody (or variants thereof) or the BC2T peptide (or variants thereof) followed by a linker containing substrate sequence for Matrix metalloproteinase-2 (MMP2) linker sequence L9 (SEQ ID NO: 11) or L13 (SEQ ID NO: 12), which is a scrambled version of L9 not cleavable by MMP2), optionally flanked by spacer sequences (Qg: SEQ ID NO: 9 or Qv: SEQ ID NO: 10), and finally followed by either the heavy chain (HC) or light chain (LC) of anti-HER2 antibody trastuzumab (HC: SEQ ID NO: 21, HC-P41C SEQ ID NO: 22, LC: SEQ ID NO: 23) anti-EpCAM antibodies ING-1 (HC: SEQ ID NO: 24, HC-P41C SEQ ID NO: 25, LC: SEQ ID NO: 26) or adecatumumab (HC: SEQ ID NO: 27, HC-P41C SEQ ID NO: 28, LC: SEQ ID NO: 29), anti-TROP2 antibody sacituzumab (HC: SEQ ID NO: 30, HC-P41C SEQ ID NO: 31, LC: SEQ ID NO: 32), anti-TF antibodies MORAb-066 (HC: SEQ ID NO: 33, HC-P41C SEQ ID NO: 34, LC: SEQ ID NO: 35) or tisotumab (HC: SEQ ID NO: 36, HC-P41C SEQ ID NO: 37, LC: SEQ ID NO: 38), anti-CD137 antibody urelumab (HC: SEQ ID NO: 39, LC: SEQ ID NO: 40), or anti-CTLA4 antibody tremelimumab (HC: SEQ ID NO: 41, LC: SEQ ID NO: 42).

The BC2-Nb sequence is reflected in SEQ ID NO: 4, while its CDR3 sequence is depicted in SEQ ID NO: 5, variant CDR3 sequences are depicted in SEQ ID NO: 6-8. The BC2T peptide sequence is depicted in SEQ ID NO: 1, the sequence of BC2T peptide variants in SEQ ID NO: 2-3. Optionally, 7 amino acid residue spacer sequences QGQSGQG (Qg, SEQ ID NO: 9) or QVQLVES (Qv, SEQ ID NO: 10) were included in the masking moiety comprising the BC2T peptide, in front of (at N-terminus of) the BC2T sequence, enabling efficient signal peptide cleavage and enhancing expression of the antibody chain. Cleavage of the leader sequence corresponded to the predicted cleavage site using the SignalP program https://services.healthtech.dtu.dk/service.php?SignalP, Bendtsen, Jannick Dyrlov, et al. “Improved prediction of signal peptides: SignalP 3.0.” Journal of molecular biology 340.4 (2004): 783-795.).

To enable site-specific conjugation for the development of antibody-drug conjugates, a single cysteine residue was introduced to the heavy chain variable domain by replacing the residue at position 41 according to Kabat numbering for a cysteine residue. The resulting masked heavy chain and light chain amino acid sequences were back-translated into a cDNA sequence and codon-optimized for expression in human cells (Homo sapiens). The heavy chain and light chain cDNA constructs were chemically synthesized by and obtained from a commercial source (Geneart, Thermo Fisher Scientific).

### Expression Vector Construction

For expression of the antibody chains, the mammalian expression vector pcDNA3.4-TOPO (Invitrogen, Thermo Fisher Scientific) was used, which contains a CMV:BGHpA expression cassette. The heavy chain (HC) or the light chain (LC) genes were synthesized by synthetic oligo assembly and/or PCR products and individually cloned into pcDNA3.4-TOPO. After transfer to E. coli K12 DH10B T1R and expansion, large-scale production of the antibody chain expression vector for transfection was performed using the EndoFree Plasmid Maxi kit according to the manufacturer's instructions (Qiagen).

### Transient Expression in Mammalian Cells

Commercially available Expi293F cells (Gibco, Thermo Fisher Scientific) were co-transfected with the antibody chain expression vectors prepared as explained above using the FectoPRO transfection agent (Polyplus-transfection) according to the manufacturer's instructions, as follows: one day prior to transfection cells were seeded at 1×10⁶ viable cells (VC)/mL in 270 mL Expi293 Expression medium; the day after seeding, 240 μg of the antibody chain expression vectors (1:1 ratio) was combined with 240 μL of FectoPRO transfection agent and added to the cells. The cells were cultured according to the manufacturer's instructions (Gibco, Thermo Fisher Scientific). Small scale batches were obtained by linear scalability and thereby using equal volume ratios, as described above. Six days post transfection, the cell culture supernatant was harvested by centrifugation at 4,000 g for 15 minutes and filtering the clarified harvest over MF75 filters (Nalgene). For smaller batches (3 mL) the filtration step was omitted. Antibody concentrations were determined by ForteBio Octet QK384 using Protein A Biosensors and trastuzumab as calibrator according to manufacturer's instructions (Sartorius).

### Purification, and Titer and HMW Determination of (Masked) Antibodies

The following procedure was used to purify the (masked) antibodies. The harvest obtained from the expression, comprising the (masked) antibodies produced in Expi293 cells was subjected to capture on a Protein A resin (column or plate) and after elution either adjusted to pH 5.5.-6.0 or rebuffered to a pH 6.0 buffer. The steps were performed as follows: The protein A step (MabSelect SuRe, Cytiva) started with sanitization of a resin (column or plate) with 0.1 M NaOH for 15 minutes, followed by a rinse with Purified Water (PW). The resin was then equilibrated with PBS at pH 7.4. The (masked) antibody containing harvest was loaded onto the resin at a ratio of <25 mg (masked) antibody per mL MabSelect SuRe. The resin was then washed with equilibration buffer (PBS pH 7.4), followed by a wash with 25 mM NaAc pH 5.0. The (masked) antibody was then eluted with 25 mM acetate pH 3.0. The concentration of the purified (masked) antibody was measured with Lambert-Beer's law using a theoretical extinction coefficient based on the amino acid sequence of the (masked) antibody. After elution the (masked) antibody was either adjusted to pH 5.5.-6.0 or rebuffered to a pH 6.0 buffer.

For purity and soluble protein aggregation analysis of the (masked) antibodies a Size Exclusion Chromatography (SEC) method with UV detection was used. The (masked) antibodies were diluted to a concentration of 1 mg/mL and injected on a TSKgel UP-SW3000 column with a TSKgel UPSW Direct Connect Guard Column inline using 400 mM NaCl, 150 mM PO₄ buffer pH 6.2 with 15% 2-propanol as the mobile phase. The percentage main peak (monomer) and high molecular weight (HMW) species (e.g., soluble aggregation) were determined based on peak area relative to the total peak area of all protein-related peaks.

For antibodies purified using a column, the integrity of the (masked) antibodies was checked with SDS-PAGE.

### Example 2: Production of Various (Half-) Masked Antibodies 2A: Masked Trastuzumab Antibody Variants (SET1: Masked Antibodies S101-S112)

To investigate if different variants of masked trastuzumab antibodies could be produced at acceptable titers, variants were produced differing in how the masking moieties were linked to the antibody. The masked antibodies were based on anti-HER2 antibody trastuzumab (Ab) or trastuzumab with a modified HC sequence, wherein the Proline amino acid on position 41 is replaced by a Cysteine (41C). All antibodies carried masking moieties comprising the BCT2 peptide (SEQ ID NO: 1 or BC2-Nb (SEQ ID NO: 4).

The cleavable sequence comprised the MMP2 cleavable sequence PLGLAG (SEQ ID NO: 13), flanked by GS rich regions at both ends, resulting in the follow sequence: GGGGSPLGLAGGGS (herein referred to as “L9” and reflected in SEQ ID NO: 11).

Antibodies differed in whether the masking moiety with the BC2-Nb was attached to the HC and the masking moiety with the BCT2 peptide to the LC or vice versa.

In Table 1 the production titer for various masked antibodies is reflected. The “HC” column in Table 1 reflects the various masking moieties (Nanobody BC2-Nb or peptide BCT2, preceded by a spacer sequence (Qg or Qv)) at the N-terminal site of the peptide, and linker (L9) on the Heavy Chains of masked antibodies made, while the “LC” column reflects the various masking moieties and linker on Light Chains that were combined with the heavy chains into a masked antibody. Antibodies, where both the HC as well as the LC contained the same BC2 nanobody masking moiety could also be produced at high titers.

It was found that, in general, production of antibodies with the masking moiety comprising the nanobody on the LC (antibodies S103, S104, S107, S108, and S 110) resulted in higher titers, and a lower percentage of HMW complexes was formed. In Table 1 the percentage HMW complex in the product is reflected for masked antibodies with various combinations of masking moieties. For comparison, some “half-masked” antibodies were produced as well, where only the HC or LC comprised a masking moiety. Antibodies where both the HC and the LC contained the same masking moiety are also included. Evidently, the half-masked antibodies and the antibodies with a double Nb mask did form less HMW complexes.

TABLE 1
Production yield (titer (μg/mL) and percentage HMW for various masked trastuzumab variants (SET-1)

| Ref | | HC with masking | LC with masking | | Titer | HMW |
|---|---|---|---|---|---|---|
| No: | Based on Ab | moiety | moiety | Info | (μg/mL) | (%) |
| S101 | trastuzumab | BC2-Nb-L9-HC | Qg-BC2T-L9-LC | masked | 134 | 14.7 |
| S102 | trastuzumab | BC2-Nb-L9-HC | Qv-BC2T-L9-LC | masked | 112 | 18.6 |
| S103 | trastuzumab | Qg-BC2T-L9-HC | BC2-Nb-L9-LC | masked | 197 | 8.4 |
| S104 | trastuzumab | Qv-BC2T-L9-HC | BC2-Nb-L9-LC | masked | 216 | 9.1 |
| S105 | trastuzumab (P41C) | BC2-Nb-L9- HC (P41C) | Qg-BC2T-L9-LC | masked | 121 | 10.5 |
| S106 | trastuzumab (P41C) | BC2-Nb-L9-HC (P41C) | Qv-BC2T-L9-LC | masked | 125 | 17.2 |
| S107 | trastuzumab | BC2-Nb-L9-HC | BC2-Nb-L9-LC | double-Nb | 157 | 5.3 |
| S108 | trastuzumab (P41C) | BC2-Nb-L9-HC (P41C) | BC2-Nb-L9-LC | double-Nb | 111 | 4.6 |
| S109 | trastuzumab (P41C) | BC2-Nb-L9-HC (P41C) | wt LC | half-mask (HC-Nb) | 257 | 2.6 |
| S110 | trastuzumab (P41C) | HC (P41C) | BC2-Nb-L9-LC | half-mask (LC-Nb) | 317 | 4.0 |
| S111 | trastuzumab (P41C) | HC (P41C) | Qg-BC2T-L9-LC | half-mask (LC- | 373 | 3.9 |
| | | | | peptide) | | |
| S112 | trastuzumab (P41C) | HC (P41C) | Qv-BC2T-L9-LC | Half-mask (LC- | 329 | 1.4 |
| | | | | peptide) | | |

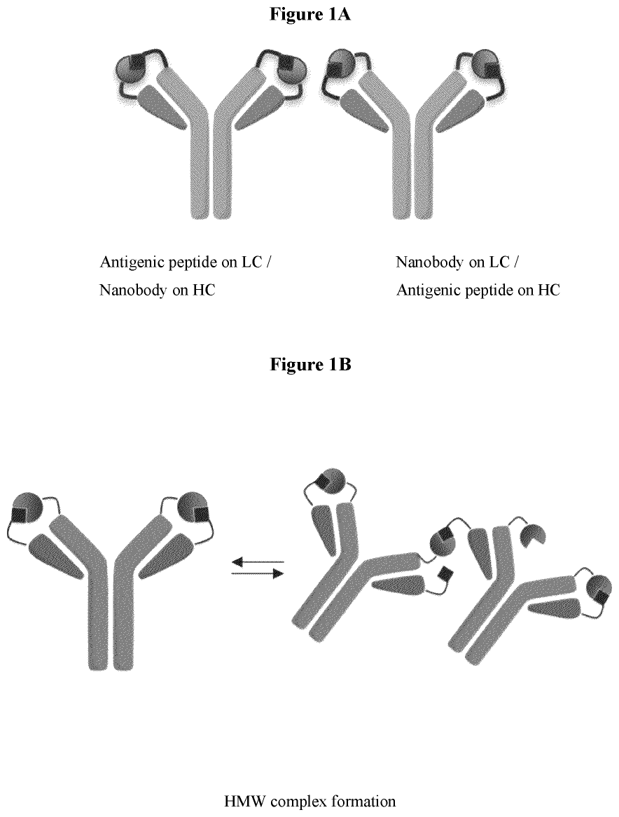

A schematic representation of antibodies with the Nb on the HC and peptide on the LC and vice versa is provided in FIG. 1A, while a schematic representation of the formation of HMW complexes between masked antibodies is presented in FIG. 1B.
2B: Comparison of Yield (Titer (μg/mL)) and HMW (%) after Production of Mutated and Wildtype BC2T/BC2-Nb Masked Trastuzumab

It was hypothesized that HMW formation could be correlated to the strength of the bond (affinity) between the masking moieties on the LC and HC. To test this hypothesis a set (SET-2) of antibodies was produced, using masking moieties based on the BC2-Nb and the BC2T peptide.

The antibody used was trastuzumab and masking moieties were attached to the N-termini of the HC and LC pairs using the L9 linker sequence also described in Example 1 for linkage of the (mutated) BC2T peptide and the (mutated) BC2-Nb.

With the idea that the affinity of the bond between the nanobody and the peptide would be lowered and aggregation, resulting in High Molecular Weight (HMW) complexes, could be reduced, mutations were introduced in the amino acid sequence for the BC2-Nb and/or the BC2 peptide to create BC2/BC2T variants. The mutations tested are reflected in Table 2. The mutations in the Nb are in the CDR3 of the Nb. The CDR3 of the BC2-Nb is reflected in SEQ ID NO: 5. The mutated CDR3 sequences of the BC2-Nb variants are reflected in SEQ ID NO 6-8, as indicated in Table 2.

TABLE 2
Mutated BC2T peptide and BC2-Nb variants used in experiments

| BC2T peptide variants | BC2-Nb variants |
|---|---|
| BC2T-S8A (SA, SEQ ID NO: 2) | BC2_{Nb}-R106A (RA, SEQ ID NO: 6) |
| BC2T-S8T (ST, SEQ ID NO: 3) | BC2_{Nb}-R106E (RE, SEQ ID NO: 7) |
| | BC2_{Nb}-R106S (RS, SEQ ID NO: 8) |

Each masking moiety with either the BC2T peptide or a variant thereof was combined with each respective masking moiety with the BC2-Nb or a variant thereof, resulting in 3×4=12 combinations. All combinations were tested with either the Nb or the peptide masking moiety on the HC or LC resulting in a total of 12×2=24 different masked antibodies.

Results (titer (μg/mL) and HMW (%) after production) for all 24 masked antibodies with combinations of masking moieties tested are reflected in Table 3.

TABLE 3
Titer and HMW (%) obtained for SET2 antibodies with
mutations in Nb and/or antigenic peptide sequences

| | | | | | HMW |
|---|---|---|---|---|---|
| Ref. No. | Based on Ab: | HC with masking moiety | LC with masking moiety | Titer (μg/mL) | (%) |
| S101 | trastuzumab | BC2-Nb-L9-HC | Qg-BC2T-L9-LC | 146 | 6.9 |
| S201 | trastuzumab | Qg-BC2T-L9-HC | BC2-Nb(RA)-L9-LC | 191 | 0.7 |
| S202 | trastuzumab | Qg-BC2T-L9-HC | BC2-Nb(RE)-L9-LC | 207 | 2.0 |
| S203 | trastuzumab | Qg-BC2T-L9-HC | BC2-Nb(RS)-L9-LC | 195 | 0.8 |
| S204 | trastuzumab | Qg-BC2T(ST)-L9-HC | BC2-Nb-L9-LC | 230 | 5.1 |
| S205 | trastuzumab | Qg-BC2T(ST)-L9-HC | BC2-Nb(RA)-L9-LC | 199 | 0.7 |
| S206 | trastuzumab | Qg-BC2T(ST)-L9-HC | BC2-Nb(RE)-L9-LC | 208 | 2.1 |
| S207 | trastuzumab | Qg-BC2T(ST)-L9-HC | BC2-Nb(RS)-L9-LC | 221 | 1.0 |
| S208 | trastuzumab | Qg-BC2T(SA)-L9-HC | BC2-Nb-L9-LC | 230 | 3.1 |
| S209 | trastuzumab | Qg-BC2T(SA)-L9-HC | BC2-Nb(RA)-L9-LC | 222 | 0.6 |
| S210 | trastuzumab | Qg-BC2T(SA)-L9-HC | BC2-Nb(RE)-L9-LC | 213 | 2.6 |
| S211 | trastuzumab | Qg-BC2T(SA)-L9-HC | BC2-Nb(RS)-L9-LC | 237 | 0.9 |
| S212 | trastuzumab | BC2-Nb-L9-HC | Qg-BC2T(ST)-L9-LC | 170 | 4.9 |
| S213 | trastuzumab | BC2-Nb-L9-HC | Qg-BC2T(SA)-L9-LC | 154 | 6.0 |
| S214 | trastuzumab | BC2-Nb(RA)-L9-HC | Qg-BC2T-L9-LC | 136 | 1.7 |
| S215 | trastuzumab | BC2-Nb(RA)-L9-HC | Qg-BC2T(ST)-L9-LC | 132 | 2.0 |
| S216 | trastuzumab | BC2-Nb(RA)-L9-HC | Qg-BC2T(SA)-L9-LC | 135 | 2.6 |
| S217 | trastuzumab | BC2-Nb(RE)-L9-HC | Qg-BC2T-L9-LC | 125 | 7.4 |
| S218 | trastuzumab | BC2-Nb(RE)-L9-HC | Qg-BC2T(ST)-L9-LC | 132 | 5.7 |
| S219 | trastuzumab | BC2-Nb(RE)-L9-HC | Qg-BC2T(SA)-L9-LC | 127 | 11.7 |
| S220 | trastuzumab | BC2-Nb(RS)-L9-HC | Qg-BC2T-L9-LC | 135 | 1.9 |
| S221 | trastuzumab | BC2-Nb(RS)-L9-HC | Qg-BC2T(ST)-L9-LC | 147 | 2.1 |
| S222 | trastuzumab | BC2-Nb(RS)-L9-HC | Qg-BC2T(SA)-L9-LC | 142 | 3.0 |
| Anti-PSMA | SYD1030 (S41C) | HC (S41C) | LC | 104 | |

What is evident from Table 3, is that production of antibodies where the masking moiety with the Nb is on the LC (5201-5211) result in a higher production titer, than when the Nb is on the HC (5212-5222).

It is further apparent that mutations in the nanobody sequence and/or peptide sequence result in a lower percentage of HMW complex formation (presumably because the binding affinity between the masking moieties is lowered due to the mutations). See for example results for antibodies 5209, 5205, 5201, 5203, S211 and 5207. The effect can be seen for antibodies where the Nb is on the LC, but also for antibodies where the Nb is on the HC (especially for the BC2-Nb R10A and R106S mutants, such as S214 and 5220).

### 2C: Production of Masked Antibodies Against Variety of Targets (SET3)

To prove that masked antibodies according to the invention can be made based on antibodies against a wide variety of targets, different masked antibodies (SET-3) were produced. SET3 included mAbs targeting EpCAM, TROP2, Tissue Factor (TF), CD137 and CTLA4. The set included unmasked (naked) antibodies as well as masked antibodies and masked versions of the antibodies carrying a 41C mutation in their HC (to prepare for later use in site-directed linkage of linker-drug molecules for the production of masked antibody-drug conjugates).

All masked antibodies in SET3 carried the following masking moieties: The BC2-Nb was linked with linker sequence L9 to the N-termini of the LCs of each antibody, while the masking moiety on the N-termini of the HCs of each antibody carried the BC2T peptide preceded by the Qg sequence and linked by the L9 sequence to the HC.

### Results of Antibody Production

30 mL batches were produced for all masked antibodies.

HMW (%) was relatively low for all wt mAbs (0.6-2.7) as well as for both masked wt and 41C tisotumab (4.1 and 3.7, respectively).

Masked ING-1 mAb targeting EpCAM showed the highest HMW (%) (65.5). Masked wt and 41C-adecatumumab (also targeting EpCAM) also showed a relatively high HMW (%) (33.7 and 30.9, respectively), as reflected in Table 4.

### 2D: Production Yield (Mg) and HMW (%) of Half-Masked Antibodies Against Different Targets (EpCAM, TROP2, TF, CD137 and CTLA4)

Half-masked antibodies could also be produced as is evidenced by the data reflected in Table 5.

Antibodies against various targets were half-masked, meaning that a masking moiety comprising the BC2-Nb was linked to the LC of the antibody or a masking moiety comprising the BC2T antigenic peptide was linked to the HC of the antibody, using the L9 linking sequence. The Qg spacer sequence was included in front of the peptide sequence when linked to the HC.

For various antibodies two variants of masked HC were produced: one with the wildtype sequence for the antibody HC and one with a 41C HC mutation (the 41C mutation introduced a cysteine which can be used for site-specific linkage of linker-drug molecules when the antibody is used in an ADC).

TABLE 4
Production amount (mg) and HMW (%) of masked antibodies directed against various targets (SET3)

| Reef. | | | LC with masking | Yield | HMW |
|---|---|---|---|---|---|
| No. | Based on Ab | HC with masking moiety | moiety | (mg) | (%) |
| S300 | Anti-EpCAM: ING-1, IgG1/IgKC (US20050009097) | wt HC | wt LC | 3.7 | 1.14 |
| S301 | Anti-EpCAM: ING-1 | Qg-BC2T-L9-HC | BC2-Nb-L9-LC | 3.1 | 65.5 |
| S302 | Anti-EpCAM: ING-1 | Qg-BC2T-L9-HC (P41C) | BC2-Nb-L9-LC | 1.5 | 65.5 |
| S303 | Anti-EpCAM: adecatumumab (MT-201), human | wt HC | wt LC | 6.6 | 0.6 |
| | IgG1/IgKC (U.S. Pat. No. 7,976,842) | | | | |
| S304 | Anti-EpCAM: adecatumumab | Qg-BC2T-L9-HC | BC2-Nb-L9-LC | 4.6 | 33.7 |
| S305 | Anti-EpCAM: adecatumumab | Qg-BC2T-L9-HC (P41C) | BC2-Nb-L9-LC | 4.5 | 30.9 |
| S306 | Anti-TROP2: sacituzumab hRS7, humanized IgG1/ | wt HC | wt LC | 3 | 2.7 |
| | IgKC (U.S. Pat. 7,238,785) | | | | |
| S307 | Anti-TROP2: sacituzumab | Qg-BC2T-L9-HC | BC2-Nb-L9-LC | 5.4 | 9.4 |
| S308 | Anti-TROP2: sacituzumab | Qg-BC2T-L9-HC (P41C) | BC2-Nb-L9-LC | 2.9 | 5.2 |
| S309 | Anti-TF: MORAb-066, CNT0860, humanized | wt HC | wt LC | 5.5 | 0.7 |
| | IgG1/IgKC (U.S. Pat. 7,605,235) | | | | |
| S310 | Anti-TF: MORAb-066 | Qg-BC2T-L9-HC | BC2-Nb-L9-LC | 4.4 | 6.7 |
| S311 | Anti-TF: MORAb-066 | Qg-BC2T-L9-HC (P41C) | BC2-Nb-L9-LC | 6.1 | 6.9 |
| S312 | Anti-TF: tisotumab (TF-011), human IgG1/IgKC | wt HC | wt LC | 4.1 | 1.6 |
| | (U.S. Pat. 9,150,658) | | | | |
| S313 | Anti-TF: tisotumab | Qg-BC2T-L9-HC | BC2-Nb-L9-LC | 6.7 | 4.1 |
| S314 | Anti-TF: tisotumab | Qg-BC2T-L9-HC (P41C) | BC2-Nb-L9-LC | 4.8 | 3.7 |
| S315 | Anti-CD137 (4-1BB): urelumab, human IgG4/IgKC | wt HC | wt LC | 2.5 | 1.1 |
| | (WO2005/035584) | | | | |
| S316 | Anti-CD137: urelumab | Qg-BC2T-L9-HC | BC2-Nb-L9-LC | 3.2 | 15.5 |
| S317 | Anti-CTLA4: tremelimumab, human IgG2/IgKC | wt HC | wt LC | 3.6 | 1.5 |
| | (U.S. Pat. 6,682,736) | | | | |
| S318 | Anti-CTLA4: tremelimumab | Qg-BC2T-L9-HC | BC2-Nb-L9-LC | 0.20 | 0.6 |

TABLE 5
Production amount (mg) and HMW (%) for half-masked antibodies against different targets.

| Ref. No. | Based on Ab: | HC with masking moiety | LC with masking moiety | Yield (mg) | HMW (%) |
|---|---|---|---|---|---|
| HM18 | Anti-EpCAM: ING-1 | HC | BC2-Nb-L9-LC | 2.26 | 0.79 |
| HM19 | Anti-EpCAM: ING-1 | Qg-BC2T-L9-HC | LC | 3.38 | 1.09 |
| HM20 | Anti-EpCAM: ING-1 | Qg-BC2T-L9-HC (P41C) | LC | 2.62 | 0.58 |
| HM21 | Anti-EpCAM: adecatumumab | HC | BC2-Nb-L9-LC | 4.42 | 2.42 |
| HM22 | Anti-EpCAM: adecatumumab | Qg-BC2T-L9-HC | LC | 3.95 | 0.78 |
| HM23 | Anti-EpCAM: adecatumumab | Qg-BC2T-L9-HC (P41C) | LC | 3.69 | 0.67 |
| HM24 | Anti-TROP2: sacituzumab hRS7 | HC | BC2-Nb-L9-LC | 5.00 | 3.49 |
| HM25 | Anti-TROP2: sacituzumab hRS7 | Qg-BC2T-L9-HC | LC | 3.71 | 2.2 |
| HM26 | Anti-TROP2: sacituzumab hRS7 | Qg-BC2T-L9-HC (P41C) | LC | 2.26 | 0.5 |
| HM27 | Anti-TF: MORAb-066, CNT0860 | HC | BC2-Nb-L9-LC | 2.73 | 3.55 |
| HM28 | Anti-TF: MORAb-066, CNT0860 | Qg-BC2T-L9-HC | LC | 4.89 | 0.48 |
| HM29 | Anti-TF: MORAb-066, CNT0860 | Qg-BC2T-L9-HC (P41C) | LC | 4.65 | 0.68 |
| HM30 | Anti-TF: tisotumab (TF-011) | HC | BC2-Nb-L9-LC | 4.28 | 4.03 |
| HM31 | Anti-TF: tisotumab (TF-011) | Qg-BC2T-L9-HC | LC | 4.04 | 1.23 |
| HM32 | Anti-TF: tisotumab (TF-011) | Qg-BC2T-L9-HC (P41C) | LC | 3.71 | 0.86 |
| HM33 | Anti-CD137: urelumab | HC | BC2-Nb-L9-LC | 3.15 | 2.12 |
| HM34 | Anti-CD137: urelumab | Qg-BC2T-L9-HC | LC | 4.04 | 3.5 |
| HM35 | Anti-CTLA4: tremelimumab | HC | BC2-Nb-L9-LC | 3.55 | 2.6 |
| HM36 | Anti-CTLA4: tremelimumab | Qg-BC2T-L9-HC | LC | 4.94 | 1.19 |

### Example 3: Synthesis of Antibody-Drug Conjugates Based on (Masked or Half-Masked) Antibodies

ADCs based on masked antibodies, where the duocarmycin-based linker-drug LD2 (Elgersma et al., 2015, supra) was conjugated in a site-specific manner to the HC41 cysteine introduced in the antibody HC sequence, were synthesized as disclosed in Coumans et al., 2020, supra, according to the following protocol:

A solution of cysteine-engineered antibody (10-15 mg/mL, pH 5, 100 mM histidine)) was treated with 2-(diphenylphosphino)benzenesulfonic acid (diPPBS, 16-32 equiv, 10 mM in water), and the resulting mixture was incubated at RT for 16-24 h. The excess diPPBS was removed by a centrifugal concentrator (Vivaspin filter, 30 kDa cutoff, PES) using 4.2 mM histidine, 50 mM trehalose, pH 6, or by carbon filtration. The pH of the resulting antibody solution was raised to ˜7.4 using Tris (1 M in water, pH 8) or kept at pH 6 after which N,N-dimethylacetamide (DMA) was added followed by a solution of linker-drug (10 mM in DMA). The final concentration of DMA was 5-10%. The resulting mixture was incubated at RT in the absence of light for 2-3 h or overnight in the case of pH 6. In order to remove the excess of linker-drug, activated charcoal was added and the mixture was incubated at RT for at least 0.5 h. The charcoal was removed using a 0.2 μm PES filter, and the resulting ADC was formulated in 4.2 mM histidine, 50 mM trehalose, pH 6, using a Vivaspin centrifugal concentrator (30 kDa cutoff, PES).

By using the above-mentioned site-directed conjugation method the ADCs reflected in Table 6 were made, all prepared by site-directed conjugation, carrying linker-drug 2 (LD2), with a Drug-to-Antibody Ratio (DAR) of 2.

TABLE 6
ADCs based on masked/half-masked or unmasked antibodies.

| | | | | LC with masking |
|---|---|---|---|---|
| Ref. No | ADC | Ab | HC with masking moiety | moiety |
| ADC-S105 | ADC based on trastuzumab | S105 | BC2-Nb-L9-HC (P41C) | Qg-BC2T-L9-LC |
| | HC (P41C)-LD2, DAR = 2 | | | |
| ADC-S108 | ADC based on trastuzumab | S108 | BC2-Nb-L9-HC (P41C) | BC2-Nb-L9-LC |
| | HC (P41C)-LD2, DAR = 2 | | | |
| ADC-S109 | ADC based on trastuzumab | S109 | BC2-Nb-L9-HC (P41C) | wt LC |
| | HC (P41C)-LD2, DAR = 2 | | | |
| ADC-S110 | ADC based on trastuzumab | S110 | HC (P41C) | BC2-Nb-L9-LC |
| | HC (P41C)-LD2, DAR = 2 | | | |
| ADC-S111 | ADC based on trastuzumab | S111 | HC (P41C) | Qg-BC2T-L9-LC |
| | HC (P41C)-LD2, DAR = 2 | | | |
| ADC-S423 | ADC based on trastuzumab | S423 | Qg-BC2T-L9-HC (P41C) | BC2-Nb-L9-LC |
| | HC (P41C)-LD2, DAR = 2 | | | |
| ADC-S426 | ADC based on trastuzumab | S426 | Qg-BC2T-L13-HC (P41C) | BC2-Nb-L13-LC |
| | HC (P41C)-LD2, DAR = 2 | | | |
| ADC-RIT41C | Anti-CD20: rituximab | rituximab | HC (P41C) | Wt LC |
| | HC (P41C)-LD2, DAR = 2 | HC(P41C) | | |
| ADC- | ADC trastuzumab HC (P41C)- | trastuzumab | HC(P41C) | Wt LC |
| TRA41C | LD2, DAR = 2 | (P41C) | | |

### Example 4: Bio-Layer Interferometry (BLI) Experiments to Compare Target Binding of Masked and Naked (Unmasked) Antibodies

### Materials and Methods

### Enzymatic Demasking of Masked Antibodies/ADCs by Protease Treatment

A 24 mM APMA (p-aminophenylmercuric acetate, sigma-aldrich, A-9563) stock solution in DMSO was prepared. rhMMP2 (R&D systems, 902-MP, 0.1 mg/mL) was thawed to RT. 10 μL enzyme was activated with 0.44 μL fresh APMA solution at 37° C. for 1 hour. After incubation the activated enzyme solution was diluted with 329 μL TCNB (50 mM TRIS, 10 mM CaCl₂), 150 mM NaCl, 0.05% (w/v) BRIJ-35, pH 7.5) buffer to 3.0 ng/μL.

The masked mAbs or ADCs were diluted with TCNB buffer after which activated enzyme was added. Final conditions: 0.4 mg/mL substrate and a molar substrate to enzyme ratio of ˜300. The mixtures were incubated at 37° C. while gently shaking. After 2 hours the mixtures were cooled to RT and a sample was taken for SDS-page analysis, after which they were stored at −80° C. until further use.

### Bio-Layer Interferometry (BLI)

Biotinylated target protein was immobilized on SAX biosensor tips via biotin-streptavidin interaction, or on Ni-NTA for immobilization of His-tagged target protein. The masked antibodies were tested in a 50-fold dilution by dipping the immobilized SAX/Ni-NTA sensor tips in these analytes. To correct for differences in concentration between the analyte stocks, Protein A binding was estimated for the same samples. The binding levels of the masked antibodies (analytes) to the immobilized targets on the SAX or Ni-NTA tips and the Protein A sensor tips were measured after 10 seconds of association and used for analysis, since this is in the linear part of the binding curve and therefore proportional to the analyte concentration.

### Materials

384-well tilted bottom microplates Forte bio, Cat #18-5076
High Precision Streptavidin (SAX) dip and read sensor tips, Forte Bio, 18-5118
Protein A sensor tips, Forte Bio, 18-5012
HBS-EP+ buffer, GE Healthcare, 10×: BR-1006-69, #30964
Biocytin in HBS-EP+, 1 mg/mL (Example 4B)
Ni-NTA biosensors, Forte Bio, 18-5102 (Example 4B)
HBS-P+, GE Healthcare (Example 4B)

### Example 4A (SET2 Antibodies)

As target protein biotinylated Human HER2 (Acro Biosystems, Cat. HE2-H822R) immobilized on SAX biosensor tips.
Masked antibodies with concentrations indicated in Table 7.

TABLE 7
Masked antibodies tested for binding to
HER2, with respective concentrations

| | | | | Conc |
|---|---|---|---|---|
| Ref. | Based on | HC with masking | LC with | (mg/ |
| No. | Ab | moiety | masking moiety | mL) |
| S201 | trastuzumab | Qg-BC2T-L9-HC | BC2-Nb(RA)-L9-LC | 1.49 |
| S202 | trastuzumab | Qg-BC2T-L9-HC | BC2-Nb(RERE)-L9- | 2.98 |
| | | | LC | |
| S203 | trastuzumab | Qg-BC2T-L9-HC | BC2-Nb(RS)-L9-LC | 2.73 |
| S204 | trastuzumab | Qg-BC2T(ST)-L9-HC | BC2-Nb-L9-LC | 3.11 |
| S205 | trastuzumab | Qg-BC2T(ST)-L9-HC | BC2-Nb(RA)-L9-LC | 2.73 |
| S206 | trastuzumab | Qg-BC2T(ST)-L9-HC | BC2-Nb(RE)-L9-LC | 2.12 |
| S207 | trastuzumab | Qg-BC2T(ST)-L9-HC | BC2-Nb(RS)-L9-LC | 2.97 |
| S208 | trastuzumab | Qg-BC2T(SA)-L9-HC | BC2-Nb-L9-LC | 3.53 |
| S209 | trastuzumab | Qg-BC2T(SA)-L9-HC | BC2-Nb(RA)-L9-LC | 1.25 |
| S210 | trastuzumab | Qg-BC2T(SA)-L9-HC | BC2-Nb(RE)-L9-LC | 3.11 |
| S211 | trastuzumab | Qg-BC2T(SA)-L9-HC | BC2-Nb(RS)-L9-LC | 3.05 |
| S212 | trastuzumab | BC2-Nb-L9-HC | Qg-BC2T(ST)-L9-LC | 2.24 |
| S213 | trastuzumab | BC2-Nb-L9-HC | Qg-BC2T(SA)-L9-LC | 1.91 |
| S214 | trastuzumab | BC2-Nb(RA)-L9-HC | Qg-BC2T-L9-LC | 1.82 |
| S215 | trastuzumab | BC2-Nb(RA)-L9-HC | Qg-BC2T(ST)-L9-LC | 1.95 |
| S216 | trastuzumab | BC2-Nb(RA)-L9-HC | Qg-BC2T(SA)-L9-LC | 1.07 |
| S217 | trastuzumab | BC2-Nb(RE)-L9-HC | Qg-BC2T-L9-LC | 1.78 |
| S218 | trastuzumab | BC2-Nb(RE)-L9-HC | Qg-BC2T(ST)-L9-LC | 1.77 |
| S219 | trastuzumab | BC2-Nb(RE)-L9-HC | Qg-BC2T(SA)-L9-LC | 1.79 |
| S220 | trastuzumab | BC2-Nb(RS)-L9-HC | Qg-BC2T-L9-LC | 1.84 |
| S221 | trastuzumab | BC2-Nb(RS)-L9-HC | Qg-BC2T(ST)-L9-LC | 1.75 |
| S222 | trastuzumab | BC2-Nb(RS)-L9-HC | Qg-BC2T(SA)-L9-LC | 1.88 |
| S101 | trastuzumab | BC2-Nb-L9-HC | Qg-BC2T-L9-LC | 2.14 |
| S103 | trastuzumab | Qg-BC2T-L9-HC | BC2-Nb-L9-LC | 3.12 |

### Example 4B (SET3 Antibodies)

As target proteins the following reagents were immobilized on the sensor tips:
Biotinylated antigens EpCAM, TROP2 and CD-137 (4-1BB) immobilized on SAX biosensor tips:
   Biotinylated Human TROP2/TACSTD2 Protein, His Avitag, Cat. No. TR2-H82E5, 200 μg/mL
   Biotinylated Human EpCAM/TROP1 Protein, Avitag His tag, Cat. No. EPM-H82E8, 200 μg/mL
   Biotinylated Human CTLA4 Protein, His Avitag, Cat No. CT4-H82E1, 200 μg/mL
   Biotinylated Human 4-1BB/TNFRSF9 Protein, His Avitag, Cat No. 41B-H82E6, 200 μg/mL
Human Coagulation Factor III/Tissue Factor Protein, His Tag, Cat. No. TF3-H52H5, 600 μg/mL (the His-tag labeled Tissue Factor was immobilized on Ni-NTA biosensor tips)
The SET3 nanobody-peptide masked trastuzumab constructs tested are indicated in Table 10 at respective indicated concentrations.

TABLE 10
SET3 antibodies tested, with respective concentrations

| | | | | Conc. |
|---|---|---|---|---|
| Ref. | | | | (mg/ |
| Nr. | Ab. | HC | LC | mL) |
| S300 | Anti-EpCAM | wt HC | wt LC | 1.22 |
| S301 | ING-1 | Qg-BC2T-L9-HC | BC2-Nb-L9-LC | 1.27 |
| S302 | | Qg-BC2T-L9-HC(P41C) | BC2-Nb-L9-LC | 0.62 |
| S303 | Anti-EpCAM | wt HC | wt LC | 2.76 |
| S304 | adecatumumab | Qg-BC2T-L9-HC | BC2-Nb-L9-LC | 1.93 |
| S305 | | Qg-BC2T-L9-HC(P41C) | BC2-Nb-L9-LC | 1.88 |
| S306 | Anti-TROP2 | wt HC | wt LC | 1.25 |
| S307 | sacituzumab | Qg-BC2T-L9-HC | BC2-Nb-L9-LC | 2.24 |
| S308 | | Qg-BC2T-L9-HC(P41C) | BC2-Nb-L9-LC | 1.20 |
| S309 | Anti-TF | wt HC | wt LC | 2.28 |
| S310 | MORAb-066 | Qg-BC2T-L9-HC | BC2-Nb-L9-LC | 1.83 |
| S311 | | Qg-BC2T-L9-HC(P41C) | BC2-Nb-L9-LC | 2.56 |
| S312 | Anti-TF | wt HC | wt LC | 1.69 |
| S313 | tisotumab | Qg-BC2T-L9-HC | BC2-Nb-L9-LC | 2.57 |
| S314 | | Qg-BC2T-L9-HC(P41C) | BC2-Nb-L9-LC | 1.99 |
| S315 | Anti-CD137 | wt HC | wt LC | 1.04 |
| S316 | urelumab | Qg-BC2T-L9-HC | BC2-Nb-L9-LC | 1.33 |

### Example 4A: Reduced Binding of Masked SET2 Antibodies to HER2, when Compared to Trastuzumab

To test the effect of the mutations introduced in the masking moieties of SET2 masked antibodies on their masking efficiency, antibodies S201 to S222 were tested for their ability to bind to HER2 (the target antigen of the masked trastuzumab antibody). Their binding was compared to two masked antibodies from SET1 (S101 and S103), and the wt trastuzumab (TRA) as positive control (concentration of 21 mg/mL) and anti-PSMA SYD1030 (HC S41C) (aPSMA) as a negative control (concentration of 1 mg/mL). Experiments were performed as explained above under “Materials and Methods”.

### Results:

Tables 8 and 9 show binding levels as the result of potential binding of antibodies to the HER2 immobilized on SAX sensor tips and to Protein A sensor tips after 130s, when compared to a baseline. Results are reflected as a wavelength shift indicative of the optical thickness of the biolayer on the sensor tips in nm. The 130s report point corresponds to 10s of association, since a baseline is created in the first 120 seconds. The last column shows the quotient of HER2/concentration and this can be used to compare binding between the different nanobody-peptide trastuzumab constructs.

It was found that all masked trastuzumab constructs show significantly reduced binding to HER2 compared to unmasked trastuzumab, indicating that the modifications in the nanobody and/or peptide used in the masking moieties of the masked antibodies in SET2 do not negatively impact their ability to mask binding of the masked trastuzumab to its target HER2. Together with the data from Table 3 on production titers and HMW (%), these data show that masked antibodies that can be produced efficiently (reflected by a high titer and relatively low HMW (%)) still have favorable masking characteristics (meaning that binding of the masked antibodies to their target when compared to the positive control trastuzumab is significantly reduced).

TABLE 8
Binding of modified masked trastuzumab antibodies S201-
S211 (nanobody on light chain variable domain) compared
to SET1 antibodies S101 and S103 and unmasked trastuzumab.

| | HER2 binding | Protein A binding | HER2/ |
|---|---|---|---|
| | after 10 s of | after 10 s of association | Protein A * |
| Ref. No. | association (nm) | (nm) | 1000 |
| S201 | 0.0001 | 0.88 | 0.11 |
| S202 | 0.0064 | 1.6015 | 4.00 |
| S203 | 0.0042 | 1.5292 | 2.75 |
| S204 | 0.0099 | 1.8422 | 5.37 |
| S205 | 0.007 | 1.4564 | 4.816 |
| S206 | 0.0023 | 1.2763 | 1.80 |
| S207 | 0.005 | 1.7445 | 2.87 |
| S208 | 0.0095 | 1.927 | 4.93 |
| S209 | 0.0025 | 0.7699 | 3.25 |
| S210 | 0.0047 | 1.6942 | 2.77 |
| S211 | 0.0067 | 1.7332 | 3.87 |
| S101 | 0.0086 | 1.221 | 7.04 |
| S103 | 0.0116 | 1.7477 | 6.64 |
| TRA | 0.6076 | 1.8333 | 331.42 |
| SYD1030 | −0.0028 | 0.7621 | N/A |
| HBS-EP + | −0.0043 | −0.0018 | N/A |
| buffer | | | |

N/A = not applicable

TABLE 9
Binding of modified masked trastuzumab antibodies S212-S222
(Nb on HC) compared to SET1 antibodies S101 and S103
and unmasked trastuzumab.

| | HER2 binding | Protein A binding | HER2/ |
|---|---|---|---|
| | after 10 s of | after 10 s of association | Protein A * |
| Ref. No. | association (nm) | (nm) | 1000 |
| S212 | 0.0051 | 1.2852 | 3.97 |
| S213 | 0.007 | 1.1284 | 6.20 |
| S214 | 0.0043 | 1.0569 | 4.07 |
| S215 | 0.0055 | 1.1374 | 4.84 |
| S216 | 0.0002 | 0.6426 | 0.31 |
| S217 | 0.0017 | 0.9885 | 1.72 |
| S218 | 0.0039 | 1.0315 | 3.78 |
| S219 | 0.0019 | 0.9794 | 1.94 |
| S220 | 0.0071 | 1.0548 | 6.73 |
| S221 | 0.0084 | 1.0311 | 8.15 |
| S222 | 0.0084 | 1.1519 | 7.29 |
| S101 | 0.0058 | 1.2186 | 4.76 |
| S103 | 0.0095 | 1.7589 | 5.40 |
| TRA | 0.5911 | 1.9004 | 311.04 |
| SYD1030 | 0.001 | 0.7455 | N/A |
| HBS-EP + | 0.0005 | 0.0026 | N/A |
| buffer | | | |

N/A = not applicable

### Example 4B: Reduced Binding of Masked SET3 Antibodies to their Respective Targets when Compared to Naked (Unmasked) Antibodies

To investigate the masking efficiency of masked antibodies against a variety of targets, SET3 antibodies 5300-5317 were tested for their ability to bind to their respective targets using bio-layer interferometry (BLI). As explained in Example 2C, SET3 included mAbs targeting EpCAM (ING-1 and adecatumumab), TROP2 (sacituzumab), TF (MORAb-066 and tisotumab) and CD137 (urelumab). SET3 included naked (unmasked) antibodies as well as masked wt antibodies and masked antibodies carrying the P41C mutation in their HC (to prepare for later use in site-directed linkage of linker-drug molecules for the production of antibody-drug conjugates based on masked antibodies).

Experiments were performed as explained above under “Materials and Methods”.

### Results

Tables 11A-F shows binding levels reflected as a wavelength shift in nm, indicative of potential binding of antibodies to the immobilized target on SAX sensor tips or Ni-NTA sensor tips and to Protein A sensor tips after 70s. The 70s report point corresponds to 10s of association, since a baseline is created in the first 60 seconds. The last column shows the quotient of target binding/concentration and this can be used to compare binding between the different nanobody-peptide antibody constructs.

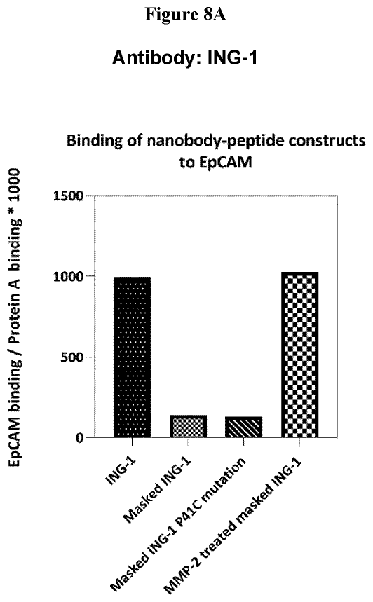

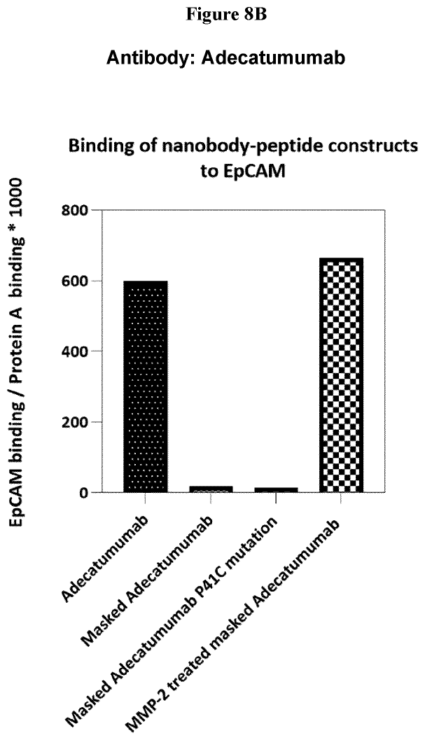

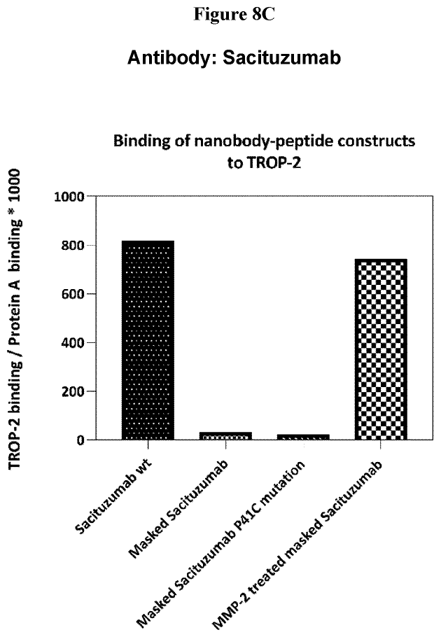

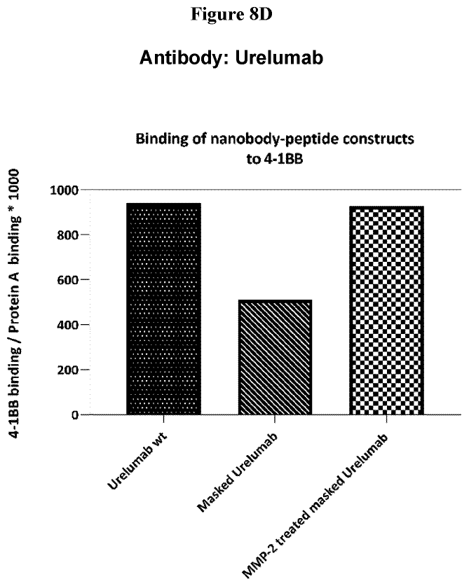

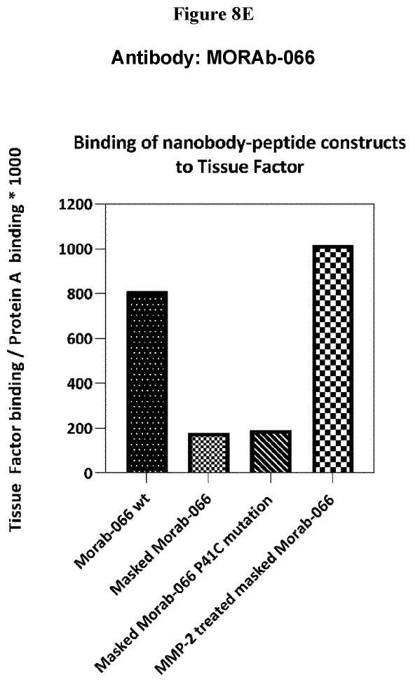

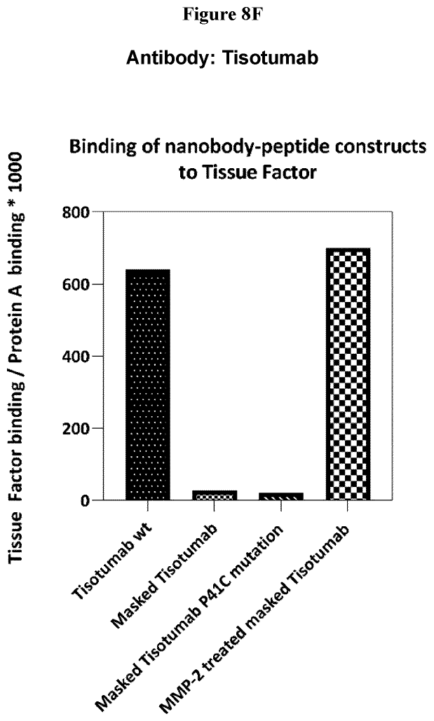

The results of the binding to its respective target of the naked, masked, masked (P41C) when tested, and MMP2-treated version of each antibody tested, corrected for differences in concentration (target binding/Protein A binding*1000) are also reflected in FIGS. 8A-F.

It was observed that the masked antibodies all showed significantly reduced binding to the target compared to the wildtype antibody.

The masked antibodies were also treated with MMP2 to analyze whether demasking occurs in the presence of MMP2. For all antibodies the binding levels of wildtype antibody were similar compared to the binding levels of the MMP2-treated masked antibodies thereby proving the concept of MMP2-cleavable nanobody-masked constructs.

TABLE 11A
Anti-EpCAM ING-1.

| | EpCAM binding | Protein A binding | EpCAM/ |
|---|---|---|---|
| | after 10 s of | after 10 s of | Protein A * |
| Sample | association (nm) | association (nm) | 1000 |
| S300 (naked) | 0.9472 | 0.9501 | 996.9 |
| S301 (masked) | 0.074 | 0.5409 | 136.8 |
| S302 (masked P41C) | 0.0354 | 0.2747 | 128.9 |
| MMP2-treated S301 | 0.5438 | 0.5301 | 1025.8 |
| SYD1030 | −0.004 | 0.8036 | N/A |
| HBS-EP + buffer | −0.004 | 0.0003 | N/A |

N/A = not applicable

TABLE 11B
Anti-EpCAM adecatumumab.

| | EpCAM binding | Protein A binding | EpCAM/ |
|---|---|---|---|
| | after 10 s of | after 10 s of | Protein A * |
| Sample | association (nm) | association (nm) | 1000 |
| S303 (naked) | 1.849 | 3.0881 | 598.8 |
| S304 (masked) | 0.0247 | 1.35 | 18.3 |
| S305 (masked P41C) | 0.0189 | 1.3076 | 14.5 |
| MMP2-treated S304 | 0.4748 | 0.7142 | 664.8 |
| SYD1030 | −0.004 | 0.8036 | N/A |
| HBS-EP + buffer | −0.004 | 0.0003 | N/A |

N/A = not applicable

TABLE 11C
Anti-TROP2 sacituzumab.

| | TROP2 binding | Protein A binding | TROP2/ |
|---|---|---|---|
| | after 10 s of | after 10 s of | Protein A * |
| Sample | association (nm) | association (nm) | 1000 |
| S306 (naked) | 0.7489 | 0.9155 | 818.0 |
| S307 (masked) | 0.0345 | 1.1091 | 31.1 |
| S308 (masked P41C) | 0.0154 | 0.6463 | 23.8 |
| MMP2-treated S307 | 0.4125 | 0.554 | 744.6 |
| SYD1030 | −0.0014 | 0.85 | N/A |
| HBS-EP + buffer | 0.002 | 0.0016 | N/A |

N/A = not applicable

TABLE 11D
Anti-CD137 (4-1BB) urelumab.

| | 4-1BB binding | Protein A binding | 4-1BB/ |
|---|---|---|---|
| | after 10 s of | after 10 s of | Protein A * |
| Sample | association (nm) | association (nm) | 1000 |
| S315 (naked) | 0.6195 | 0.6587 | 940.49 |
| S316 (masked) | 0.3372 | 0.6599 | 510.99 |
| MMP2-treated S316 | 0.5742 | 0.6181 | 928.98 |
| SYD1030 | 0.0001 | 0.8876 | N/A |
| HBS-EP + buffer | −0.0012 | 0.0051 | N/A |

N/A = not applicable

TABLE 11E
Anti-Tissue Factor (TF) MORAb-066.

| | TF binding after | Protein A | |
|---|---|---|---|
| | 10 s of | binding after | |
| | association | 10 s of | TF/Protein |
| Sample | (nm) | association (nm) | A * 1000 |
| S309 (naked) | 1.4549 | 1.793 | 811.43 |
| S310 (masked) | 0.178 | 0.9933 | 179.20 |
| S311 (masked P41C) | 0.2563 | 1.3335 | 192.20 |
| MMP2-treated S310 | 0.5268 | 0.5172 | 1018.56 |
| SYD1030 | −0.0063 | 0.9042 | N/A |
| HBS-EP+ buffer | 0.0002 | 0.0029 | N/A |

N/A = not applicable

TABLE 11F
Anti-Tissue Factor (TF) tisotumab.

| | TF binding | Protein A binding | |
|---|---|---|---|
| | after 10 s of | after 10 s of | TF/Protein A |
| Sample | association (nm) | association (nm) | * 1000 |
| S312 (naked) | 1.1241 | 1.7549 | 640.55 |
| S313 (masked) | 0.0465 | 1.7279 | 26.91 |
| S314 (masked P41C) | 0.0301 | 1.3681 | 22.00 |
| MMP2-treated S313 | 0.4414 | 0.6304 | 700.19 |
| SYD1030 | −0.0063 | 0.9042 | N/A |
| HBS-EP+ buffer | 0.0002 | 0.0029 | N/A |

N/A = not applicable

### Example 5: Cellular Binding and Cytotoxicity of Masked, Half-Masked and Demasked Antibodies

### Materials and Methods

### Determining Cellular Binding

Cellular binding of masked, half-masked, naked (unmasked) and demasked (MMP2-treated, according to the protocol described in Example 4.) antibodies and ADCs was studied in human HER2-positive tumor cell line SK-BR-3. Cells were used around 90% of confluence at the time of an assay, detached with Trypsin-Versene (EDTA) (Lonza) for 5-10 minutes, washed and adjusted to a concentration of 1×10⁶ cells/mL in ice-cold FACS buffer (PBS 1×, 0.1% v/w BSA, 0.02% v/v sodium azide (NaN₃)).

Staining was performed in 96-well round-bottomed microtiter plates using ice-cold reagents/solutions at 4° C. to prevent the modulation and internalization of surface antigens. 100,000 cells/well were added to 96-well plates (100 μL/well) and centrifuged at 300×g for 3 minutes. Supernatant was discarded and cells were stained for 30 minutes with 50 μlμl of each antibody or ADC. Serial dilutions were made in ice-cold FACS buffer. Cells were washed three times by centrifugation at 300×g for 3 minutes and resuspended in 50 μlμl 6000-times diluted secondary F(ab′)₂ goat anti-human IgG (Fc fragment specific) antibody APC-conjugated (Jackson ImmunoResearch).

Following a 30-minute incubation on ice, cells were washed again and resuspended in 150 μL ice-cold FACS buffer for FACS analysis. For analysis of the data, fluorescence intensities were determined by flow cytometry (BD Biosciences) and indicated as the median fluorescence intensity (MFI-Median).

Dose-response binding curves were fitted by nonlinear regression with a variable slope (four parameters) in GraphPad Prism version 9. EC₅₀ values were calculated in GraphPad Prism.

### Determining In Vitro Cytotoxicity

In vitro cytotoxicity of masked, unmasked, demasked and/or half-masked anti-HER2 ADCs was determined in human HER2-positive tumor cell line SK-BR-3 and human HER2-negative tumor cell line Jurkat NucLight Red. Cells in complete growth medium were plated in 96-well plates (90 μLμL/well) and incubated at 37° C., 5% CO₂ at the following cell density: 6500 SK-BR-3 and 3000 Jurkat NucLight Red, in 90 μl per well.

After an overnight incubation 10 μL of ADC was added. Serial dilutions of the ADC were made in culture medium. Cell viability was assessed after 6 days using the CellTiter-Glo® Luminescent Cell Viability Assay (Promega Corporation, USA) according to the manufacturer's instructions.

Percentage survival was calculated by dividing the measured luminescence for each ADC concentration with the average mean of untreated cells (only growth medium) multiplied with 100.

IC₅₀ values were calculated for each dose-response curve using the log(inhibitor) vs. response with a variable slope (four parameters) fit available in GraphPad Prism version 9.

### Example 5A: Cellular Binding to SK-BR-3 Cells of Masked and Demasked SET2 (S201-S211) Antibodies

Cellular binding experiments were performed with the antibodies of SET2. SET2 antibodies are reflected in Table 3 and include antibodies with mutations in the BC2-Nb and/or BC2T peptide sequence. Demasking with MMP2 and cellular binding was performed as described (M&M above).

The aim of this experiment was to investigate whether there is recovery of target binding after demasking of masked antibodies when compared to the target binding of naked (unmasked) trastuzumab. At the same time the effect of the different mutations in de BC2-Nb and/or BCT2 peptide sequence on the masking efficiency of the SET2 antibodies (5201-5211) was investigated and compared to S103 (S103 is masked with similar masking moieties as the SET2 antibodies, but without mutations in the BC2-Nb and BC2T peptide sequences).

The EC₅₀ shift in cellular HER2 target binding on SK-BR-3 cells as a result of demasking masked antibodies with MMP2, is reflected in Table 12, when compared to binding of naked (unmasked) trastuzumab. Reliable EC₅₀ values for target binding of masked Abs could not be determined because the dose response curves show no top (saturated binding was not reached). Table 12 shows the binding characteristics of all demasked SET2 antibodies and demasked S103 expressed as the binding EC₅₀, meaning the antibody concentration at 50% binding of the total binding, on HER2-positive SK-BR-3 cells and the EC₅₀ shift relative to naked (unmasked) trastuzumab.

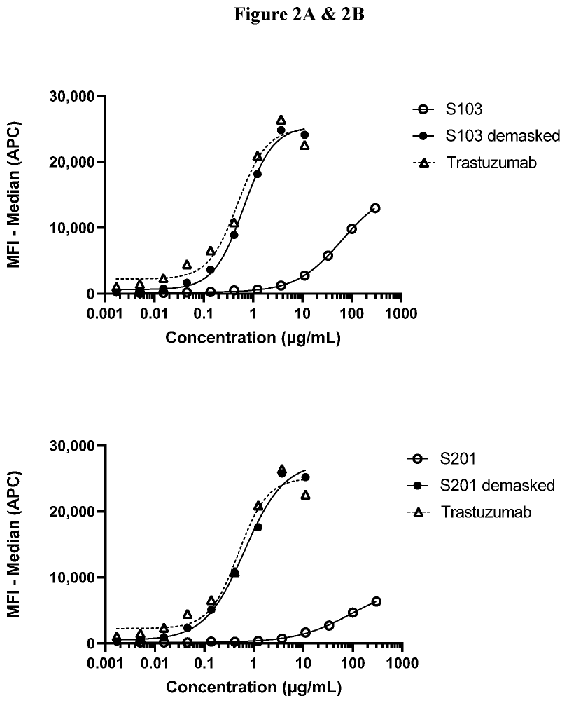

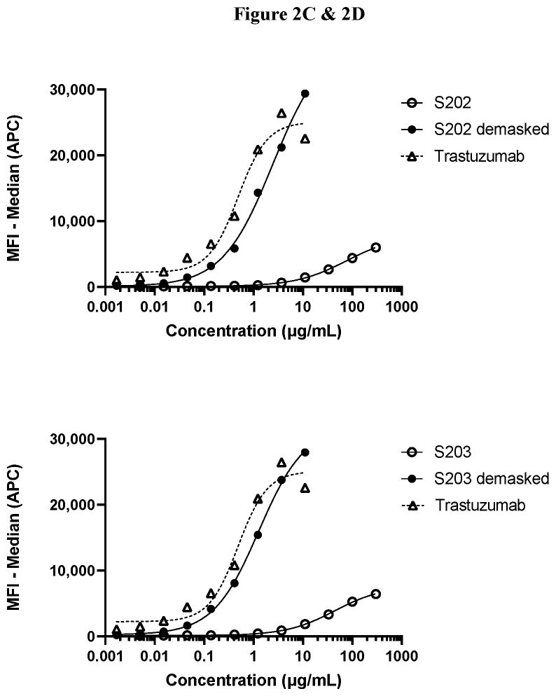

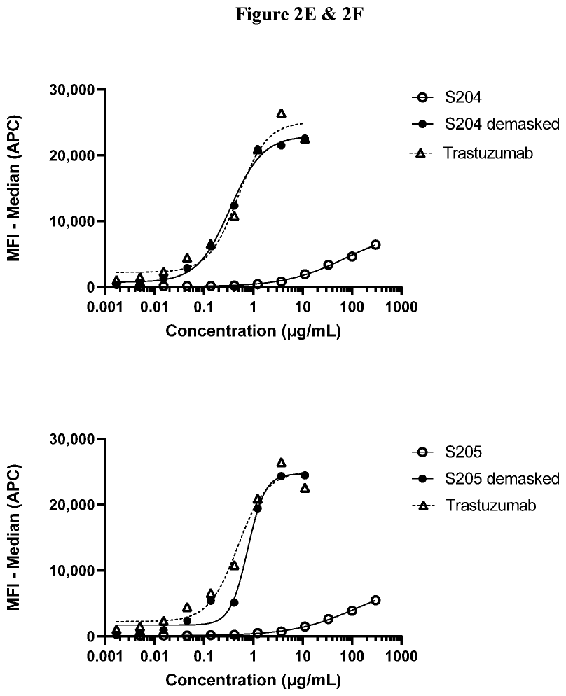

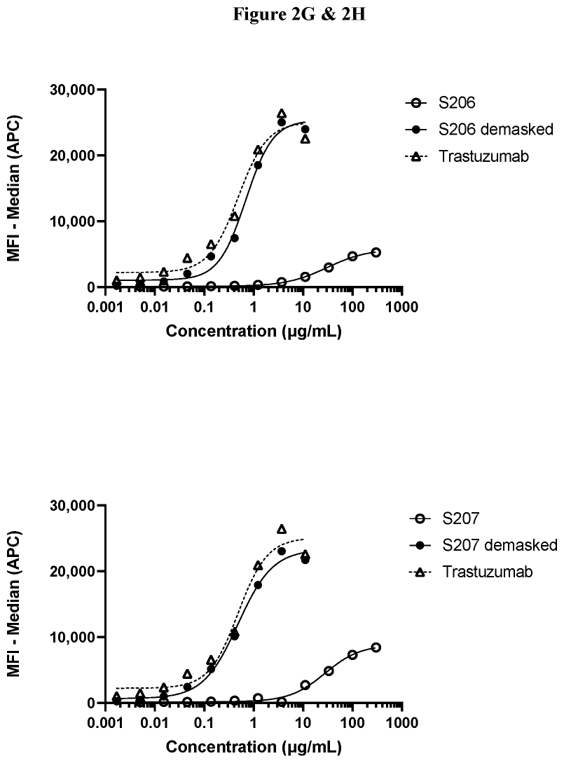

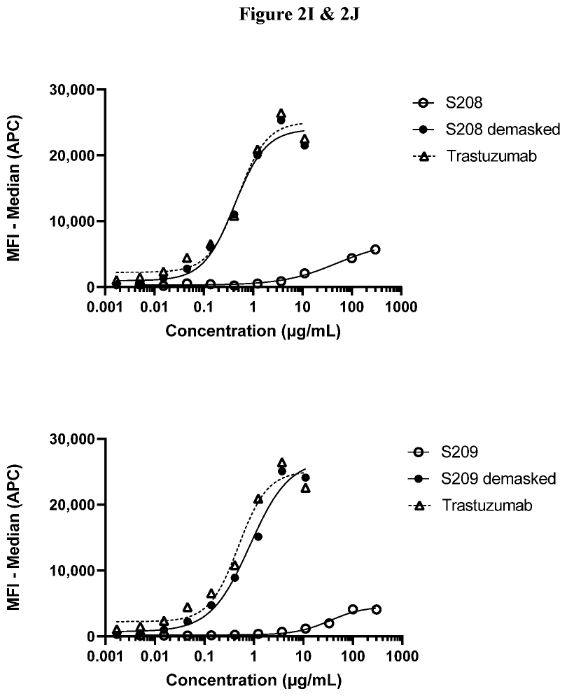

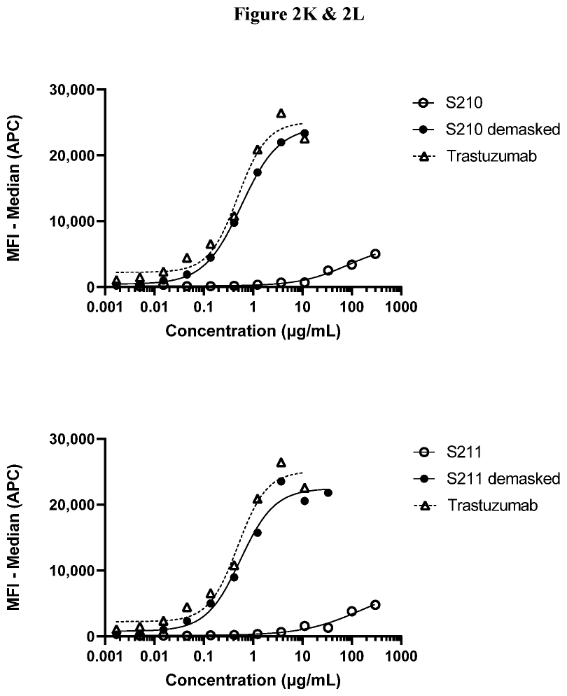

FIGS. 2A-2L show the dose-dependent binding of antibodies S103 and 5201-5211, when masked and after MMP2 treatment (demasked) on HER2-positive SK-BR-3 cells, compared to naked (unmasked) trastuzumab binding, as measured on a flow cytometer. Data show the MFI (median fluorescence intensity) from one experiment.

Dose-response curves reflected in FIG. 2A-2L, as well as results reflected in Table 12, show that antibodies were effectively demasked after MMP2 treatment, resulting in almost total recovery of HER2 target binding for most demasked antibodies (when compared to naked (unmasked) trastuzumab).

TABLE 12
EC₅₀ shift in cellular HER2 target binding on SK-BR-3 cells,
as a result of demasking masked antibodies with MMP2

| | | EC₅₀ shift(demasked Ab, |
|---|---|---|
| Ref. No. Ab | EC₅₀ | when compared to wt) |
| Naked (unmasked) trastuzumab | 0.49 | n.a. |
| Demasked S103 | 0.64 | 1.3 |
| Demasked S201 | 0.64 | 1.3 |
| Demasked S202 | 2.49 | 5.1 |
| Demasked S203 | 1.29 | 2.6 |
| Demasked S204 | 0.34 | 0.7 |
| Demasked S205 | 0.77 | 1.6 |
| Demasked S206 | 0.69 | 1.4 |
| Demasked S207 | 0.48 | 1.0 |
| Demasked S208 | 0.42 | 0.9 |
| Demasked S209 | 0.83 | 1.7 |
| Demasked S210 | 0.58 | 1.2 |
| Demasked S211 | 0.55 | 1.1 |

Because masked antibodies hardly bind to their target, even at very high concentrations, saturated binding was not achieved for masked antibodies (resulting in incomplete dose-response curves, as reflected in FIGS. 2A-2L) and reliable EC₅₀ values could not be determined.

When the concentrations where minimal binding can be observed are compared for masked, naked (unmasked) and demasked (MMP2-treated) antibodies, the difference in binding is apparent. Whereas minimal binding for unmasked or demasked antibodies is observed at concentrations as low as 0.04 μg/mL, the masked antibodies only showed minimal binding at concentrations in a range of 3.7 μg/mL (S103 and 5204) to as high as 33 μg/mL (S210). For most masked antibodies tested minimal binding could be observed only at a concentration as high as 11 μg/mL. If an estimated EC₅₀ was determined by hand with a calculator, values >300 μg/mL were obtained for binding of masked antibodies.

### Example 5B: Cytoxicity Induced on HER2-Positive SK-BR-3 Cells by Masked/Demasked ADC-S105

ADC-S105, as reflected in Table 6 is a masked trastuzumab (P41C) based ADC, based on masked antibody S105 mentioned in Table 1, which carries a masking moiety comprising the BC2 nanobody on its heavy chain (BC2-Nb-L9-HC (P41C)) and carries a masking moiety comprising the BC2T peptide, preceded by the Qg sequence on its LC (Qg-BC2T-L9-LC). Both masking moieties are linked to the antibody by MMP2 cleavable linker L9.

Cytotoxicity of masked and demasked (MMP2-treated) ADC-S105 was tested on HER2-positive SK-BR-3 cells to investigate whether the ADC was efficiently masked and whether the decrease in cell viability after demasking ADC-S105 with MMP2 proteases was comparable to a naked (unmasked trastuzumab 41C based) ADC. Therefore, for comparison, unmasked ADCs based on trastuzumab and rituximab (as isotype control) were also tested (ADC-TRA41C and ADC-RIT41C as reflected in Table 6).

Cell viability was measured using CellTiter-Glo™ (CTG) luminescent assay kit after 6 days of treatment.

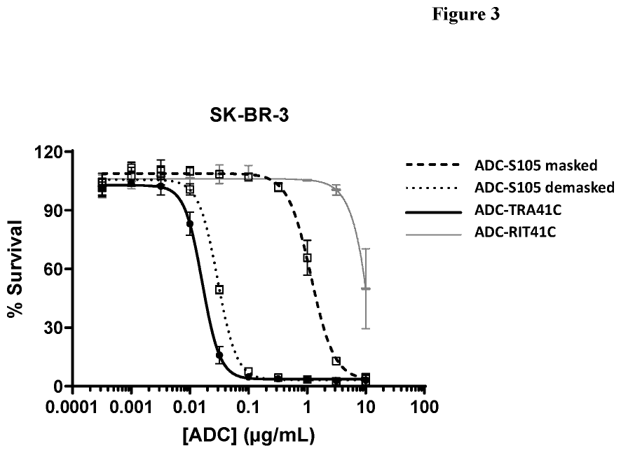

FIG. 3 shows dose-response curves in relation to cytotoxicity induced by ADC-S105, when it is masked and after MMP2 treatment (unmasked), as compared to ADC-TRA41C and isotype control ADC-RIT41C. Data show the percentage of survival of two experiments. Table 13 reflects the calculated IC₅₀, determined with CTG read out after 6 days treatment of HER2-positive SK-BR-3 cells with masked ADC-S105 and its demasked (MMP2-treated) ADC. The IC₅₀ potency shift induced by the masked concept is relative to ADC-TRA41C. The masked ADC-S105 shows a 73-fold IC₅₀ shift relative to ADC-TRA41C, which is a reflection of efficient masking of trastuzumab mediated cell killing. When the ADC-S105 is demasked after MMP2 protease treatment, an almost total recovery of induction of cytotoxicity is seen (demasked ADC-S105 shows a 2-fold IC₅₀ shift relative to ADC-TRA41C).

TABLE 13
IC₅₀ determined with CTG read out after 6 days
treatment of HER2-positive SK-BR-3 cells
with masked ADC-S105 and its unmasked ADC.

| Compound | IC₅₀ (μg/mL) | Potency shift |
|---|---|---|
| ADC-TRA41C | 0.016 | n/a |
| ADC-S105 masked | 1.17 | 73 (masked to naked) |
| ADC-S105 demasked | 0.030 | 2 (demasked to naked) |

### Example 6: Cellular Binding to HER2-Positive SK-BR-3 Cells by Masked/MMP2 Treated ADC-S423 and ADC-S426 (Non-Cleavable Linker L13), Compared to ADC-TRA41C

Cellular binding to HER2-positive SK-BR-3 cells of masked and MMP2-treated ADCs ADC-S423 and ADC-S426 were compared to ADC-TRA41C binding (+/− MMP2 treatment). Dose-dependent binding on HER2-positive SK-BR-3 cells was measured on a flow cytometer.

To investigate whether the cleavage of the L9-linker linking the masking moieties to the S423 antibody in ADC-S423 was MMP2 dependent, ADC-S426 was also tested. In the masked S426 antibody the same masking moieties as used in S423 are linked to the antibody by a scrambled version of the L9 linker (L13: GGGGSLALGPGGGS, SEQ ID NO: 12). L13 no longer contains the MMP2 cleavable site present in L9. (Trang et al. Nature biotechnology 37.7 (2019): 761-765, Jiang et al. Proceedings of the National Academy ofSciences 101.51 (2004): 17867-17872).

Both ADCs are reflected in Table 6. For comparison, naked (unmasked) ADC based on trastuzumab (ADC-TRA41C) was also subjected to MMP2 treatment.

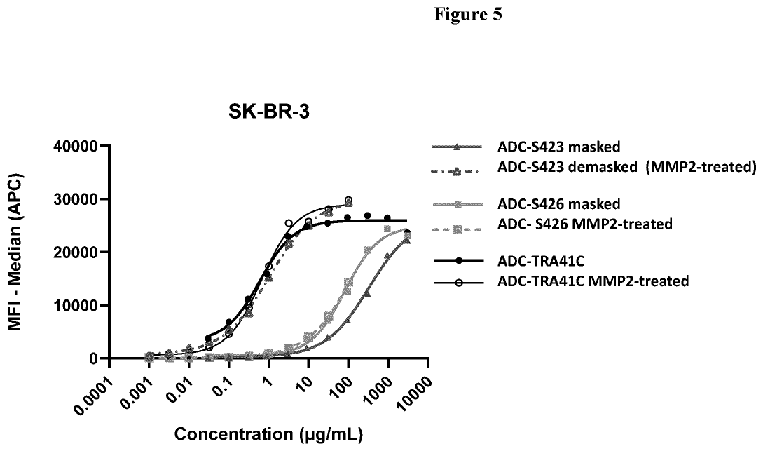

Results are shown in FIG. 5, showing the dose-dependent binding on HER2-positive SK-BR-3 cells for ADC-S423 and ADC-S426, masked and after MMP2 treatment (demasked), compared to ADC-TRA41C binding (+/− MMP2 treatment). Data show the MFI (median fluorescence intensity) from one experiment.

The masked ADC-S423 shows a 540-fold EC₅₀ shift relative to ADC-TRA41C, but a EC50<2, indicating almost total recovery of HER2 target binding when unmasked after MMP2 protease treatment. ADC-S426 shows a 137-fold EC₅₀ shift relative to ADC-TRA41C and no recovery of HER2 target binding after MMP2 protease treatment.

Results show that trastuzumab mediated binding on HER2-positive SK-BR-3 cells was effectively masked for both masked ADCs (ADC-S423 and ADC-S426). Binding was recovered for ADC-S423 after demasking by MMP2 treatment. The data also show that linker L13 was insensitive for MMP2, since binding was not recovered after MMP2 treatment.

### Example 7: Cytotoxicity of Masked/MMP2-Treated ADC-S423 and ADC-S426 on HER2-Positive SK-BR-3 Cells

To investigate the effect on cell viability of masked ADC-S423 after demasking with MMP2 proteases, cytotoxicity of masked and demasked (MMP2-treated) ADC-S423 and ADC-S426 was tested on HER2-positive SK-BR-3 cells and compared to naked (unmasked) trastuzumab based ADC-TRA41C and isotype control ADC-RIT41C. Cell viability was measured using CellTiter-Glo™ (CTG) luminescent assay kit after 6 days of treatment.

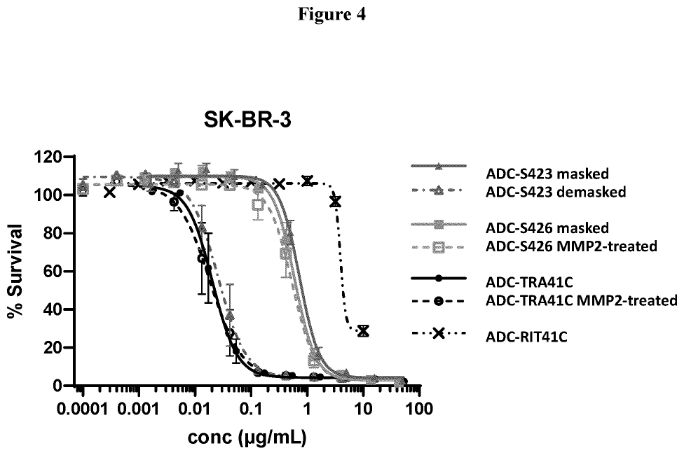

Results are reflected in FIG. 4 where cytotoxicity (reflected as % cell survival) induced by different concentrations of ADCs ADC-S423, ADC-S426, ADC-TRA41C, and isotype control ADC-RIT41C on HER2-positive SK-BR-3 cells.

The resulting IC₅₀ s determined with CTG read out after 6 days treatment of HER2-positive SK-BR-3 cells with masked ADC-S423 and ADC-S426 and the respective MMP2-treated ADCs are reflected in Table 14.

The masked ADC-S423 shows a 36-fold IC₅₀ shift relative to ADC-TRA41C, but an almost total recovery of induction of cytotoxicity when demasked after MMP2 protease treatment is seen (1.3-fold IC₅₀ shift of demasked ADC-S423 relative to ADC-TRA41C).

The scrambled linker in (ADC-)L026S426 cannot be cleaved by MMP2 as can be seen by a comparable 30- and 27-fold shift of ADC-S426 masked and MMP2-treated relative to ADC-TRA41C.

TABLE 14
Cellular binding (EC₅₀) and Cytotoxicity (IC₅₀) on
HER2-positive SK-BR-3 cells

| | Cellular Binding | Cytotoxicity |
|---|---|---|
| ADC | EC₅₀ (μg/mL) | IC₅₀ (μg/mL) |
| ADC-TRA41C | 0.60 | 0.020 |
| ADC-TRA41C MMP2-treated | 0.67 | 0.018 |
| ADC-S423 masked | 324 | 0.728 |
| ADC-S423 demasked | 1.04 | 0.026 |
| ADC-S426 masked | 82.01 | 0.594 |
| ADC-S426 MMP2-treated | >100* | 0.538 |

*DRC of ADC-S426 did not show a top because highest concentration tested was 100 μg/mL in the binding assay.

### Example 8: Cellular Binding to HER2-Positive SK-BR-3 Cells by Half-Masked Abs (A) as Well as Half-Demasked ADCs (B) and Cytotoxicity on HER2-Positive SK-BR-3 Cells of Half-(De)Masked ADCs (C)

To investigate whether a half-masked Ab or ADC is still able to bind its target and induce cell death as effective as the original (unmasked) Ab or ADC, cellular binding and cytoxicity of half-masked antibodies and ADCs was tested on HER2-positive SK-BR-3 cells. With “half-masked” in this experiment is meant that a masking moiety containing the Nb or the antigenic peptide is conjugated to the HC or LC of the “half-masked” antibody.

### 8A: Cellular Binding to HER2-Positive SK-BR-3 Cells by Half-Masked Ab

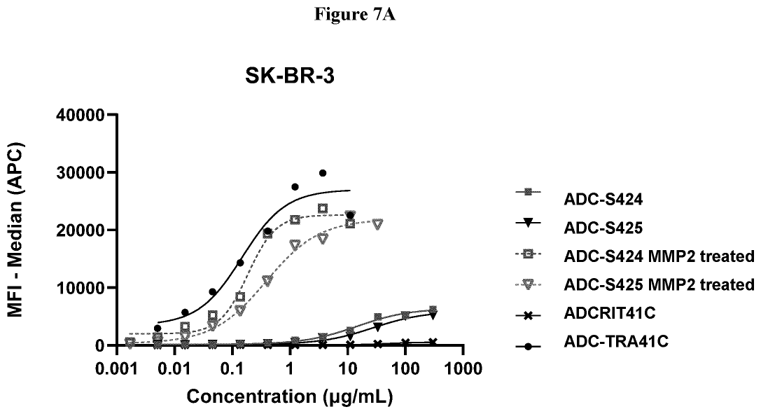

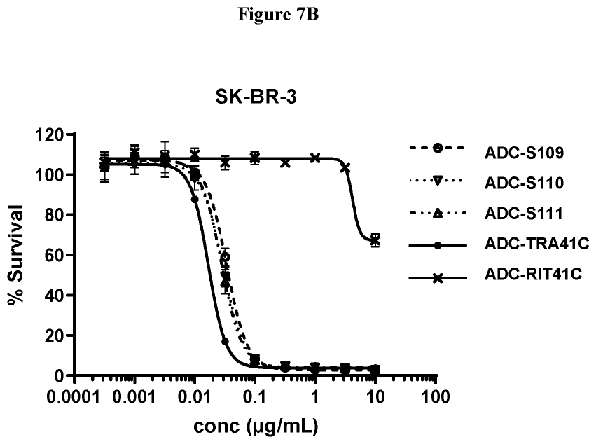

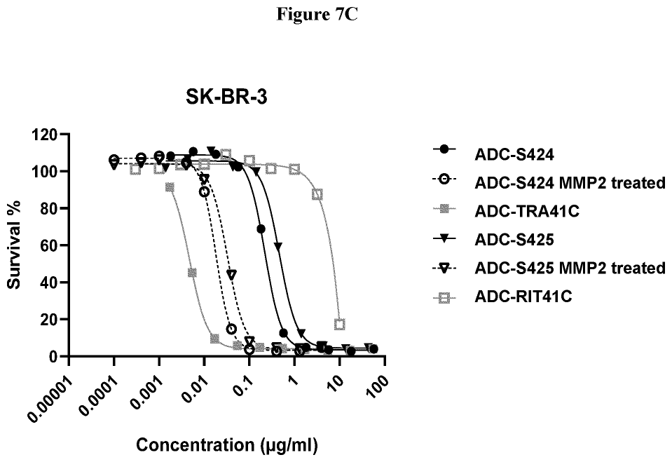

Cellular binding of half-masked antibodies S109 and S110 were compared to cellular binding of trastuzumab. Dose-dependent binding of antibodies on HER2-positive SK-BR-3 cells was measured on a flow cytometer. Data shown in FIG. 7A show the MFI (median fluorescence intensity) from two experiments. Table 15 shows the binding characteristics of the half-masked antibodies S109 and S110 expressed as the binding EC₅₀ on HER2-positive SK-BR-3 cells and the EC₅₀ shift relative to trastuzumab. Binding of half-masked Abs to the HER2 target was still possible although with a more than 4 times reduced potency compared to trastuzumab.

TABLE 15
Cellular binding of half-masked antibodies S109 and S110 compared to
trastuzumab on HER2-positive SK-BR-3 cells

| Ref. | | | | EC₅₀ | Potency |
|---|---|---|---|---|---|
| No.. | Ab | HC | LC | (μg/mL) | shift |
| TRA | trastuzumab | wt HC | wt LC | 0.430 | n/a |
| | IgG1/IgKC | | | | |
| S109 | trastuzumab | BC2-Nb-L9-HC | wt LC | 1.71 | 4 |
| | (P41C) | (P41C) | | | |
| S110 | trastuzumab | HC (P41C) | BC2-Nb-L9-LC | >30* | >70 |
| | (P41C) | | | | |

*Incomplete DRC

### 8B: Cellular Binding of (Half-Demasked) ADC-S424 and ADC-S425 Compared to ADC-TRA41C on HER2-Positive SK-BR-3 Cells.

ADCs, ADC-S424 and ADC-S425 are provided with a complete mask, wherein one masking moiety is attached with a non-cleavable linker (details in Table 16). In ADC-S424 the BC2T antigenic peptide is attached to the HC with a non-cleavable linker, while in ADC-S425 the Nb BC2 is attached to the LC with a non-cleavable linker. ADCs ADC-S424 and ADC-S425 are fully masked prior to MMP2 treatment, and “half-demasked” after MMP2 treatment. Cellular binding experiments on HER2-positive SK-BR-3 cells were performed as described above for the half-masked antibodies S109 and S110. MMP2 treatment of ADC-S424 and ADC-S425 was performed as described above.

For comparison, naked (unmasked) ADC based on trastuzumab (ADC-TRA41C) and the isotype control (ADC-RIT41C) were taken.

Results are shown in FIG. 7B, showing the dose-dependent binding on HER2-positive SK-BR-3 cells for ADC-S424 and ADC-S425, masked as well as after MMP2 treatment (half-demasked), compared to ADC-TRA41C and ADC-RIT41C binding. Data show the MFI (median fluorescence intensity) from one experiment.

The masked ADC-S424 and ADC-S425 hardly bound to the cells, reflected by a very flat dose response curve. What is apparent is that after MMP-2 treatment, where only one masking moiety was removed, cellular binding was largely restored for both half-demasked ADC-S424 and ADC-S425, although not entirely to the level of the naked ADC-TRA41C. Results are reflected in Table 16.

TABLE 16
Cellular Binding on HER2-positive SK-BR-3 cells of half-(de)masked
ADCs ADC-S424 and ADC-S425 compared to
ADC-TRA41C and isotype control ADC-RIT41C

| | | Potency shift relative to |
|---|---|---|
| Compound | EC₅₀ (μg/mL) | ADC-TRA41C |
| ADC-TRA41C | 0.14 | n/a |
| ADC-S424 Masked | 14.43 | 103 |
| ADC-S424 Demasked | 0.19 | 1.4 |
| ADC-S425 Masked | 27.52 | 197 |
| ADC-S425 Demasked | 0.39 | 2.8 |

### 8C: Cytotoxicity on HER2-Positive SK-BR-3 Cells of Half-(De)Masked ADCs Compared to ADC-TRA41C and Isotype Control ADC-RIT41C.

Cytotoxicity induced on HER2-positive SK-BR-3 cells by half-masked ADCs ADC-S109, ADC-S110, ADC-S111 (details in Table 6), and ADC-S424 and ADC-S425 was compared to ADC-TRA41C and isotype control ADC-RIT41C.

ADCs ADC-S109, ADC-S110 and ADC-S111 are based on antibodies that were only provided with one masking moiety (comprising the Nb BC2). These ADCs are therefore “half-masked”. ADCs ADC-S109, ADC-S110 and ADC-S111, were not treated with MMP2.

Cytotoxicity of ADC-S424 and ADC-S425 were determined prior-, as well as after MMP2 treatment. After MMP2 treatment, the masking moiety attached via the non-cleavable linker will remain attached to the antibody in ADC-S424 and ADC-S425 (and the ADCs are “half-demasked”).

Results are reflected in FIG. 7B (for ADC-S109, ADC-S110 and ADC-S111) and FIG. 7C (for ADC-S424 and ADC-S425). Cell viability was measured using CellTiter-Glo™ (CTG) luminescent assay kit after 6 days of treatment. Data show the percentage of survival of two experiments.

Table 17 shows the IC₅₀ determined with CTG read out after 6 days treatment of HER2-positive SK-BR-3 cells with the half-masked ADCs ADC-S109, ADC-S110, ADC-S111, ADC-S424, ADC-S425, as well as a ADC-TRA41C (ADCs are reflected in Table 6). The IC₅₀ potency shift induced by the half-masked concept is relative to ADC-TRA41C.

TABLE 17
Cytotoxicity of half-masked ADC-S109, ADC-S110, and
ADC-S111 on HER2-positive SK-BR-3 cells

| | | | | IC₅₀ | Potency |
|---|---|---|---|---|---|
| Ref. No. | ADC | | | (μg/mL) | shift |
| ADC- | ADC trastuzumab | wt HC(P41C) | wt LC | 0.017 | n/a |
| TRA41C | (P41C)- | | | | |
| | LD2, DAR = 2 | | | | |
| ADC- | ADC based on | BC2-Nb-L9- | wt LC | 0.034 | 2 |
| S109 | trastuzumab | HC(P41C) | | | |
| | HC (P41C)-LD2, | | | | |
| | DAR = 2 | | | | |
| ADC- | ADC based | HC(P41C) | BC2- | 0.029 | 1.7 |
| S110 | on trastuzumab | | Nb-L9- | | |
| | HC (P41C)-LD2, | | LC | | |
| | DAR = 2 | | | | |
| ADC- | ADC based on | HC(P41C) | Qg- | 0.028 | 1.6 |
| S111 | trastuzumab | | BC2T- | | |
| | HC (P41C)-LD2, | | L9-LC | | |
| | DAR = 2 | | | | |

After target binding, the half-masked ADCs ADC-S109, ADC-S110, ADC-S111 induce efficient cell death although with a 2 times reduced potency as compared to ADC-TRA41C, indicating that cleavage of one linker is enough to reach a cell killing potency almost similar to the unmasked ADC. No differences in binding or cytotoxicity potency was observed related to the differences in masking of half-masked Abs and ADCs ADC-S109, ADC-S110, ADC-S111.

Table 18 shows the IC₅₀ determined with CTG read out after 6 days treatment of HER2-positive SK-BR-3 cells with ADCs ADC-S424, ADC-S425, as well as a ADC-TRA41C (ADCs are reflected in Table 6). The IC₅₀ potency shift induced by the half-masked concept is relative to the corresponding masked ADC. (ADC-TRA41C has no top in the DRC and therefore no IC₅₀ can be determined).

TABLE 18
IC₅₀ on HER2-positive SK-BR-3 cells for ADCs ADC-S424, ADC-S425,
prior and after MMP treatment, as well as ADC-TRA41C.

| | | Potency shift relative to |
|---|---|---|
| Compound | IC50 (μg/mL) | corresponding masked ADC |
| ADC-TRA41C | n/a | n/a |
| ADC-S424 Masked | 0.22 | |
| ADC-S424 MMP2 treated | 0.02 | 11 |
| ADC-S425 Masked | 0.46 | |
| ADC-S425 MMP2 treated | 0.03 | 15 |

After MMP2 treatment, the half-demasked ADCs ADC-S424, ADC-S425 also induce efficient cell death, when compared to their fully masked (prior to MMP2 treatment) counterparts, although with a 11 times reduced potency as compared to ADC-TRA41C.

### Example 9: HMW Measurements in Production of Masked ADCs with Different DAR

To investigate the effect of mutations in the BC2-Nb sequence and the BC2T peptide sequence on HMW (%) in the production of ADCs with varying Drug-to-Antibody Ratios (DARs), ADCs with varying DAR were synthesized based on masked antibodies S103 and S211. Whereas S103 carries a mask based on the BC2-Nb and BCT2 peptide, S211 carries a mask wherein mutations were introduced in the BC2-Nb as well as the BCT2 peptide. S103 is detailed in Table 1, while S211 is detailed in Table 3. For convenience, both antibodies are again depicted in Table 19:

TABLE 19
Details of antibody S103 and S211.

| Ref | Based on | HC with masking | LC with masking |
|---|---|---|---|
| No: | Ab | moiety | moiety |
| S103 | trastuzumab | Qg-BC2T-L9-HC | BC2-Nb-L9-LC |
| S211 | trastuzumab | Qg-BC2T(SA)-L9-HC | BC2-Nb(RS)-L9-LC |

ADCs based on masked antibodies S103 and S211 carrying linker-drug LD2 (Elgersma et al., supra) with varying DAR were synthesized according to the following procedure:

A solution of masked antibody was mixed with buffer (4.2 mM histidine, 50 mM trehalose, pH 6), 1 M TRIS*HCl (pH 8), 25 mM EDTA solution and different aliquots of 2 mM TCEP solution. The different aliquots of TCEP were used to obtain a range of DAR after conjugation (different amounts of TCEP generates different amount of thiols available for conjugation and the amount of thiols determine the amount of conjugated linker-drug).

Final conditions were 2 mg/mL of antibody, 10 mM TRIS, 1 mM EDTA, TCEP (1 to 1.6 molar equivalents to antibody). N,N-dimethylacetamide (DMA) was added followed by a solution of linker-drug (10 mM in DMA, 3 molar equivalents of linker-drug to TCEP). The final concentration of DMA was 5%.

The resulting mixture was incubated at RT in the absence of light for 3 h after which a sample was taken for analysis by Size Exclusion Chromatography (SEC) for high molecular weight according to the following method: For soluble protein aggregation analysis of the ADCs a SEC method with UV detection was used. The (masked) antibodies were diluted to a concentration of 1 mg/mL and injected on a Waters Acquity UPLC BEH200 SEC using 100 mM PO₄ buffer pH 7.5 with 10% 2-propanol as the mobile phase. The percentage main peak (monomer) and high molecular weight (HMW) species (e.g., soluble aggregation) were determined based on peak area relative to the total peak area of all protein related peaks.

The average DAR for the ADCs was determined by HIC chromatography as described in Coumans et al, 2020, supra.

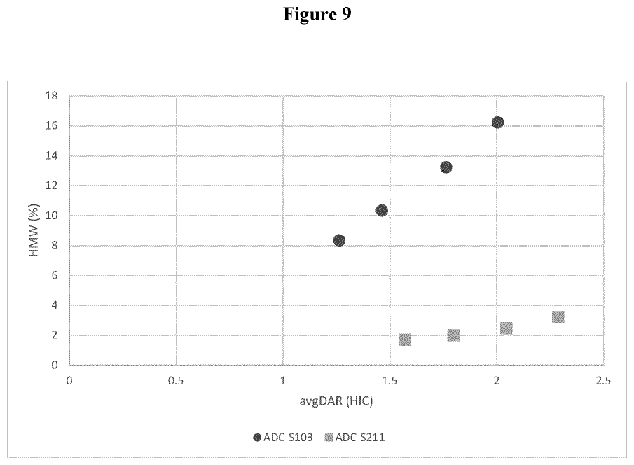

In FIG. 9 the high molecular weight versus average DAR of the tested ADCs is depicted. The tested range of DAR is reflected on the X-axis, while the percentage HMW in the obtained product is reflected on the Y-axis.

It is evident that the HMW (%) for ADC-5211 is lower over the tested DAR-range, when compared to ADC-S103.

### Example 10: Linker Selection (for Masked Anti-TF Antibodies)

Using the linkers as exemplified in Table 18 in all possible combinations, a set of 324 masked antibodies was produced, using the methods in analogy to what is described in Example 1. The masking moieties used in each case were a pair of masking moieties comprising a masking moiety with the BC2T SA mutant peptide linked to each LC, and a masking moiety with the BC2-Nb RS mutant nanobody linked to each HC of the anti-TF antibody tisotumab. In each antibody the sequence of the antigenic peptide was preceded by the Qg sequence.

In the below set of linkers in Table 20, linkers 14-25 all contain the protease cleavage site LSGRSDNH but differ in length, while linkers L26, L27, L9, L29, L7, and L30 all contain the PLGLAG protease cleavage site and likewise differ in length. The set of linkers reflected in the above table allows for testing of combinations of linkers with the same cleavage site for both masking moieties, while the length of the linkers differs. The set also allows for combinations of linkers that differ in cleavage site and/or length, and of course also encompasses combinations where exactly the same linker is used for both masking moieties. By testing all possible combinations (18×18=324 combinations), optimal linker pairs can be selected, such as for example for optimization of expression level and/or HMW (%).

For all antibodies produced the amount of antibody produced (μg/mL), the HMW (%) (percentage of total antibody lost due to formation of high molecular weight complexes) was determined.

TABLE 20
Set of linkers used in every possible
combination

| | | SEQ |
|---|---|---|
| | | ID |
| Linker configuration | Linker | NO: |
| LSGRSDNH-Ab | L14 | 14 |
| GGLSGRSDNH-Ab | L15 | 55 |
| SGGLSGRSDNH-Ab | L16 | 56 |
| GSGGLSGRSDNH-Ab | L17 | 57 |
| GGLSGRSDNHGGS-Ab | L18 | 58 |
| SGGLSGRSDNHGGS-Ab | L19 | 59 |
| GSGGLSGRSDNHGGS-Ab | L20 | 60 |
| GGSGGLSGRSDNHGGS-Ab | L21 | 61 |
| GGGSGGLSGRSDNHGGS-Ab | L22 | 62 |
| GGGGSGGLSGRSDNHGGS-Ab | L23 | 63 |
| GGGGSGGSGGLSGRSDNHGGS-Ab | L24 | 64 |
| GGGSSGGSGGSGGLSGRSDNHGGS-Ab | L25 | 65 |
| GGGSGGSPLGLAGGGS-Ab | L26 | 66 |
| GGSGGSPLGLAGGGS-Ab | L27 | 67 |
| GGGGSPLGLAGGGS-Ab | L9 | 11 |
| GGGSPLGLAGGGS-Ab | L29 | 68 |
| GGSPLGLAGGGS-Ab | L7 | 19 |
| GGPLGLAGGGS-Ab | L30 | 69 |

All antibodies were also tested for binding to their immobilized target Tissue Factor antigen in BLI, essentially as described in Example 4.

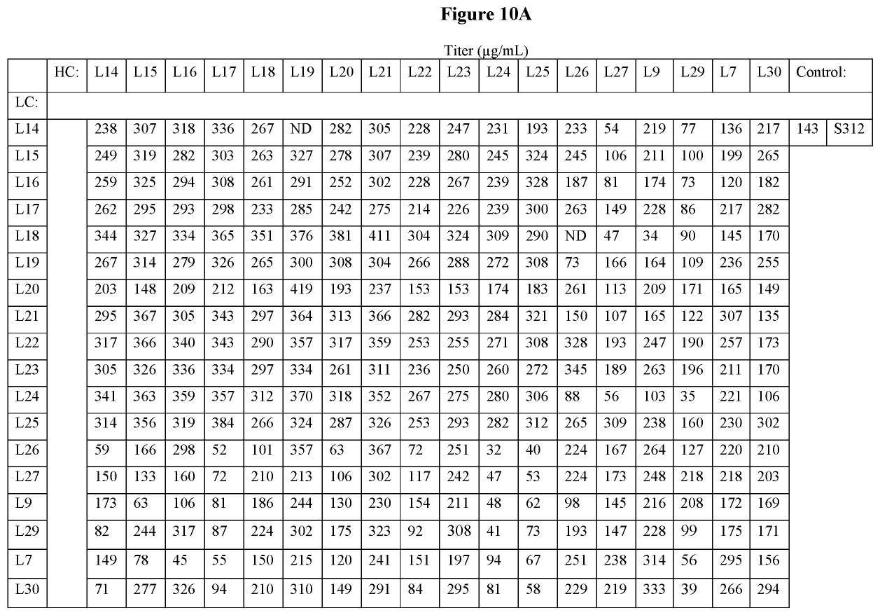

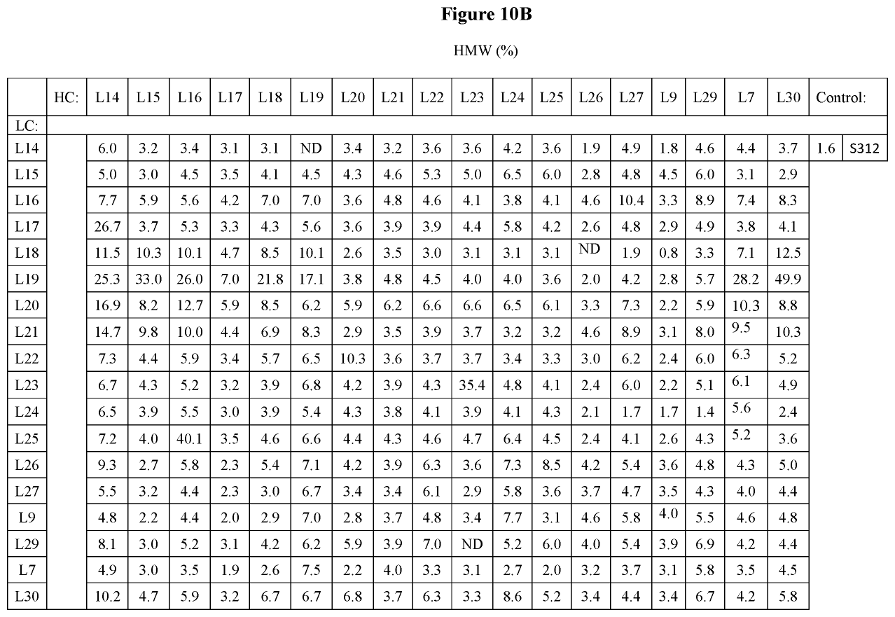

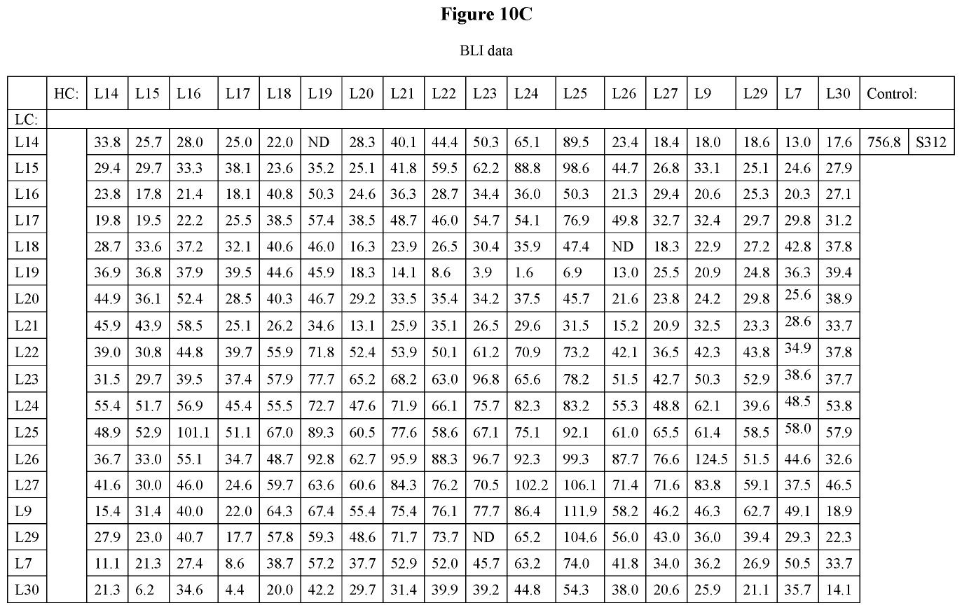

The obtained amount of antigen for each antibody is reflected in FIG. 10A.

The HMW (%) for each antibody is reflected in FIG. 10B.

The results of the BLI binding experiment are reflected as binding to tissue factor/binding to protein A*1000 in FIG. 10C.

Results for control antibody S312 (naked tisotumab), is provided in the tables reflected in FIGS. 10A-C as well.

By setting a pre-determined cut-off value for each measured parameter and combining the results of the 3 graphs, a selection of linker combinations for this particular antibody can be defined.

What is apparent is that the majority of the antibodies could be produced at expression levels above 100 μg/mL (264 out of 324). For the vast majority of antibodies the HMW (%) was below 10%, while for 86 linker combinations HMW (%) was below 5%, and for 13 even below 3%.

When the binding in the BLI experiment was compared to the binding of the parent antibodies, a significant reduction in binding was observed for all tested masked antibodies; the binding parameter for the parent antibody tisotumab was around 750, while for all masked antibodies the binding parameter was below 150, and for almost 50% of the antibodies even below 40.

When the 3 parameters are combined (expression level >100 μg/mL, HMW (%) below 4% and BLI binding parameter below 40), 52 antibodies still passed the test.

When the criterium for HMW (%) was put on less than 3% still 14 antibodies fulfilled all criteria. 10 out of these 14 could be produced at expression levels above 200 μg/mL.

Of course further comparisons can be done on a subset of the antibodies selected as explained above, for example in cell binding assays and/or by using cytotoxicity measurements.

The experiment shows that good results can be obtained with varying linker lengths, as well as with combinations of linkers with the same, or different protease cleavage sites.

### Example 11: ADCs of a Selection of 12 Antibodies (from Example 10)

Out of the masked antibodies tested in Example 10, 12 were selected for conjugation to DUBA to produce masked antibody drug conjugates (masked ADCs).
The selected antibodies and their linkers are reflected in Table 21.
ADCs were made on the basis of these antibodies by subjecting them to typical selective reduction and DUBA conjugation conditions, as described in Example 3.
For all masked antibodies only minor increases in HMW (%) were observed and the final formulated forms contained 3-8% HMW-species with DARs of 1.9. A non-masked ADC had 1.4% HMW and also a DAR of 1.9

TABLE 21
Selected conjugates based on masked 41C tisotumab antibodies

| ADC | LC-Linker | HC-linker | Protease combi | DAR | HMW (%) |
|---|---|---|---|---|---|
| ADC1 | L14 | L26 | Mat/MMP | 1.85 | 3.0 |
| ADC2 | L14 | L9 | Mat/MMP | 1.88 | 3.0 |
| ADC3 | L18 | L20 | Mat/Mat | 1.87 | 6.3 |
| ADC4 | L21 | L20 | Mat/Mat | 1.85 | 6.2 |
| ADC5 | L20 | L9 | Mat/MMP | 1.87 | 5.2 |
| ADC6 | L29 | L15 | MMP/Mat | 1.86 | 6.9 |
| ADC7 | L19 | L24 | Mat/Mat | 1.86 | 5.5 |
| ADC8 | L30 | L27 | MMP/MMP | 1.84 | 5.6 |
| ADC9 | L9 | L30 | MMP/MMP | 1.88 | 6.6 |
| ADC10 | L29 | L30 | MMP/MMP | 1.87 | 7.1 |
| ADC11 | L19 | L9 | Mat/MMP | 1.88 | 8.1 |
| ADC12 | L14 | L30 | Mat/MMP | 1.85 | 5.1 |
| ADC13 | n.a. | n.a. | n.a. | 1.85 | 1.4 |

### Example 12: In Vitro Characterization of 12 Selected Masked Anti-TF ADCs

The 12 ADCs reflected in Table 21 were tested in vitro.
The cytotoxicity of the 12 ADCs was tested essentially as described in example 5 under Materials and Methods. Because the antibody used in ADC1-ADC13 is anti-Tissue Factor (TF) antibody tisotumab, cytotoxicity was determined on TF expressing cell lines (instead of the HER2-positive tumor cell line used in Example 5). Cytotoxicity was studied using three cell lines: HCT-116, BxPC3 and FaDu that have antigen (TF) densities of ˜30.000, ˜200.000 and ˜400.000 respectively, which were classified as low, “moderate” and high. TF antigen expression on the surface of the human tumor cell lines was determined with antibody S312 (naked tisotumab) and secondary F(ab′)₂ goat anti-Human IgG (Fc fragment specific) antibody APC-conjugated by the Human IgG Calibrator kit (Biocytex, Marseille, France), according to the manufacturer's protocol.
There seemed to be a correlation between antigen expression levels and activity of masked ADCs:

In the low expressing HCT116 cell line, masked ADCs had overlapping curves with the isotype (non-binding) control ADCs, implying that at low levels of target density (for example healthy tissue), there is little target mediated activity to be expected.

In the moderate and high expressors BxPC3 and FaDu, the masked ADCs showed higher activity than the isotype controls (as reflected by the smaller IC₅₀ window between masked and demasked/half-demasked ADCs), implying that at higher antigen concentrations (for example in the tumor) the masked ADC can already start to exert anti-tumor effects (or at least display tumor accumulation) without being demasked.

Half-demasked ADCs have comparable IC₅₀ values as fully demasked ADC.

All ADCs (masked vs demasked) lack cytotoxicity on TF-negative BT474 and IncuCyte™ MJurkat NucLight™ Red (data not shown).

Between the individual masked ADCs (that differ from one another in the linkers used to link the masking moieties to the antibody) differences could be observed in activity and masking efficiency, although all ADCs were effectively masked.

Demasking and partial demasking led to regain of cell-killing capacity of the ADCs.

Results of cytotoxicity testing are reflected as IC₅₀ shifts in Table 22.

For each ADC (ADC1-12) IC₅₀ shift are provided compared to the dose response curve for the unmasked ADC13 (1^{st} column). This value provides an indication of the efficiency of the mask.

In the next column the IC₅₀ shift for demasked ADCs (protease treated) when compared to unmasked (naked) ADC13 is shown. Values here are very small, which is an indication that demasked ADCs bind to the target antigen in the same way as a naked ADC ((which never carried a mask to begin with) does.

The next two columns provide result of treatment of ADC1-12 with only one protease (either MMP2 or matriptase). For ADCs constructed with linker pairs wherein one linker comprises an MMP cleavage site, while the other linker comprises a matriptase cleavage site (ADC1, ADC2, ADC5, ADC6, ADC1 1 and ADC12), cleavage with either one or the other protease results in a half-(de)masked ADC, in that only one linker is cleaved and only one masking moiety thus disconnects from the antibody. Only for those half-(de)masked ADCs IC₅₀ shifts, when compared to the dose response curve for unmasked ADC13, are reflected in table 22. As can be seen in Table 22, half-(de)masking results in a dose response that is comparable to the dose response of the completely demasked, as well comparable to the unmasked ADC13 (reflected by the low values for the IC₅₀ shifts). This is a clear indication that after cleavage of only one of the linkers of a masked ADC, binding to the target antigen is already restored. From this table it is apparent that (half)-demasking of the masked ADCs result in a shift of the dose response curve, indicating that all masked ADCs were effectively masked and efficiently demasked.

TABLE 22
Results of cytotoxicity measurements, reflected as IC₅₀ shifts
for masked, half-(de)masked and demasked ADCs .:

| | | | | | |
|---|---|---|---|---|---|
| | | | | | Protease |
| | IC50 shift | IC50 shift | IC50 shift | IC50 shift | Cleavage |
| | Masked/ | demasked/ | matriptase/ | MMP2/ | sites in |
| ADC | ADC13 | ADC13 | ADC13 | ADC13 | linkers |

FaDu TF-high expression (~364.000 receptors/cell)

| | | | | | |
|---|---|---|---|---|---|
| ADC1 | 25 | 4.0 | 3.7 | 3.7 | Mat/MMP |
| ADC2 | 44 | 4.0 | 4.0 | 4.2 | Mat/MMP |
| ADC3 | 16 | 4.2 | — | — | Mat/Mat |
| ADC4 | 16 | 4.5 | — | — | Mat/Mat |
| ADC5 | 41 | 4.0 | 3.8 | 5.5 | Mat/MMP |
| ADC6 | 27 | 3.7 | 6.4 | 4.2 | MMP/Mat |
| ADC7 | 50 | 4.0 | — | — | Mat/Mat |
| ADC8 | 37 | 3.7 | — | — | MMP/MMP |
| ADC9 | 16 | 3.6 | — | — | MMP/MMP |
| ADC10 | 23 | 3.4 | — | — | MMP/MMP |
| ADC11 | 44 | 3.8 | 4.4 | 6.8 | Mat/MMP |
| ADC12 | 92 | 3.9 | 4.1 | 4.5 | Mat/MMP |

BxPC3 TF-moderate expression (217.000 receptors/cell)

| | | | | | |
|---|---|---|---|---|---|
| ADC1 | 111 | 4.9 | 3.7 | 4.3 | Mat/MMP |
| ADC2 | 160 | 5.6 | 3.5 | 5.3 | Mat/MMP |
| ADC3 | 66 | 6.2 | — | — | Mat/Mat |
| ADC4 | 70 | 6.3 | — | — | Mat/Mat |
| ADC5 | 159 | 4.5 | 3.7 | 6.8 | Mat/MMP |
| ADC6 | 112 | 4.7 | 7.6 | 4.0 | MMP/Mat |
| ADC7 | 183 | 5.7 | — | — | Mat/Mat |
| ADC8 | 195 | 4.8 | — | — | MMP/MMP |
| ADC9 | 141 | 4.1 | — | — | MMP/MMP |
| ADC10 | 179 | 4.1 | — | — | MMP/MMP |
| ADC11 | 182 | 5.2 | 4.1 | 8.2 | Mat/MMP |
| ADC12 | 279 | 6.0 | 3.3 | 5.4 | Mat/MMP |

HCT-116 TF-low expression (~31.000 receptors/cell)

| | | | | | |
|---|---|---|---|---|---|
| ADC1 | 677 | 11.6 | 32.0 | 16 | Mat/MMP |
| ADC2 | 860 | 15.9 | 16.4 | 19.6 | Mat/MMP |
| ADC3 | 468 | 16.6 | — | — | Mat/Mat |
| ADC4 | 483 | 25.1 | — | — | Mat/Mat |
| ADC5 | 1591 | 12.5 | 27.3 | 37.4 | Mat/MMP |
| ADC6 | 434 | 11.2 | 28.1 | 9.4 | MMP/Mat |
| ADC7 | 1357 | No DRC | — | — | Mat/Mat |
| ADC8 | 1442 | 12.2 | — | — | MMP/MMP |
| ADC9 | 648 | 11.5 | — | — | MMP/MMP |
| ADC10 | 759 | 9.0 | — | — | MMP/MMP |
| ADC11 | 1864 | 15.1 | 22.7 | 67.6 | Mat/MMP |
| ADC12 | 1065 | 15.2 | 21.0 | 44.3 | Mat/MMP |

### Example 13: In Vivo Testing of Masked ADCs

The xenograft studies were conducted at AAALAC (Association for Assessment and Accreditation of Laboratory Animal Care)-certified CROs, using well-established protocols developed and maintained at these CRO's.

The masked anti-TF ADCs 1-13 were tested in vivo, in a patient derived xenograft (PDX) model; the non-small cell lung cancer model LXFA629. The TF expression in this model was assessed using immunohistochemistry (IHC). Tumor tissues were fixated in formalin, embedded in paraffin and used for TF IHC using the rabbit monoclonal anti-TF antibody from Cell Signaling Technologies referenced #97438. The IHC staining showed a TF 2+ expression level.

LXFA629 tumors were cut into fragments (3-4 mm edge length) and unilaterally implanted SC in the flank of immune-deficient CES1c KO SCID mice. When tumor implant volumes approached the target range of 80 to 250 mm³, mice were randomized over the treatment groups (n=6 per group), aiming at comparable median and mean group tumor volumes. The mice were dosed the following day with a single IV dose injection of 2 or 2.4 mg/kg ADC through the tail vein in a buffer containing 4.2 mM Histidine, 50 mM Trehalose, pH 6.0 (buffer SOL092).

Anti-tumor activity of ADCs was assessed by measuring tumor sizes twice weekly till day 42. The length and width of the tumor were measured with calipers and the volume of the tumor estimated by the formula (Simpson-Herren et al., 1970):

$$Tumor volume = \frac{{Width}^{2} \times Length}{2}$$

When a tumor of an individual mouse reached 2000 mm3, it was sacrificed and the data of the complete group was not shown from that timepoint onwards.

All masked ADCs showed an efficacy that was in the same range as the efficacy of the unmasked anti-TF ADC.

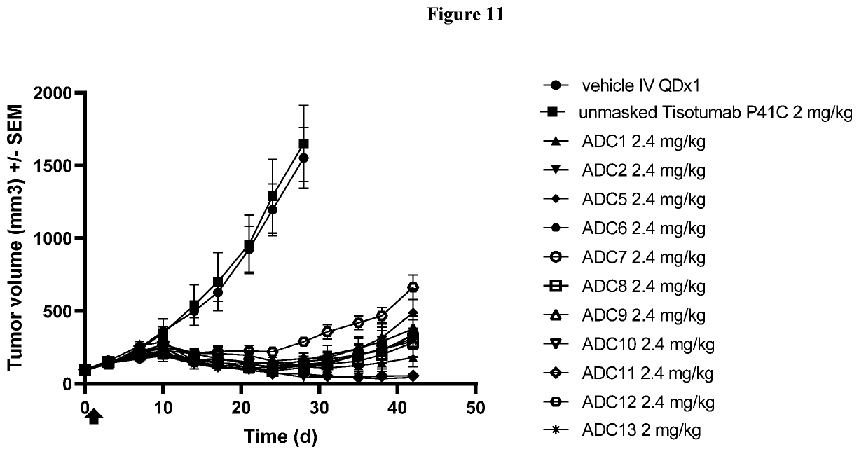

Results are presented in FIG. 11; every data point represents the average value of the absolute tumor volumes (mm³) for all mice in each group.

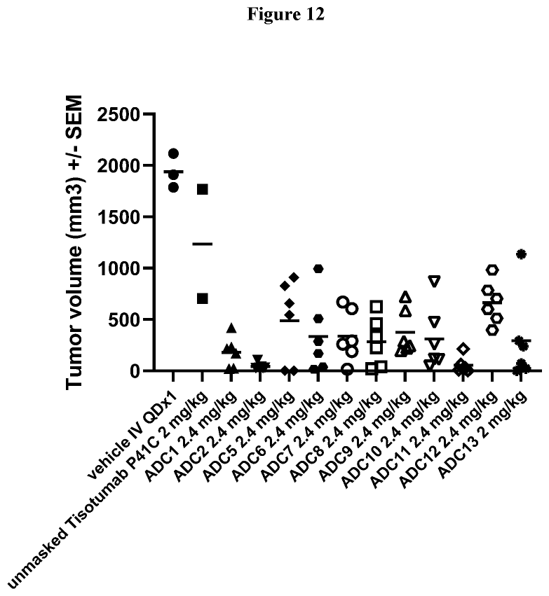

In FIG. 12 the absolute tumor volume for individual mice in each group on the last day (day 42) of the experiment is reflected. Line indicates the mean.

### Example 14: Use of Alternative Nb/Peptide Tag in Masking Moieties

Masking moieties were designed based on the “ALFA tag” as described in WO2020053239.

For the design of masking moieties a mutant with lower affinity for the ALFA tag peptide was chosen. The sequence of this mutant, Nb^{PE}, is disclosed as SEQ ID NO: 134 in WO2020053239 (SEQ ID NO: 91). The 2^{nd} masking moiety was based on a peptide sequence comprising the ALFA tag (SEQ ID NO: 92).

Based on the use of these sequences in masking moieties, anti-HER2 antibody trastuzumab was masked, whereby the peptide tag was linked to the N-terminus of the HC using a “Qg” spacer sequence and a linker containing the PLGLAG cleavage site, while the Nb^{PE} was linked to the LC with a linker containing the same cleavage site. A set of linkers (L12, L9, L7, L34 and L5) was used in all possible combinations, resulting in a set of 25 masked antibodies. The linkers used are reflected in Table 23.

TABLE 23
Linkers used for ALFA tag based MM

| | | SEQ |
|---|---|---|
| | Linker | ID |
| Linker (-Ab) | number | NO: |
| GGGGSGGGGSPLGLAGGGS-Ab | L12 | 93 |
| GGGGSPLGLAGGGS-Ab | L9 | 11 |
| GGSPLGLAGGGS-Ab | L7 | 19 |
| PLGLAGGGS-Ab | L34 | 94 |
| PLGLAG-Ab | L5 | 13 |

Masked antibodies were produced and purified. After purification the expression levels and the percentage of HMW complexes were determined as described in Example 1. The expression levels for all produced masked antibodies were above 100 μg/mL, and for the majority of antibodies above 200 μg/mL. HMW (%) were all very low (˜1%) for all linker combinations tested. Expression levels are reflected in Table 24, while HMW (%) is reflected in Table 25.

TABLE 24
Expression levels (μg/mL) for 25 masked trastuzumab
antibodies, where the mask was based on the
ALFA tag (Nb^{PE} and ALFA tag peptide (tag^{ALFA})

| Expression | | Qg- | Qg- | Qg- | Qg- |
|---|---|---|---|---|---|
| level | Qg-tag^{ALFA}- | tag^{ALFA}- | tag^{ALFA}- | tag^{ALFA}- | tag^{ALFA}- |
| (μg/mL) | L12-HC | L9-HC | L7-HC | L34-HC | L5-HC |
| Nb^{PE}-L12-LC | 215 | 214 | 209 | 177 | 215 |
| Nb^{PE}-L9-LC | 206 | 186 | 192 | 154 | 232 |
| Nb^{PE}-L7-LC | 227 | 200 | 209 | 164 | 223 |
| Nb^{PE}-L34-LC | 224 | 204 | 212 | 170 | 215 |
| Nb^{PE}-L5-LC | 229 | 192 | 173 | 148 | 206 |

TABLE 25
HMW (%) complexes for 25 masked trastuzumab
antibodies, where the mask was based on the
ALFA tag (Nb^{PE}) and ALFA tag peptide (tag^{ALFA})

| | Qg- | Qg- | Qg- | Qg- | Qg- |
|---|---|---|---|---|---|
| | tag^{ALFA}- | tag^{ALFA}- | tag^{ALFA}- | tag^{ALFA}- | tag^{ALFA}- |
| HMW (%) | L12-HC | L9-HC | L7-HC | L34-HC | L5-HC |
| Nb^{PE}-L12-LC | 1.2 | 1.2 | 1.3 | 1.2 | 1.0 |
| Nb^{PE}-L9-LC | 1.0 | 0.9 | 1.2 | 1.2 | 1.0 |
| Nb^{PE}-L7-LC | 1.0 | 0.9 | 1.0 | 1.1 | 0.9 |
| Nb^{PE}-L34-LC | 1.0 | 1.0 | 1.0 | 1.2 | 1.2 |
| Nb^{PE}-L5-LC | 0.7 | 0.8 | 0.9 | 1.2 | 1.2 |

Using the BLI method essentially as described in Example 4, binding of the masked PG-1T antibodies to immobilized HER2 protein was measured. BLI measurements indicated effective shielding could be reached by masking the trastuzumab antibody with masking moieties based on the ALFA tag (Nb^{PE}) and ALFA tag peptide (tag^{ALFA}).

Results of BLI binding for all 25 antibodies are reflected in Table 25. Although all antibodies were efficiently masked (binding to HER-2 was significantly reduced), it seemed that masking was most efficient with shorter linkers (L5, L34 and L7). For comparison; The BLI value obtained for unmasked (naked) trastuzumab was 331.2.

TABLE 25
Results of BLI binding for 25 masked trastuzumab
antibodies, where the mask was based on the
ALFA tag (Nb^{PE}) and ALFA tag peptide (tag^{ALFA})

| | Qg- | Qg- | Qg- | Qg- | Qg- |
|---|---|---|---|---|---|
| BLI binding | tag^{ALFA}- | tag^{ALFA}- | tag^{ALFA}- | tag^{ALFA}- | tag^{ALFA}- |
| results | L12-HC | L9-HC | L7-HC | L34-HC | L5-HC |
| Nb^{PE}-L12-LC | 5.32 | 5.03 | 4.75 | 5.01 | 4.81 |
| Nb^{PE}-L9-LC | 3.97 | 3.84 | 3.35 | 3.23 | 2.86 |
| Nb^{PE}-L7-LC | 3.41 | 2.86 | 2.49 | 2.21 | 1.67 |
| Nb^{PE}-L34-LC | 3.23 | 2.47 | 2.34 | 1.96 | 1.50 |
| Nb^{PE}-L5-LC | 1.13 | 0.63 | 0.47 | 0.32 | 0.34 |

SEQUENCES IN SEQUENCE LISTING

| SEQ | | |
|---|---|---|
| ID NO: | Description | Amino Acid Sequence |
| 1 | BC2T peptide | PDRKAAVSHWQQ |
| 2 | BC2T SA mutant | PDRKAAVAHWQQ |
| 3 | BC2T ST mutant | PDRKAAVTHWQQ |
| 4 | BC2-Nb (CDRs underlined) | QVQLVESGGGLVQPGGSLTLSCTASGFTLD |
| | | HYDIGWFRQAPGKEREGVSCINNSDDDTY |
| | | YADSVKGRFTIFMNNAKDTVYLQMNSLKP |
| | | EDTAIYYCAEARGCKRGRYEYDFWGQGTQ |
| | | VTVSS |
| 5 | BC2-Nb CDR3 | ARGCKRGRYEYDFW |
| 6 | CDR3 BC2_{Nb}-R106A (RA) | ARGCKRGAYEYDFW |
| 7 | CDR3 BC2_{Nb}-R106E (RE) | ARGCKRGEYEYDFW |
| 8 | CDR3 BC2_{Nb}-R106S (RS) | ARGCKRGSYEYDFW |
| 9 | Qg spacer | QGQSGQG |
| 10 | Qv spacer | QVQLVES |
| 11 | L9 MMP2 cleavable linker | GGGGSPLGLAGGGS |
| 12 | L13 scrambled linker | GGGGSLALGPGGGS |
| 13 | MMP cleavage site/L5 | PLGLAG |
| 14 | Matriptase/uPA cleavage | LSGRSDNH |
| | site/L14 | |
| 15 | MMP14 cleavage site | ISSGLL |
| 16 | “2001” cleavage site | ISSGLLSGRSDNH |
| 17 | MMP14/uPA combination | ISSGLLSSGGSGGSLSGRSDNH |
| | cleavage sequence | |
| 18 | uPA/MMP14 combination | LSGRSDNHGGSGGSISSGLLSS |
| | Cleavage sequence | |
| 19 | L7 | GGSPLGLAGGGS |
| 20 | Spacer sequence | GGGGS |
| 21 | HC Anti-HER2: | EVQLVESGGGLVQPGGSLRLSCAASGFNIK |
| | trastuzumab | DTYIHWVRQAPGKGLEWVARIYPTNGYTR |
| | | YADSVKGRFTISADTSKNTAYLQMNSLRAE |
| | | DTAVYYCSRWGGDGFYAMDYWGQGTLVT |
| | | VSSASTKGPSVFPLAPSSKSTSGGTAALGC |
| | | LVKDYFPEPVTVSWNSGALTSGVHTFPAVL |
| | | QSSGLYSLSSVVTVPSSSLGTQTYICNVNHK |
| | | PSNTKVDKKVEPKSCDKTHTCPPCPAPELL |
| | | GGPSVFLFPPKPKDTLMISRTPEVTCVVVD |
| | | VSHEDPEVKFNWYVDGVEVHNAKTKPREE |
| | | QYNSTYRVVSVLTVLHQDWLNGKEYKCK |
| | | VSNKALPAPIEKTISKAKGQPREPQVYTLPP |
| | | SREEMTKNQVSLTCLVKGFYPSDIAVEWES |
| | | NGQPENNYKTTPPVLDSDGSFFLYSKLTVD |
| | | KSRWQQGNVFSCSVMHEALHNHYTQKSLS |
| | | LSPG |
| 22 | HC (P41C) Anti-HER2: | EVQLVESGGGLVQPGGSLRLSCAASGFNIK |
| | trastuzumab | DTYIHWVRQACGKGLEWVARIYPTNGYTR |
| | | YADSVKGRFTISADTSKNTAYLQMNSLRAE |
| | | DTAVYYCSRWGGDGFYAMDYWGQGTLV |
| | | TVSSASTKGPSVFPLAPSSKSTSGGTAALGC |
| | | LVKDYFPEPVTVSWNSGALTSGVHTFPAVL |
| | | QSSGLYSLSSVVTVPSSSLGTQTYICNVNHK |
| | | PSNTKVDKKVEPKSCDKTHTCPPCPAPELL |
| | | GGPSVFLFPPKPKDTLMISRTPEVTCVVVD |
| | | VSHEDPEVKFNWYVDGVEVHNAKTKPREE |
| | | QYNSTYRVVSVLTVLHQDWLNGKEYKCK |
| | | VSNKALPAPIEKTISKAKGQPREPQVYTLPP |
| | | SREEMTKNQVSLTCLVKGFYPSDIAVEWES |
| | | NGQPENNYKTTPPVLDSDGSFFLYSKLTVD |
| | | KSRWQQGNVFSCSVMHEALHNHYTQKSLS |
| | | LSPG |
| 23 | LC Anti-HER2: | DIQMTQSPSSLSASVGDRVTITCRASQDVN |
| | trastuzumab | TAVAWYQQKPGKAPKLLIYSASFLYSGVPS |
| | | RFSGSRSGTDFTLTISSLQPEDFATYYCQQH |
| | | YTTPPTFGQGTKVEIKRTVAAPSVFIFPPSD |
| | | EQLKSGTASVVCLLNNFYPREAKVQWKVDN |
| | | ALQSGNSQESVTEQDSKDSTYSLSSTLTLSK |
| | | ADYEKHKVYACEVTHQGLSSPVTKSFNRG |
| | | EC |
| 24 | HC Anti-EpCAM: ING-1 | QIQLVQSGPEVKKPGESVKISCKASGYTFT |
| | | KYGMNWVKQAPGQGLKWMGWINTYTEEPTY |
| | | GDDFKGRFTFTLDTSTSTAYLEISSLRSED |
| | | TATYFCARFGSAVDYWGQGTLVTVSSAST |
| | | KGPSVFPLAPSSKSTSGGTAALGCLVKDYF |
| | | PEPVTVSWNSGALTSGVHTFPAVLQSSGLY |
| | | SLSSVVTVPSSSLGTQTYICNVNHKPSNTKV |
| | | DKRVEPKSCDKTHTCPPCPAPELLGGPSVF |
| | | LFPPKPKDTLMISRTPEVTCVVVDVSHEDP |
| | | EVKFNWYVDGVEVHNAKTKPREEQYNSTY |
| | | RVVSVLTVLHQDWLNGKEYKCKVSNKALP |
| | | APIEKTISKAKGQPREPQVYTLPPSRDELT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNHYTQKSLSLSPGK |
| 25 | HC (P41C) Anti-EpCAM: | QIQLVQSGPEVKKPGESVKISCKASGYTFTK |
| | ING-1 | YGMNWVKQACGQGLKWMGWINTYTEEP |
| | | TYGDDFKGRFTFTLDTSTSTAYLEISSLRSE |
| | | DTATYFCARFGSAVDYWGQGTLVTVSSAS |
| | | TKGPSVFPLAPSSKSTSGGTAALGCLVKDY |
| | | FPEPVTVSWNSGALTSGVHTFPAVLQSSGL |
| | | YSLSSVVTVPSSSLGTQTYICNVNHKPSNTK |
| | | VDKRVEPKSCDKTHTCPPCPAPELLGGPSV |
| | | FLFPPKPKDTLMISRTPEVTCVVVDVSHEDP |
| | | EVKFNWYVDGVEVHNAKTKPREEQYNST |
| | | YRVVSVLTVLHQDWLNGKEYKCKVSNKA |
| | | LPAPIEKTISKAKGQPREPQVYTLPPSRDEL |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPGK |
| 26 | LC Anti-EpCAM: ING-1 | DIVMTQSALSNPVTLGESGSISCRSSKSLLH |
| | | SNGITYLYWYLQKPGQSPQLLIYQMSNRAS |
| | | GVPDRFSSSGSGTDFTLKISRVEAEDVGVY |
| | | YCAQNLELPRTFGQGTKLEMKRTVAAPSV |
| | | FIFPPSDEQLKSGTASVVCLLNNFYPREAKV |
| | | QWKVDNALQSGNSQESVTEQDSKDSTYSL |
| | | SSTLTLSKADYEKHKVYACEVTHQGLSSPV |
| | | TKSFNRGEC |
| 27 | HC Anti-EpCAM: | EVQLLESGGGVVQPGRSLRLSCAASGFTFS |
| | adecatumumab | SYGMHWVRQAPGKGLEWVAVISYDGSNK |
| | | YYADSVKGRFTISRDNSKNTLYLQMNSLR |
| | | AEDTAVYYCAKDMGWGSGWRPYYYYGM |
| | | DVWGQGTTVTVSSASTKGPSVFPLAPSSKS |
| | | TSGGTAALGCLVKDYFPEPVTVSWNSGAL |
| | | TSGVHTFPAVLQSSGLYSLSSVVTVPSSSLG |
| | | TQTYICNVNHKPSNTKVDKKVEPKSCDKT |
| | | HTCPPCPAPELLGGPSVFLFPPKPKDTLMIS |
| | | RTPEVTCVVVDVSHEDPEVKFNWYVDGVE |
| | | VHNAKTKPREEQYNSTYRVVSVLTVLHQD |
| | | WLNGKEYKCKVSNKALPAPIEKTISKAKGQ |
| | | PREPQVYTLPPSRDELTKNQVSLTCLVKGF |
| | | YPSDIAVEWESNGQPENNYKTTPPVLDSDG |
| | | SFFLYSKLTVDKSRWQQGNVFSCSVMHEA |
| | | LHNHYTQKSLSLSPGK |
| 28 | HC (P41C) Anti-EpCAM: | EVQLLESGGGVVQPGRSLRLSCAASGFTFS |
| | adecatumumab | SYGMHWVRQACGKGLEWVAVISYDGSNK |
| | | YYADSVKGRFTISRDNSKNTLYLQMNSLR |
| | | AEDTAVYYCAKDMGWGSGWRPYYYYGM |
| | | DVWGQGTTVTVSSASTKGPSVFPLAPSSKS |
| | | TSGGTAALGCLVKDYFPEPVTVSWNSGAL |
| | | TSGVHTFPAVLQSSGLYSLSSVVTVPSSSLG |
| | | TQTYICNVNHKPSNTKVDKKVEPKSCDKT |
| | | HTCPPCPAPELLGGPSVFLFPPKPKDTLMIS |
| | | RTPEVTCVVVDVSHEDPEVKFNWYVDGVE |
| | | VHNAKTKPREEQYNSTYRVVSVLTVLHQD |
| | | WLNGKEYKCKVSNKALPAPIEKTISKAKGQ |
| | | PREPQVYTLPPSRDELTKNQVSLTCLVKGF |
| | | YPSDIAVEWESNGQPENNYKTTPPVLDSDG |
| | | SFFLYSKLTVDKSRWQQGNVFSCSVMHEA |
| | | LHNHYTQKSLSLSPGK |
| 29 | LC Anti-EpCAM: | ELQMTQSPSSLSASVGDRVTITCRTSQSISS |
| | adecatumumab | YLNWYQQKPGQPPKLLIYWASTRESGVPD |
| | | RFSGSGSGTDFTLTISSLQPEDSATYYCQQS |
| | | YDIPYTFGQGTKLEIKRTVAAPSVFIFPPSDE |
| | | QLKSGTASVVCLLNNFYPREAKVQWKVDN |
| | | ALQSGNSQESVTEQDSKDSTYSLSSTLTLSK |
| | | ADYEKHKVYACEVTHQGLSSPVTKSFNRG |
| | | EC |
| 30 | HC Anti- | QVQLQQSGSELKKPGASVKVSCKASGYTF |
| | TROP2: | TNYGMNWVKQAPGQGLKWMGWINTYTG |
| | sacituzumab hRS7 | EPTYTDDFKGRFAFSLDTSVSTAYLQISSLK |
| | | ADDTAVYFCARGGFGSSYWYFDVWGQGS |
| | | LVTVSSASTKGPSVFPLAPSSKSTSGGTAAL |
| | | GCLVKDYFPEPVTVSWNSGALTSGVHTFPA |
| | | VLQSSGLYSLSSVVTVPSSSLGTQTYICNVN |
| | | HKPSNTKVDKKVEPKSCDKTHTCPPCPAPE |
| | | LLGGPSVFLFPPKPKDTLMISRTPEVTCVVV |
| | | DVSHEDPEVKFNWYVDGVEVHNAKTKPR |
| | | EEQYNSTYRVVSVLTVLHQDWLNGKEYKC |
| | | KVSNKALPAPIEKTISKAKGQPREPQVYTLP |
| | | PSRDELTKNQVSLTCLVKGFYPSDIAVEWE |
| | | SNGQPENNYKTTPPVLDSDGSFFLYSKLTV |
| | | DKSRWQQGNVFSCSVMHEALHNHYTQKS |
| | | LSLSPGK |
| 31 | HC (P(41C) Anti- | QVQLQQSGSELKKPGASVKVSCKASGYTF |
| | TROP2: | TNYGMNWVKQACGQGLKWMGWINTYTG |
| | sacituzumab hRS7 | EPTYTDDFKGRFAFSLDTSVSTAYLQISSLK |
| | | ADDTAVYFCARGGFGSSYWYFDVWGQGS |
| | | LVTVSSASTKGPSVFPLAPSSKSTSGGTAAL |
| | | GCLVKDYFPEPVTVSWNSGALTSGVHTFPA |
| | | VLQSSGLYSLSSVVTVPSSSLGTQTYICNVN |
| | | HKPSNTKVDKKVEPKSCDKTHTCPPCPAPE |
| | | LLGGPSVFLFPPKPKDTLMISRTPEVTCVVV |
| | | DVSHEDPEVKFNWYVDGVEVHNAKTKPR |
| | | EEQYNSTYRVVSVLTVLHQDWLNGKEYKC |
| | | KVSNKALPAPIEKTISKAKGQPREPQVYTLP |
| | | PSRDELTKNQVSLTCLVKGFYPSDIAVEWE |
| | | SNGQPENNYKTTPPVLDSDGSFFLYSKLTV |
| | | DKSRWQQGNVFSCSVMHEALHNHYTQKS |
| | | LSLSPGK |
| 32 | LC Anti- | DIQLTQSPSSLSASVGDRVSITCKASQDVSI |
| | TROP2: | AVAWYQQKPGKAPKLLIYSASYRYTGVPD |
| | sacituzumab hRS7 | RFSGSGSGTDFTLTISSLQPEDFAVYYCQQH |
| | | YITPLTFGAGTKVEIKRTVAAPSVFIFPPSDE |
| | | QLKSGTASVVCLLNNFYPREAKVQWKVDN |
| | | ALQSGNSQESVTEQDSKDSTYSLSSTLTLSK |
| | | ADYEKHKVYACEVTHQGLSSPVTKSFNRG |
| | | EC |
| 33 | HC Anti-TF: MORAb-066, | QVQLVESGGGVVQPGRSLRLSCKASGFNIK |
| | CNT0860 | DYYMHWVRQAPGKGLEWIGLIDPENGNTI |
| | | YDPKFQGRFTISADNSKNTLFLQMDSLRPE |
| | | DTAVYYCARDNSYYFDYWGQGTPVTVSS |
| | | ASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPSN |
| | | TKVDKKVEPKSCDKTHTCPPCPAPELLGGP |
| | | SVFLFPPKPKDTLMISRTPEVTCVVVDVSHE |
| | | DPEVKFNWYVDGVEVHNAKTKPREEQYN |
| | | STYRVVSVLTVLHQDWLNGKEYKCKVSN |
| | | KALPAPIEKTISKAKGQPREPQVYTLPPSRD |
| | | ELTKNQVSLTCLVKGFYPSDIAVEWESNGQ |
| | | PENNYKTTPPVLDSDGSFFLYSKLTVDKSR |
| | | WQQGNVFSCSVMHEALHNHYTQKSLSLSP |
| | | GK |
| 34 | HC (P41C) Anti-TF: | QVQLVESGGGVVQPGRSLRLSCKASGFNIK |
| | MORAb-066 | DYYMHWVRQACGKGLEWIGLIDPENGNTI |
| | | YDPKFQGRFTISADNSKNTLFLQMDSLRPE |
| | | DTAVYYCARDNSYYFDYWGQGTPVTVSS |
| | | ASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPSN |
| | | TKVDKKVEPKSCDKTHTCPPCPAPELLGGP |
| | | SVFLFPPKPKDTLMISRTPEVTCVVVDVSHE |
| | | DPEVKFNWYVDGVEVHNAKTKPREEQYN |
| | | STYRVVSVLTVLHQDWLNGKEYKCKVSN |
| | | KALPAPIEKTISKAKGQPREPQVYTLPPSRD |
| | | ELTKNQVSLTCLVKGFYPSDIAVEWESNGQ |
| | | PENNYKTTPPVLDSDGSFFLYSKLTVDKSR |
| | | WQQGNVFSCSVMHEALHNHYTQKSLSLSP |
| | | GK |
| 35 | LC Anti-TF: MORAb-066 | DIQMTQSPSSLSASVGDRVTITCKASQDIRK |
| | | YLNWYQQKPGKAPKLLIYYATSLADGVPS |
| | | RFSGSGSGTDYTFTISSLQPEDIATYYCLQH |
| | | GESPYTFGQGTKLEITRTVAAPSVFIFPPSDE |
| | | QLKSGTASVVCLLNNFYPREAKVQWKVDN |
| | | ALQSGNSQESVTEQDSKDSTYSLSSTLTLSK |
| | | ADYEKHKVYACEVTHQGLSSPVTKSFNRG |
| | | EC |
| 36 | HC Anti-TF: tisotumab (TF- | EVQLLESGGGLVQPGGSLRLSCAASGFTFS |
| | 011) | NYAMSWVRQAPGKGLEWVSSISGSGDYTY |
| | | YTDSVKGRFTISRDNSKNTLYLQMNSLRAE |
| | | DTAVYYCARSPWGYYLDSWGQGTLVTVS |
| | | SASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPSN |
| | | TKVDKRVEPKSCDKTHTCPPCPAPELLGGP |
| | | SVFLFPPKPKDTLMISRTPEVTCVVVDVSHE |
| | | DPEVKFNWYVDGVEVHNAKTKPREEQYN |
| | | STYRVVSVLTVLHQDWLNGKEYKCKVSN |
| | | KALPAPIEKTISKAKGQPREPQVYTLPPSRE |
| | | EMTKNQVSLTCLVKGFYPSDIAVEWESNG |
| | | QPENNYKTTPPVLDSDGSFFLYSKLTVDKS |
| | | RWQQGNVFSCSVMHEALHNHYTQKSLSLS |
| | | PGK |
| 37 | HC (P41C) Anti-TF: | EVQLLESGGGLVQPGGSLRLSCAASGFTFS |
| | tisotumab (TF-011) | NYAMSWVRQACGKGLEWVSSISGSGDYT |
| | | YYTDSVKGRFTISRDNSKNTLYLQMNSLRA |
| | | EDTAVYYCARSPWGYYLDSWGQGTLVTV |
| | | SSASTKGPSVFPLAPSSKSTSGGTAALGCLV |
| | | KDYFPEPVTVSWNSGALTSGVHTFPAVLQS |
| | | SGLYSLSSVVTVPSSSLGTQTYICNVNHKPS |
| | | NTKVDKRVEPKSCDKTHTCPPCPAPELLGG |
| | | PSVFLFPPKPKDTLMISRTPEVTCVVVDVSH |
| | | EDPEVKFNWYVDGVEVHNAKTKPREEQY |
| | | NSTYRVVSVLTVLHQDWLNGKEYKCKVS |
| | | NKALPAPIEKTISKAKGQPREPQVYTLPPSR |
| | | EEMTKNQVSLTCLVKGFYPSDIAVEWESN |
| | | GQPENNYKTTPPVLDSDGSFFLYSKLTVDK |
| | | SRWQQGNVFSCSVMHEALHNHYTQKSLSL |
| | | SPGK |
| 38 | LC Anti-TF: tisotumab | DIQMTQSPPSLSASAGDRVTITCRASQGISS |
| | (TF-011) | RLAWYQQKPEKAPKSLIYAASSLQSGVPSR |
| | | FSGSGSGTDFTLTISSLQPEDFATYYCQQYN |
| | | SYPYTFGQGTKLEIKRTVAAPSVFIFPPSDE |
| | | QLKSGTASVVCLLNNFYPREAKVQWKVDN |
| | | ALQSGNSQESVTEQDSKDSTYSLSSTLTLSK |
| | | ADYEKHKVYACEVTHQGLSSPVTKSFNRG |
| | | EC |
| 39 | HC Anti-CD137 (4-1BB): | QVQLQQWGAGLLKPSETLSLTCAVYGGSF |
| | urelumab | SGYYWSWIRQSPEKGLEWIGEINHGGYVTY |
| | | NPSLESRVTISVDTSKNQFSLKLSSVTAADT |
| | | AVYYCARDYGPGNYDWYFDLWGRGTLVT |
| | | VSSASTKGPSVFPLAPCSRSTSESTAALGCL |
| | | VKDYFPEPVTVSWNSGALTSGVHTFPAVL |
| | | QSSGLYSLSSVVTVPSSSLGTKTYTCNVDH |
| | | KPSNTKVDKRVESKYGPPCPPCPAPEFLGG |
| | | PSVFLFPPKPKDTLMISRTPEVTCVVVDVSQ |
| | | EDPEVQFNWYVDGVEVHNAKTKPREEQFN |
| | | STYRVVSVLTVLHQDWLNGKEYKCKVSN |
| | | KGLPSSIEKTISKAKGQPREPQVYTLPPSQE |
| | | EMTKNQVSLTCLVKGFYPSDIAVEWESNG |
| | | QPENNYKTTPPVLDSDGSFFLYSRLTVDKS |
| | | RWQEGNVFSCSVMHEALHNHYTQKSLSLS |
| | | LGK |
| 40 | LC Anti-CD137 (4-1BB): | EIVLTQSPATLSLSPGERATLSCRASQSVSS |
| | urelumab | YLAWYQQKPGQAPRLLIYDASNRATGIPAR |
| | | FSGSGSGTDFTLTISSLEPEDFAVYYCQQRS |
| | | NWPPALTFGGGTKVEIKRTVAAPSVFIFPPS |
| | | DEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSF |
| | | NRGEC |
| 41 | HC Anti-CTLA4: | QVQLVESGGGVVQPGRSLRLSCAASGFTFS |
| | tremelimumab | SYGMHWVRQAPGKGLEWVAVIWYDGSN |
| | | KYYADSVKGRFTISRDNSKNTLYLQMNSL |
| | | RAEDTAVYYCARDPRGATLYYYYYGMDV |
| | | WGQGTTVTVSSASTKGPSVFPLAPCSRSTS |
| | | ESTAALGCLVKDYFPEPVTVSWNSGALTSG |
| | | VHTFPAVLQSSGLYSLSSVVTVPSSNFGTQT |
| | | YTCNVDHKPSNTKVDKTVERKCCVECPPC |
| | | PAPPVAGPSVFLFPPKPKDTLMISRTPEVTC |
| | | VVVDVSHEDPEVQFNWYVDGVEVHNAKT |
| | | KPREEQFNSTFRVVSVLTVVHQDWLNGKE |
| | | YKCKVSNKGLPAPIEKTISKTKGQPREPQV |
| | | YTLPPSREEMTKNQVSLTCLVKGFYPSDIA |
| | | VEWESNGQPENNYKTTPPMLDSDGSFFLYS |
| | | KLTVDKSRWQQGNVFSCSVMHEALHNHY |
| | | TQKSLSLSPGK |
| 42 | LC Anti-CTLA4: | DIQMTQSPSSLSASVGDRVTITCRASQSINS |
| | tremelimumab | YLDWYQQKPGKAPKLLIYAASSLQSGVPSR |
| | | FSGSGSGTDFTLTISSLQPEDFATYYCQQYY |
| | | STPFTFGPGTKVEIKRTVAAPSVFIFPPSDEQ |
| | | LKSGTASVVCLLNNFYPREAKVQWKVDNA |
| | | LQSGNSQESVTEQDSKDSTYSLSSTLTLSKA |
| | | DYEKHKVYACEVTHQGLSSPVTKSFNRGE |
| | | C |
| 43 | HAVT20 leader sequence | MACPGFLWALVISTCLEFSMA |
| 44 | Spot-Tag® | PDRVRAVSHWSS |
| 45 | C-tag | EPEA |
| 46 | ALFA-tag | SRLEEELRRRLTE |
| 47 | ALFA-tag Nb | EVQLQESGGGLVQPGGSLRLSCTASGVTIS |
| | (Nb ALFA) | ALNAMAMGWYRQAPGERRVMVAAVSER |
| | | GNAMYRESVQGRFTVTRDFTNKMVSLQM |
| | | DNLKPEDTAVYYCHVLEDRVDSFHDYWG |
| | | QGTQVTVSS |
| 48 | BC2T peptide variant | PVRSAALSQWSS |
| 49 | BC2T peptide variant | PDRVRAVSHWSS |
| 50 | BC2T peptide variant | ADRVRAVSHWSS |
| 51 | BCT2 peptide formula | X₁X₂RX₄X₅AX₇X₈X₉WX₁₁X₁₂, |
| | | wherein X₁ can be P or A, |
| | | wherein X₂ can be D or a conservative |
| | | substitution of D, |
| | | wherein X₄ can be K, or a conservative |
| | | substitution of K, or S, |
| | | wherein X₅ can be A or R, or a conservative |
| | | substitution of A or R, |
| | | wherein X₇ can be V or a conservative |
| | | substitution of V, |
| | | wherein X₈ can be S, A, V or T |
| | | wherein X₉ can be H or a conservative |
| | | substitution of H, |
| | | wherein X₁₁ and X₁₂ can each independently be |
| | | Q or a conservative substitution of Q |
| 52 | Matriptase cleavage site | IPVSLR |
| 53 | Matriptase cleavage site | RQARVVNG |
| 54 | Matriptase cleavage site | PMAKK |
| 55 | L15 | GGLSGRSDNH |
| 56 | L16 | SGGLSGRSDNH |
| 57 | L17 | GSGGLSGRSDNH |
| 58 | L18 | GGLSGRSDNHGGS |
| 59 | L19 | SGGLSGRSDNHGGS |
| 60 | L20 | GSGGLSGRSDNHGGS |
| 61 | L21 | GGSGGLSGRSDNHGGS |
| 62 | L22 | GGGSGGLSGRSDNHGGS |
| 63 | L23 | GGGGSGGLSGRSDNHGGS |
| 64 | L24 | GGGGSGGSGGLSGRSDNHGGS |
| 65 | L25 | GGGSSGGSGGSGGLSGRSDNHGGS |
| 66 | L26 | GGGSGGSPLGLAGGGS |
| 67 | L27 | GGSGGSPLGLAGGGS |
| 68 | L29 | GGGSPLGLAGGGS |
| 69 | L30 | GGPLGLAGGGS |
| 70 | MMP2 cleavage site | PLGVR |
| 71 | SYD1030 (anti PSMA) HC | ASTKGPSVFPLAPSSKSTSGGTAALGCLVKDYF |
| | 41C | PEPVTVSWNSGALTSGVHTFPAVLQSSGLYSLS |
| | | SVVTVPSSSLGTQTYICNVNHKPSNTKVDKKVE |
| | | PKSCDKTHTCPPCPAPELLGGPSVFLFPPKPKDT |
| | | LMISRTPEVTCVVVDVSHEDPEVKFNWYVDGV |
| | | EVHNAKTKPREEQYNSTYRVVSVLTVLHQDW |
| | | LNGKEYKCKVSNKALPAPIEKTISKAKGQPREP |
| | | QVYTLPPSRDELTKNQVSLTCLVKGFYPSDIAV |
| | | EWESNGQPENNYKTTPPVLDSDGSFFLYSKLTV |
| | | DKSRWQQGNVFSCSVMHEALHNHYTQKSLSLS |
| | | PGK |
| 72 | SYD1030 (anti PSMA) LC | DIVMTQSPSSLSASVGDRVTITCKASQDVGTAV |
| | | DWYQQKPGKAPKLLIYWASTRHTGVPDRFTGS |
| | | GSGTDFTLTISSLQPEDFADYFCQQYNSYPLTFG |
| | | GGTKLEIK |
| 73 | Rituximab HC 41C | QVQLQQPGAELVKPGASVKMSCKASGYTFTSY |
| | | NMHWVKQTCGRGLEWIGAIYPGNGDTSYNQK |
| | | FKGKATLTADKSSSTAYMQLSSLTSEDSAVYY |
| | | CARSTYYGGDWYFNVWGAGTTVTVSAASTKG |
| | | PSVFPLAPSSKSTSGGTAALGCLVKDYFPEPVT |
| | | VSWNSGALTSGVHTFPAVLQSSGLYSLSSVVTV |
| | | PSSSLGTQTYICNVNHKPSNTKVDKKAEPKSCD |
| | | KTHTCPPCPAPELLGGPSVFLFPPKPKDTLMISR |
| | | TPEVTCVVVDVSHEDPEVKFNWYVDGVEVHN |
| | | AKTKPREEQYNSTYRVVSVLTVLHQDWLNGK |
| | | EYKCKVSNKALPAPIEKTISKAKGQPREPQVYT |
| | | LPPSRDELTKNQVSLTCLVKGFYPSDIAVEWES |
| | | NGQPENNYKTTPPVLDSDGSFFLYSKLTVDKSR |
| | | WQQGNVFSCSVMHEALHNHYTQKSLSLSPGK |
| 74 | Rituximab LC | QIVLSQSPAILSASPGEKVTMTCRASSSVSYIHW |
| | | FQQKPGSSPKPWIYATSNLASGVPVRFSGSGSG |
| | | TSYSLTISRVEAEDAATYYCQQWTSNPPTFGGG |
| | | TKLEIKRTVAAPSVFIFPPSDEQLKSGTASVVCL |
| | | LNNFYPREAKVQWKVDNALQSGNSQESVTEQ |
| | | DSKDSTYSLSSTLTLSKADYEKHKVYACEVTH |
| | | QGLSSPVTKSFNRGEC |
| 75 | MMP2 site | IPVSLRSG |
| 76 | BC2_{Nb}-R106A (RA) | QVQLVESGGGLVQPGGSLTLSCTASGFTLDHY |
| | | DIGWFRQAPGKEREGVSCINNSDDDTYYADSV |
| | | KGRFTIFMNNAKDTVYLQMNSLKPEDTAIYYC |
| | | AEARGCKRGAYEYDFWGQGTQVTVSS |
| 77 | BC2_{Nb}-R106E (RE) | QVQLVESGGGLVQPGGSLTLSCTASGFTLDHY |
| | | DIGWFRQAPGKEREGVSCINNSDDDTYYADSV |
| | | KGRFTIFMNNAKDTVYLQMNSLKPEDTAIYYC |
| | | AEARGCKRGEYEYDFWGQGTQVTVSS |
| 78 | BC2_{Nb}-R106S (RS) | QVQLVESGGGLVQPGGSLTLSCTASGFTLDHY |
| | | DIGWFRQAPGKEREGVSCINNSDDDTYYADSV |
| | | KGRFTIFMNNAKDTVYLQMNSLKPEDTAIYYC |
| | | AEARGCKRGSYEYDFWGQGTQVTVSS |
| 79 | BC2_{Nb humanized 1} | QVQLVESGGGLVQPGGSLTLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGRYEYDFWGQGTQVTVSS |
| 80 | BC2_{Nb humanized 2} | QVQLVESGGGLVQPGGSLRLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGRYEYDFWGQGTQVTVSS |
| 81 | BC2_{Nb humanized 3} | QVQLVESGGGSVQPGGSLRLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGRYEYDFWGQGTQVTVSS |
| 82 | BC2_{Nb humanized 1}-R106A (RAₕ₁) | QVQLVESGGGLVQPGGSLTLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGAYEYDFWGQGTQVTVSS |
| 83 | BC2_{Nb humanized 2}-R106A (RAₕ₂) | QVQLVESGGGLVQPGGSLRLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGAYEYDFWGQGTQVTVSS |
| 84 | BC2_{Nb humanized 3}-R106A (RAₕ₃) | QVQLVESGGGSVQPGGSLRLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGAYEYDFWGQGTQVTVSS |
| 85 | BC2_{Nb humanized 1}-R106E (REₕ₁) | QVQLVESGGGLVQPGGSLTLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGEYEYDFWGQGTQVTVSS |
| 86 | BC2_{Nb humanized 2}-R106E (REₕ₂) | QVQLVESGGGLVQPGGSLRLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGEYEYDFWGQGTQVTVSS |
| 87 | BC2_{Nb humanized 3}-R106E (REₕ₃) | QVQLVESGGGSVQPGGSLRLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGEYEYDFWGQGTQVTVSS |
| 88 | BC2_{Nb humanized 1}-R106S (RSₕ₁) | QVQLVESGGGLVQPGGSLTLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGSYEYDFWGQGTQVTVSS |
| 89 | BC2_{Nb humanized 2}-R106S (RSₕ₂) | QVQLVESGGGLVQPGGSLRLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGSYEYDFWGQGTQVTVSS |
| 90 | BC2_{Nb humanized 3}-R106S (RSₕ₃) | QVQLVESGGGSVQPGGSLRLSCTASGFTLDHY |
| | | DIGWFRQAPGKEREGVSCINNSDDDTYYADSV |
| | | KGRFTIFMNNAKDTVYLQMNSLRAEDTAIYYC |
| | | AEARGCKRGSYEYDFWGQGTQVTVSS |
| 91 | Nb^{PE} (PE mutant of ALFA tag | EVQLQESGGGLVQPGGSLRLSCTASGVTISAL |
| | Nb, SEQ ID NO: 134 in | NAMAMGWYRQAPGEERVMVAAVSSRGNAMYRE |
| | WO2020053239) | SVQGRFTVTRDFTNKMVSLQMDNLKPEDTAVY |
| | | YCHVLEDRVDSFHDYWGQGTQVTVSS |
| 92 | ALFA tag peptide used in MM | PSRLEEELRRRLTEP |
| 93 | L12 | GGGGSGGGGSPLGLAGGGS |
| 94 | L34 | PLGLAGGGS |
| 95 | BCT2 peptide formula (2) | X₁X₂X₃X₄X₅AX₇X₈X₉WX₁₁X₁₂ |
| | | wherein X₁ can be P or A, |
| | | wherein X₂ can be D or a conservative |
| | | substitution of D, |
| | | wherein X₃ can be R or K, |
| | | wherein X₄ can be K, or a conservative |
| | | substitution of K, or S, |
| | | wherein X₅ can be A or R, or a conservative |
| | | substitution of A or R, |
| | | wherein X₇ can be V or a conservative |
| | | substitution of V, |
| | | wherein X₈ can be S, A, V or T |
| | | wherein X₉ can be H or a conservative |
| | | substitution of H, |
| | | wherein X₁₁ and X₁₂ can each independently be |
| | | Q or a conservative substitution of Q |

1. An activatable antibody or antigen binding fragment thereof, comprising an antigen binding site, comprising a heavy chain variable domain and a light chain variable domain capable of binding to a target protein, wherein said antigen binding site is provided with a mask suitable for inhibiting binding of the antigen binding site to the target protein, the mask comprising two masking moieties:
a) a masking moiety linked to the N-terminus of the variable heavy chain domain, and
b) a masking moiety linked to the N-terminus of the variable light chain domain,
wherein at least one of the masking moieties is linked through a cleavable linker, and wherein one masking moiety comprises an antigenic peptide and the other masking moiety comprises a single domain antibody (sdAb) specifically binding to the antigenic peptide.

2. The activatable antibody or antigen binding fragment thereof according to claim 1, wherein all masking moieties are linked through a cleavable linker.

3. The activatable antibody or antigen binding fragment thereof according to claim 1, wherein the cleavable linker is a protease cleavable peptidic linker.

4. The activatable antibody or antigen binding fragment thereof according to claim 1, wherein the sdAb is a nanobody.

5. The activatable antibody or antigen binding fragment thereof according to claim 1, comprising two antigen binding sites, wherein both antigen binding sites are provided with a mask.

6. The activatable antibody or antigen binding fragment thereof according to claim 1, wherein the antigenic peptide is 4-20 amino acid residues in length.

7. The activatable antibody or antigen binding fragment thereof according to claim 1, wherein the antigenic peptide is 12-20 amino acid residues in length and comprises or consists of an amino acid sequence as defined in SEQ ID NO: 51.

8. The activatable antibody or antigen binding fragment thereof according to claim 1, wherein the antigenic peptide comprises an amino acid sequence as defined in SEQ ID NO: 1, SEQ ID NO: 2 (SA mutant) or SEQ ID NO: 3 (ST mutant).

9. The activatable antibody or antigen binding fragment thereof according to claim 4, wherein the nanobody comprises the amino acid sequence as defined in SEQ ID NO: 4, a humanized version thereof, or a variant of SEQ ID NO: 4, with the amino acid sequence defined in SEQ ID NO: 76 (RA mutant), SEQ ID NO: 77 (RE mutant), or SEQ ID NO: 78 (RS mutant), or a humanized version of said variant.

10. The activatable antibody or antigen binding fragment thereof according to claim 9, wherein the antigenic peptide comprises or consists of a peptide with the amino acid sequence defined in SEQ ID NO: 2 (SA mutant) and the nanobody comprises the amino acid sequence as defined in SEQ ID NO: 78 (RS mutant) or a humanized version thereof.

11. The activatable antibody or antigen binding fragment thereof according to claim 1, wherein the masking moiety comprising the sdAb is linked to the N-terminus of the light chain variable domain and the masking moiety comprising the antigenic peptide is linked to the N-terminus of the heavy chain variable domain.

12. The activatable antibody or antigen binding fragment thereof according to claim 1, wherein the cleavable linker(s) comprise(s) one or more cleavage sites recognized by one or more tumor specific proteases.

13. The activatable antibody or antigen binding fragment thereof according to claim 1, wherein at least one cleavable linker comprises a cleavage site recognized by matriptase or a cleavage site recognized by a metalloproteinase.

14. An Antibody-drug conjugate (ADC), comprising the activatable antibody or antigen binding fragment thereof according to claim 1 and a linker-drug.

15. A Pharmaceutical composition comprising an activatable antibody or antigen binding fragment thereof according to claim 1, and a pharmaceutically acceptable excipient.

16. (canceled)

17. A method for treating cancer, an autoimmine disease, or an infectious disease, which comprises administering to a subject in need of said treatment a therapeutically effective amount of the activatable antibody or antigen binding fragment thereof according to claim 1.

18. A nucleic acid construct comprising:
a nucleotide sequence encoding a heavy chain variable domain, a protease cleavable peptidic linker and a masking moiety; and/or
a nucleotide sequence encoding a light chain variable domain, a cleavable peptidic linker and a masking moiety;
wherein the nucleotide sequences are operably linked to an expression control sequence for expression in a host cell.

19. A host cell comprising a nucleic acid construct according to claim 18.

20. A method for producing an activatable antibody or antigen binding fragment thereof according to claim 1,
the method comprising the step of culturing a host cell comprising a nucleic acid construct;
wherein said nucleic acid construct comprises:
a nucleotide sequence encoding a heavy chain variable domain, a protease cleavable peptidic linker and a masking moiety; and/or
a nucleotide sequence encoding a light chain variable domain, a cleavable peptidic linker and a masking moiety;
wherein the nucleotide sequences are operably linked to an expression control sequence for expression in a host cell; and
wherein said culturing is under conditions conducive to expression of the activatable antibody or antigen binding fragment thereof.
